(19) United States
(12) Patent Application Publication
(10) Pub. No.: US 2022/0323414 A1
(43) Pub. Date: Oct. 13, 2022

(54) FARNESOID X RECEPTOR AGONISTS FOR THE TREATMENT OF DISEASE

(71) Applicant: Metacrine, Inc., San Diego, CA (US)

(72) Inventors: Kenneth SONG, San Diego, CA (US); Hubert CHEN, San Diego, CA (US); Brandee WAGNER, San Diego, CA (US)

(21) Appl. No.: 17/836,905

(22) Filed: Jun. 9, 2022

Related U.S. Application Data
(63) Continuation of application No. 17/276,766, filed on Mar. 16, 2021, now abandoned.
(60) Provisional application No. 62/733,008, filed on Sep. 18, 2018.

(51) Int. Cl. A61K 31/415; A61P 1/16; A61K 9/00; A61K 45/06

(52) U.S. Cl. CPC A61K 31/415; A61P 1/16; A61K 9/0053; A61K 45/06

(57) ABSTRACT

Described herein are combination therapies with farnesoid X receptor (FXR) agonists, and methods of using such pharmaceutical compositions in the treatment of conditions, diseases, or disorders associated with farnesoid X receptor activity.

### CROSS-REFERENCE

This application is a continuation of U.S. patent application Ser. No. 17/276,766, filed Mar. 16, 2021, which is a U.S. National Application of International Application No. PCT/US2019/051604, filed Sep. 17, 2019, which claims the benefit of U.S. Provisional Application No. 62/733,008, filed on Sep. 18, 2018, which is herein incorporated by reference in its entirety.

### FIELD OF THE INVENTION

Described herein are therapeutic strategies for the treatment of conditions, diseases, or disorders associated with farnesoid X receptor activity that include a farnesoid X receptor agonist, alone or in combination with other therapeutic agents.

### BACKGROUND OF THE INVENTION

Farnesoid X receptor (FXR) is a nuclear receptor expressed in the liver, intestine, kidney, and adipose tissue. FXR regulates a wide variety of target genes involved in the control of bile acid synthesis and transport, lipid metabolism, and glucose homeostasis. FXR agonism is a treatment modality for many metabolic and liver conditions.

### SUMMARY OF THE INVENTION

In one aspect, described herein is a method of treating or preventing a liver disease or condition, a lipid disease or disorder, a metabolic inflammation-mediated disease or disorder, or a combination thereof comprising administering to a subject in need thereof a compound that has the following structure of Compound 1:

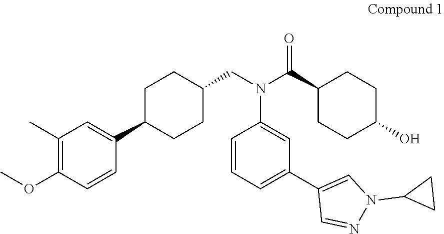 [SMILES: COc1ccc([C@H]2CC[C@H](CN(c3cccc(-c4cnn(C5CC5)c4)c3)C(=O)[C@H]3CC[C@H](O)CC3)CC2)cc1C]

or a pharmaceutically acceptable salt or solvate thereof.

In some embodiments, the liver disease or condition is steatohepatitis, cholangitis, fatty liver disease, cholestasis, cirrhosis, fibrotic liver disease, liver inflammation, primary biliary cholangitis, biliary atresia, Alagille syndrome, IFALD (intestinal failure associated liver disease), parental nutrition associated liver disease (PNALD), hepatitis, hepatocellular carcinoma, cholangiocarcinoma or combinations thereof.

In some embodiments, the steatohepatitis is nonalcoholic steatohepatitis (NASH), alcoholic steatohepatitis (ASH), or HIV-associated steatohepatitis.

In some embodiments, the liver disease or condition is nonalcoholic steatohepatitis (NASH).

In some embodiments, the liver disease or condition is NASH that is accompanied by liver fibrosis.

In some embodiments, the liver disease or condition is NASH without liver fibrosis.

In some embodiments, the cholangitis is primary biliary cholangitis (PBC) or primary sclerosing cholangitis (PSC).

In some embodiments, the fatty liver disease is non-alcoholic fatty liver disease (NAFLD) or alcohol-related fatty liver disease.

In some embodiments, the cholestasis is intrahepatic cholestasis or extrahepatic cholestasis.

In some embodiments, the cholestasis is intrahepatic cholestasis of pregnancy or progressive familial intrahepatic cholestasis (PFIC).

In some embodiments, the cirrhosis is HIV-associated cirrhosis.

In some embodiments, the metabolic inflammation-mediated disease or disorder is diabetes mellitus.

In some embodiments, the diabetes mellitus is diabetes mellitus type 2.

In some embodiments, the lipid disease or disorder is dyslipidemia.

In some embodiments, the fibrotic liver disease is a fibrotic liver disease resulting from nonalcoholic steatohepatitis (NASH), alcoholic steatohepatitis (ASH), non-alcoholic fatty liver disease (NAFLD), primary biliary cholangitis (PBC), primary sclerosing cholangitis (PSC), hepatitis C virus (HCV), cirrhosis, Wilson's disease, HIV associated steatohepatitis, HIV associated cirrhosis, or congenital hepatic fibrosis.

In some embodiments, the liver inflammation is acute hepatitis, chronic hepatitis, fulminant hepatitis, viral hepatitis, bacterial hepatitis, parasitic hepatitis, toxic- and drug-induced hepatitis, alcoholic hepatitis, autoimmune hepatitis, non-alcoholic steatohepatitis (NASH), neonatal hepatitis, or ischemic hepatitis.

In some embodiments, the hepatitis is autoimmune hepatitis.

In some embodiments, the liver disease or condition is Alagille syndrome.

In some embodiments, the liver disease or condition is biliary atresia.

In some embodiments, the liver disease or condition is hepatocellular carcinoma.

In some embodiments, the liver disease or condition is cholangiocarcinoma.

In some embodiments, Compound 1, or a pharmaceutically acceptable salt or solvate thereof, is systemically administered to the subject.

In some embodiments, Compound 1, or a pharmaceutically acceptable salt or solvate thereof, is administered to the subject orally, by injection, or intravenously.

In some embodiments, at least one additional therapeutic agent is administered to the subject in addition to Compound 1, or a pharmaceutically acceptable salt or solvate thereof.

In another aspect, described herein is a method of treating or preventing a gastrointestinal disease or condition, comprising administering to a subject in need thereof a compound that has the following structure of Compound 1:

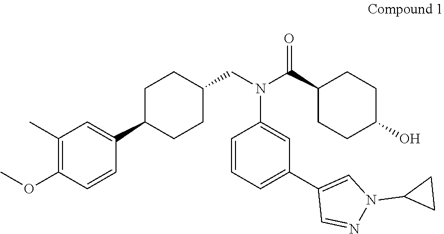 [SMILES: COc1ccc([C@H]2CC[C@H](CN(c3cccc(-c4cnn(C5CC5)c4)c3)C(=O)[C@H]3CC[C@H](O)CC3)CC2)cc1C]

or a pharmaceutically acceptable salt or solvate thereof.

In some embodiments, the gastrointestinal disease or condition is necrotizing enterocolitis, inflammatory bowel disease (IBD), irritable bowel syndrome (IBS), gastroenteritis, radiation induced enteritis, pseudomembranous colitis, enteritis, celiac disease, post-surgical inflammation of the intestines, graft versus host disease, bile acid reflux or colorectal cancer.

In some embodiments, the gastrointestinal disease or condition is inflammatory bowel disease (IBD).

In some embodiments, the inflammatory bowel disease (IBD) is Crohn's disease or ulcerative colitis.

In some embodiments, the irritable bowel syndrome (IBS) is irritable bowel syndrome with diarrhea (IBS-D), irritable bowel syndrome with constipation (IBS-C), mixed IBS (IBS-M), unsubtyped IBS (IBS-U), or bile acid diarrhea (BAD).

In some embodiments, the IBS-D is due to bile acid malabsorption.

In some embodiments, the gastrointestinal disease or condition is colitis. In some embodiments, the colitis is ulcerative colitis, microscopic colitis, or pseudomembranous colitis.

In some embodiments, the enteritis is radiation-induced enteritis or chemotherapy-induced enteritis.

In some embodiments, the gastroenteritis is idiopathic gastroenteritis.

In some embodiments, the gastrointestinal disease or condition is bile acid reflux that is accompanied by gastro-esophageal reflux disease (GERD).

In some embodiments, the gastrointestinal disease or condition is bile acid reflux without GERD.

In some embodiments, Compound 1, or a pharmaceutically acceptable salt or solvate thereof, is systemically administered to the subject. In some embodiments, Compound 1, or a pharmaceutically acceptable salt or solvate thereof, is non-systemically administered to the subject. In some embodiments, Compound 1, or a pharmaceutically acceptable salt or solvate thereof, is administered to the subject orally, by injection or intravenously.

In another aspect, described herein is a method of treating or preventing a renal disease or condition, comprising administering to a subject in need thereof a compound that has the following structure of Compound 1:

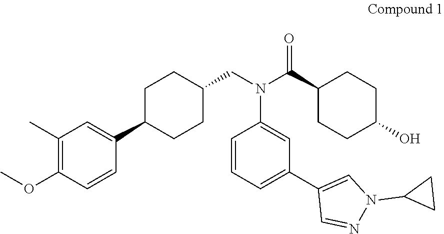 [SMILES: COc1ccc([C@H]2CC[C@H](CN(c3cccc(-c4cnn(C5CC5)c4)c3)C(=O)[C@H]3CC[C@H](O)CC3)CC2)cc1C]

or a pharmaceutically acceptable salt or solvate thereof

In some embodiments, the renal disease or condition is kidney fibrosis, acute kidney injury, chronic kidney injury, ischemic nephropathy, diabetic nephropathy, tubulointerstitial nephritis/nephropathy, glomerulonephritis/nephropathy, or combinations thereof.

In some embodiments, Compound 1, or a pharmaceutically acceptable salt or solvate thereof, is systemically administered to the subject.

In some embodiments, Compound 1, or a pharmaceutically acceptable salt or solvate thereof, is administered to the subject orally, by injection or intravenously.

In another aspect, described herein is a method of treating or preventing cancer, comprising administering to a subject in need thereof a compound that has the following structure of Compound 1:

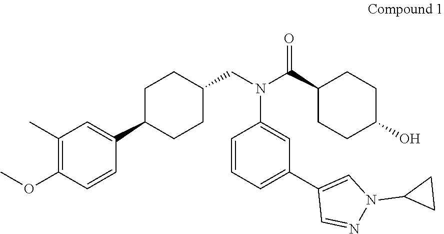 [SMILES: COc1ccc([C@H]2CC[C@H](CN(c3cccc(-c4cnn(C5CC5)c4)c3)C(=O)[C@H]3CC[C@H](O)CC3)CC2)cc1C]

or a pharmaceutically acceptable salt or solvate thereof.

In some embodiments, the cancer is prostate cancer, colorectal cancer, or hepatocellular carcinoma.

In some embodiments, Compound 1, or a pharmaceutically acceptable salt or solvate thereof, is systemically administered to the subject.

In some embodiments, Compound 1, or a pharmaceutically acceptable salt or solvate thereof, is administered to the subject orally, by injection or intravenously.

Articles of manufacture, which include packaging material, a compound described herein, or a pharmaceutically acceptable salt thereof, within the packaging material, and a label that indicates that a FXR agonist (e.g. Compound 1, or a pharmaceutically acceptable salt thereof), is used for modulating the activity of FXR, or for the treatment, prevention or amelioration of one or more symptoms of a disease or condition that would benefit from modulation of FXR activity, are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE FIGURES

FIG. 1 shows Compound 1 drug levels in plasma following 14 days of once daily oral dosing

FIG. 2 shows the percent reduction of total bile acids in healthy patients dosed with the indicated amount of Compound 1.

FIG. 3A shows the change in C4 levels in healthy patients receiving the indicated dose of Compound 1 for a period of 24 hours following administration.

FIG. 3B shows the change in FGF19 levels in healthy patients receiving the indicated dose of Compound 1 for a period of 24 hours following administration.

FIG. 4 shows the change in relative hepatic fat in NASH patients receiving daily 50 mg doses of Compound 1 over a 28 day period.

FIG. 5A shows the percent change from baseline of LDL-C levels in NASH patients receiving daily 50 mg doses of Compound 1 over a 28 day period.

FIG. 5B shows the percent change from baseline of triglyceride levels in NASH patients receiving daily 50 mg doses of Compound 1 over a 28 day period.

FIG. 5C shows the percent change from baseline of HDL-C levels in NASH patients receiving daily 50 mg doses of Compound 1 over a 28 day period.

FIG. 6A shows the percent change from baseline of ALT levels in NASH patients receiving daily 50 mg doses of Compound 1 over a 28 day period.

FIG. 6B shows the percent change from baseline of GGT levels in NASH patients receiving daily 50 mg doses of Compound 1 over a 28 day period.

FIG. 7 shows pharmacokinetic results in healthy and NASH patients dosed with Compound 1 at 50 mg.

### DETAILED DESCRIPTION OF THE INVENTION

FXR plays a pivotal role in suppressing inflammation in the liver and regulating lipid metabolism. The nuclear hormone receptor farnesoid X receptor (also known as FXR or nuclear receptor subfamily 1, group H, member 4 (NR1H4)) (OMIM: 603826) functions as a regulator for bile acid metabolism. FXR is a ligand-activated transcriptional receptor expressed in diverse tissues including the adrenal gland, kidney, stomach, duodenum, jejunum, ileum, colon, gall bladder, liver, macrophages, and white and brown adipose tissue. Bile acids function as endogenous ligands for FXR such that enteric and systemic release of bile acids induces FXR-directed changes in gene expression networks. Bile acids are the primary oxidation product of cholesterol, and in some cases, upon secretion into the intestines, are regulators of cholesterol absorption. The rate-limiting step for conversion of cholesterol into bile acids is catalyzed by cytochrome p450 enzyme cholesterol 7-α-hydroxylase (CYP7A1) and occurs in the liver. Activation of FXR represses the transcription of CYP7A1 by increasing the expression level of the hepatic small heterodimer partner (SHP) (also known as nuclear receptor subfamily 0, group B, member 2; or NR0B2) and intestinal expression of fibroblast growth factor 15 (FGF15) in mice and fibroblast growth factor 19 (FGF19) in human. SHP represses the liver receptor homolog (LRH-1), a nuclear receptor necessary for CYP7A1 gene expression, through its interaction with LRH-1 to form a non-functional heterodimer. In some cases, FGF15/19 released from the intestine then activates the fibroblast growth factor receptor 4 in the liver, leading to activation of the mitogen-activated protein kinase (MAPK) signaling pathway which suppresses Cyp7A1.

In some embodiments, the activation of FXR leads to a reduction in hepatic inflammation. For example, it has been shown the activation of FXR antagonizes the NF-κB pathway involved in hepatic inflammation (Wang et al., Hepatology 48(5): 1632-1643, 2008). In some embodiments, the activation of FXR reduces gastrointestinal tract inflammation. For example, activation of FXR decreases the production of inflammatory cytokines such as interleukin (IL) 1-beta, IL-2, and IL-6, tumor necrosis factor-alpha (TNF-α), and interferon-gamma (Stojancevic et al., Can J Gastroenterol, 26(9): 631-637, 2012).

There is an unmet need for therapeutics that specifically focus on the molecular targets and/or pathways involved in liver diseases such as fibrotic, metabolic, and inflammatory liver diseases.

Disclosed herein, in certain embodiments, are methods of treating a liver disease in a subject in need thereof, comprising administering to the subject a farnesoid X receptor (FXR) agonist.

Further disclosed herein, in certain embodiments, are methods of treating a metabolic liver disease in a subject in need thereof, comprising administering to the subject an FXR agonist.

Further disclosed herein, in certain embodiments, are methods of treating a fibrotic liver disease in a subject in need thereof, comprising administering to the subject an FXR agonist.

Further disclosed herein, in certain embodiments, are methods of treating a gastrointestinal disease in a subject in need thereof, comprising administering to the subject a farnesoid X receptor (FXR) agonist.

Further disclosed herein, in certain embodiments, are methods of treating inflammation in a subject in need thereof, comprising administering to the subject a farnesoid X receptor (FXR) agonist.

Additionally disclosed herein, in certain embodiments, are pharmaceutical compositions comprising a farnesoid X receptor (FXR) agonist.

### Liver Diseases

Disclosed herein, in certain embodiments, are methods of treating or preventing a liver disease in a subject in need thereof, comprising administering to the subject a farnesoid X receptor (FXR) agonist. In some embodiments, at least one additional therapeutic agent is administered to the subject in addition to the FXR agonist. In some embodiments, the FXR agonist is Compound 1, or a pharmaceutically acceptable salt thereof.

In some embodiments, the liver disease is alcoholic or non-alcoholic. In some embodiments, the liver disease is an alcoholic liver disease. Exemplary alcoholic liver diseases or conditions include, but are not limited to fatty liver (steatosis), cirrhosis, alcoholic steatohepatitis (ASH), or alcoholic hepatitis. In some embodiments, a farnesoid X receptor (FXR) agonist is administered to a subject in need thereof as a method of treating or preventing fatty liver (steatosis), cirrhosis, alcoholic steatohepatitis (ASH), or alcoholic hepatitis.

### Steatosis

Steatosis, also known as fatty change, adipose degeneration, or fatty degeneration, is the process describing abnormal retention of lipids within a cell. In some embodiments, an FXR agonist reduces steatosis in a mammal. In some examples, the FXR agonist reduces steatosis in the mammal by at least 5%, at least 10%, at least 15%, at least 20%, at least 30%, at least 40%, at least 50%, or more. In some instances, steatosis is reduced by about 5% to about 50%, by about 5% to about 25%, by about 10% to about 20%, or by about 10% to about 30%. In some instances, the level of steatosis is relative to the level of steatosis in a mammal not treated with the FXR agonist. In some embodiments, an additional therapeutic agent is administered to the mammal. In some embodiments, the additional therapeutic agent is an anti-inflammatory agent, metabolic agent or anti-fibrotic agent.

Hepatic steatosis, also known as fatty liver, is a condition wherein excessive amounts of triglyceride lipids accumulate in liver cells and can also accompanied by progressive inflammation of the liver which is also known as steatohepatitis. In some embodiments, an FXR agonist disclosed herein reduces fatty liver (hepatic steatosis) or steatohepatitis in a mammal. In some examples, the FXR agonist reduces hepatic steatosis or steatohepatitis in the mammal by at least 5%, at least 10%, at least 15%, at least 20%, at least 30%, at least 40%, at least 50%, or more. In some instances, hepatic steatosis or steatohepatitis is reduced by about 5% to about 50%, by about 5% to about 25%, by about 10% to about 20%, or by about 10% to about 30%. In some instances, the level of hepatic steatosis or steatohepatitis is relative to the level of hepatic steatosis or steatohepatitis in a mammal not treated with the FXR agonist. In some embodiments, an additional therapeutic agent is administered to the mammal. In some embodiments, the additional therapeutic agent is an anti-inflammatory agent, metabolic agent or anti-fibrotic agent.

### Cirrhosis

Cirrhosis is a condition in which the liver undergoes long term damage that affects its functions. Symptoms of cirrhosis include, but are not limited to fatigue, swelling in the lower legs, jaundice, easy bruising, fluid build-up in the abdomen, or spider-like blood vessels. Cirrhosis is most commonly caused by alcohol, hepatitis B, hepatitis C, and non-alcoholic liver disease. In some embodiments, an FXR agonist disclosed herein reduce cirrhosis in a mammal. In some examples, an FXR agonist reduces cirrhosis in the mammal by at least 5%, at least 10%, at least 15%, at least 20%, at least 30%, at least 40%, at least 50%, or more. In some instances, the level of cirrhosis is reduced by about 5% to about 50%, by about 5% to about 25%, by about 10% to about 20%, or by about 10% to about 30%. In some instances, the level of cirrhosis is relative to the level of cirrhosis in a mammal not treated with the FXR agonist. In some embodiments, an additional therapeutic agent is administered to the mammal. In some embodiments, the additional therapeutic agent is an anti-inflammatory agent, metabolic agent or anti-fibrotic agent.

### Alcoholic Steatohepatitis (ASH)

Alcoholic steatohepatitis is a condition wherein excessive amounts of triglyceride lipids accumulate in liver cells due to chronic intake of alcohol and can be accompanied by progressive inflammation of the liver. In some embodiments, an FXR agonist disclosed herein reduce alcoholic steatohepatitis in a mammal. In some examples, an FXR agonist reduces alcoholic steatohepatitis in the mammal by at least 5%, at least 10%, at least 15%, at least 20%, at least 30%, at least 40%, at least 50%, or more. In some instances, the level of alcoholic steatohepatitis is reduced by about 5% to about 50%, by about 5% to about 25%, by about 10% to about 20%, or by about 10% to about 30%. In some instances, the level of alcoholic steatohepatitis is relative to the level of alcoholic steatohepatitis in a mammal not treated with the FXR agonist. In some embodiments, an additional therapeutic agent is administered to the mammal. In some embodiments, the additional therapeutic agent is an anti-inflammatory agent, metabolic agent or anti-fibrotic agent.

### Alcoholic Hepatitis

Alcoholic hepatitis is inflammation in the liver due to the excessive intake of alcohol. It is usually associated with fatty liver and contributes to the progression of fibrosis, which leads to cirrhosis. In some embodiments, an FXR agonist disclosed herein reduce alcoholic hepatitis in a mammal. In some examples, an FXR agonist reduces alcoholic hepatitis in the mammal by at least 5%, at least 10%, at least 15%, at least 20%, at least 30%, at least 40%, at least 50%, or more. In some instances, the level of alcoholic hepatitis is reduced by about 5% to about 50%, by about 5% to about 25%, by about 10% to about 20%, or by about 10% to about 30%. In some instances, the level of alcoholic hepatitis is relative to the level of alcoholic hepatitis in a mammal not treated with the FXR agonist. In some embodiments, an additional therapeutic agent is administered to the mammal. In some embodiments, the additional therapeutic agent is an anti-inflammatory agent, metabolic agent or anti-fibrotic agent.

### Metabolic Liver Diseases

In some embodiments, a farnesoid X receptor (FXR) agonist is administered to a subject in need thereof as a method of treating or preventing a non-alcoholic liver disease. In some embodiments, the non-alcoholic liver disease is a metabolic liver disease. In some embodiments, the metabolic disease is accompanied by liver fibrosis. In some embodiments, the metabolic liver disease is caused by obesity, hypertension, dyslipidemia, type 2 diabetes, impaired glucose tolerance, impaired fasting glycaemia, or insulin resistance.

Disclosed herein, in certain embodiments, are methods of treating or preventing a metabolic liver disease in a subject in need thereof, comprising administering to the subject a farnesoid X receptor (FXR) agonist. In some embodiments, the metabolic liver disease is nonalcoholic fatty liver disease (NAFLD), intrahepatic cholestasis, or extrahepatic cholestasis. In some embodiments, a farnesoid X receptor (FXR) agonist is administered to a subject in need thereof as a method of treating or preventing nonalcoholic fatty liver disease (NAFLD), intrahepatic cholestasis, or extrahepatic cholestasis.

In some embodiments, regulation of metabolic processes such as bile acid synthesis, bile-acid circulation, glucose metabolism, lipid metabolism, or insulin sensitivity is modulated by the activation of FXR. Furthermore, in some embodiments, dis-regulation of metabolic processes such as bile acid synthesis, bile-acid circulation, glucose metabolism, lipid metabolism, or insulin sensitivity results in metabolic diseases such as diabetes or diabetes-related conditions or disorders, alcoholic or non-alcoholic liver diseases or conditions, intestinal inflammation, or cell proliferative disorders.

In some embodiments, elevated levels of bile acids have been associated with insulin resistance. For example, insulin resistance sometimes leads to a decreased uptake of glucose from the blood, and increased de novo glucose production in the liver. In some instances, intestinal sequestration of bile acids has been shown to improve insulin resistance by promoting the secretion of glucagon-like peptide-1 (GLP-1) from intestinal L-cells. GLP-1 is an incretin derived from the transcription product of the proglucagon gene. It is released in response to the intake of food and exerts control in appetite and gastrointestinal function, and promotes insulin secretion from the pancreas. The biologically active forms of GLP-1 include GLP-1-(7-37) and GLP-1-(7-36)NH₂, which result from selective cleavage of the proglucagon molecule.

In some embodiments, the activation of FXR also correlates to the secretion of pancreatic polypeptide-fold such as peptide YY (PYY or PYY3-36). In some instances, peptide YY is a gut hormone peptide that modulates neuronal activity within the hypothalamic and brainstem, regions of the brain involved in reward processing. In some instances, reduced level of PYY correlates to increased appetite and weight gain.

In some instances, the activation of FXR indirectly leads to a reduction of plasma triglycerides. The clearance of triglycerides from the bloodstream is due to lipoprotein lipase (LPL). LPL activity is enhanced by the induction of its activator apolipoprotein CII, and the repression of its inhibitor apolipoprotein CIII in the liver occurs upon FXR activation.

In some cases, the activation of FXR further modulates energy expenditure such as adipocyte differentiation and function. Adipose tissue comprises adipocytes or fat cells. In some instances, adipocytes are further differentiated into brown adipose tissue (BAT) or white adipose tissue (WAT). The function of BAT is to generate body heat, while WAT functions as fat storing tissues. In some embodiments, the activation of FXR enhances thermogenesis and browning of WAT. In some embodiments, the activation of FXR increases BAT mass.

In some instances, FXR is widely expressed in the intestine. In some cases, the activation of FXR has been shown to induce the expression and secretion of FGF19 (or FGF15 in mouse) in the intestine. FGF19 is a hormone that regulates bile acid synthesis as well as exerts an effect on glucose metabolism, lipid metabolism, and on energy expenditure. In some instances, FGF19 has also been observed to modulate adipocyte function and differentiation. Indeed, a study has shown that the administration of FGF19 to high-fat diet-fed mice increased energy expenditure, modulated adipocytes differentiation and function, reversed weight gain, and improved insulin resistance (see, Fu et al., “Fibroblast growth factor 19 increases metabolic rate and reverses dietary and leptin-deficient diabetes.” Endocrinology 145:2594-2603 (2004)).

In some cases, intestinal FXR activity has also been shown to be involved in reducing overgrowth of the microbiome, such as during feeding (Li et al., Nat Commun 4:2384, 2013). For example, a study showed that activation of FXR correlated with increased expression of several genes in the ileum such as Ang2, iNos, and Il18, which have established antimicrobial actions (Inagaki et al., Proc Natl Acad Sci USA 103:3920-3925, 2006).

G protein-coupled bile acid receptor 1 (also known as GPBAR2, GPCR19, membrane-type receptor for bile acids or M-BAR, or TGR5) is a cell surface receptor for bile acids. Upon activation with bile acid, TGR5 induces the production of intracellular cAMP, which then triggers an increase in triiodothyronine due to the activation of deiodinase (D1O2) in BAT, resulting in increased energy expenditure.

### Non-Alcoholic Fatty Liver Disease (NAFLD)

Non-alcoholic fatty liver disease (NAFLD) is associated with excessive fat in the liver (steatosis) due to causes other than excessive alcohol intake. NAFLD can manifest as simple steatosis or steatosis with inflammation and liver injury which is classified as non-alcoholic steatohepatitis (NASH). NAFLD is related to metabolic syndrome and insulin resistance. Metabolic syndrome is a clustering of at least three medical conditions, which include, but are not limited to obesity, elevated blood pressure, elevated fasting plasma glucose, high serum triglycerides, or high low-density lipoprotein (LDL) levels.

In some embodiments, an FXR agonist disclosed herein is used in the treatment of NAFLD. In some examples, an FXR agonist reduces NAFLD in the mammal by at least 5%, at least 10%, at least 15%, at least 20%, at least 30%, at least 40%, at least 50%, or more. In some cases, NAFLD is reduced by about 5% to about 50%, by about 5% to about 25%, by about 10% to about 20%, or by about 10% to about 30%. In some instances, the level of NAFLD is relative to the level of NAFLD in a mammal not treated with the FXR agonist. In some embodiments, an additional therapeutic agent is administered to the mammal. In some embodiments, the additional therapeutic agent is an anti-inflammatory agent, metabolic agent or anti-fibrotic agent.

### Cholestasis

Cholestasis is an impairment or cessation in the flow of bile, which in some cases, causes hepatotoxicity due to the buildup of bile acids and other toxins in the liver. In some embodiments, cholestasis is intrahepatic cholestasis or extrahepatic cholestasis. In some embodiments, intrahepatic cholestasis is caused by amyloidosis, bacterial abscess in the liver, being fed exclusively intravenously, lymphoma, pregnancy, primary biliary cholangitis, primary or metastatic liver cancer, cholangiocarcinoma, primary sclerosing cholangitis, sarcoidosis, serious infections that have spread through the bloodstream (sepsis), tuberculosis, or viral hepatitis. In some embodiments, extrahepatic cholestasis is caused by bile duct tumors, cysts narrowing of the bile duct (strictures), stones in the common bile duct, pancreatitis, pancreatic tumor or pseudocyst, pressure on the bile ducts due to a nearby mass or tumor, or primary sclerosing cholangitis. In some embodiments, cholestasis is caused by a drug. In some embodiments, cholestasis is caused by antibiotics such as ampicillin and other penicillins, anabolic steroids, oral contraceptive pills, chlorpromazine, cimetidine, estradiol, imipramine, prochlorperazine, terbinafine, or tolbutamide.

In some instances, cholestasis is a component of many liver diseases, including but not limited to cholelithiasis, cholestasis of pregnancy, primary biliary cholangitis (PBC), and primary sclerosing cholangitis (PSC). In some instances, the obstruction is due to gallstones, biliary trauma, drugs, one or more additional liver diseases, or to cancer. In some cases, the enterohepatic circulation of bile acids enables the absorption of fats and fat-soluble vitamins from the intestine and allows the elimination of cholesterol, toxins, and metabolic by-products such as bilirubin from the liver. In some cases, activation of FXR induces expression of the canalicular bile transporters BSEP (ABCB11) and multidrug resistance—related protein 2 (MRP2; ABCC2, cMOAT), and represses genes involved in bile acid biosynthesis, such as for example sterol 12α-hydroxylase (CYP8B1) and CYP7A1.

In some embodiments, an FXR agonist disclosed herein is used in the treatment of cholestasis in a mammal. In some examples, an FXR agonist reduces cholestasis in the mammal by at least 5%, at least 10%, at least 15%, at least 20%, at least 30%, at least 40%, at least 50%, or more. In some cases, cholestasis is reduced by about 5% to about 50%, by about 5% to about 25%, by about 10% to about 20%, or by about 10% to about 30%. In some instances, the level of cholestasis is relative to the level of cholestasis in a mammal not treated with the FXR agonist. In some embodiments, an additional therapeutic agent is administered to the mammal.

In some embodiments, the additional therapeutic agent is an anti-inflammatory agent, metabolic agent or anti-fibrotic agent.

### Fibrotic Liver Diseases

Disclosed herein, in certain embodiments, are methods of treating or preventing a fibrotic liver disease in a subject in need thereof, comprising administering to the subject a farnesoid X receptor (FXR) agonist. In some embodiments, the fibrotic liver disease comprises liver fibrosis. In some embodiments, the fibrotic liver disease is caused by an alpha-1 antitrypsin deficiency, a copper storage disease, fructosemia, galactosemia, a glycogen storage disease, an iron-overload syndrome, a lipid abnormality, a peroxisomal disorder, tyrosinemia, a bacterial infection, a parasitic infection, a viral infection, by a disorder affecting hepatic blood flow, a drug or a chemical, or a mechanical obstruction. In some embodiments, the fibrotic liver disease disorder affecting hepatic blood flow is Budd-Chiari syndrome, heart failure, hepatic veno-occlusive disease, or portal vein thrombosis. In some embodiments, the drug or chemical causing the fibrotic liver disease is amiodarone, chlorpromazine, isoniazid, methotrexate, methyldopa, oxyphenisatin, alcohol, or tolbutamide. In some embodiments, the mechanical obstruction causing a fibrotic liver disease is hepatic scarring due to liver surgery or bile duct strictures due to impacted gallstones.

In some embodiments, the fibrotic liver disease is nonalcoholic steatohepatitis (NASH), alcoholic hepatitis, primary biliary cholangitis, primary sclerosing cholangitis, congenital hepatic fibrosis, or autoimmune hepatitis.

### Liver Fibrosis

Liver fibrosis is not an independent disease, but rather a histological change in the liver comprising abnormal amounts of collagen fiber deposits in the extracellular spaces of the liver cells. Liver fibrosis is caused by liver inflammation and liver damage. Liver damage causes activated hepatic stellate cells to increase production and accumulation of extracellular matrix (ECM) proteins, leading to hardening of the liver cells and increased loss of blood infusion into the liver.

In some embodiments, an FXR agonist disclosed herein is used in the treatment of liver fibrosis in a mammal. In some examples, an FXR agonist reduces liver fibrosis in the mammal by at least 5%, at least 10%, at least 15%, at least 20%, at least 30%, at least 40%, at least 50%, or more. In some cases, liver fibrosis is reduced by about 5% to about 50%, by about 5% to about 25%, by about 10% to about 20%, or by about 10% to about 30%. In some instances, the level of liver fibrosis is relative to the level of liver fibrosis in a mammal not treated with the FXR agonist. In some embodiments, an additional therapeutic agent is administered to the mammal. In some embodiments, the additional therapeutic agent is an anti-inflammatory agent, metabolic agent, or anti-fibrotic agent.

### Non-Alcoholic Steatohepatitis (NASH)

Non-alcoholic fatty liver disease (NAFLD) is associated with excessive fat in the liver (steatosis) and in some cases progresses to NASH, which is defined by the histologic hallmarks of inflammation, cell death, and fibrosis. In some instances, primary NASH is associated with insulin resistance, while secondary NASH is caused by medical or surgical conditions or drugs such as, but not limited to, tamoxifen. In some cases, NASH progresses to advanced fibrosis, hepatocellular carcinoma, or end-stage liver disease requiring liver transplantation.

In some instances, NASH develops as a result of triglyceride (TGs) imbalance. For example, dysfunctional adipocytes secrete pro-inflammatory molecules such as cytokines and chemokines leading to insulin resistance and a failure of lipolysis suppression in the adipocytes. In some instances, this failure of lipolysis suppression leads to a release of free fatty acids (FFAs) into the circulation and uptake within the liver. In some cases, over-accumulation of FFAs in the form of triglycerides (TGs) in lipid droplets leads to oxidative stress, mitochondrial dysfunction, and upregulation of pro-inflammatory molecules.

In some instances, activation of FXR inhibits triglyceride (TG)/fatty acid (FA) synthesis facilitated by suppressing sterol regulatory element-binding protein 1c (SREBP1c) via activation of SHP. In some cases, FXR additionally increases the clearance of TG by stimulating lipoprotein lipase (LPL) activity as well as the hepatic uptake of remnants and low-density lipoprotein by inducing syndecan 1 (SDC1) and the VLDL receptor (VLDLR).

In some embodiments, an FXR agonist disclosed herein is used in the treatment of non-alcoholic steatohepatitis (NASH). In some examples, an FXR agonist reduce NASH scores by at least 5%, at least 10%, at least 15%, at least 20%, at least 30%, at least 40%, at least 50%, or more. In some cases, NASH is reduced by about 5% to about 50%, by about 5% to about 25%, by about 10% to about 20%, or by about 10% to about 30%. In some instances, the level of NASH is relative to the level of NASH in a mammal not treated with an FXR agonist. In some embodiments, an additional therapeutic agent is administered to the mammal. In some embodiments, the additional therapeutic agent is an anti-inflammatory agent, metabolic agent, or anti-fibrotic agent.

### Primary Biliary Cholangitis (PBC)

PBC is a liver disease that primarily results from an autoimmune destruction of the bile ducts that transport bile acids (BAs) out of the liver, resulting in cholestasis. As PBC progresses, persistent toxic buildup of BAs causes progressive liver damage. Chronic inflammation and fibrosis advance to cirrhosis. PBC is a chronic, progressive disorder whose symptoms typically develop in middle age. Current treatments for PBC include ursodeoxycholic acid (UDCA). Other FXR agonists have also been explored as potential therapies as well. Increased FXR activity is linked with reduced bile acid synthesis which can alleviate the buildup of bile acids in the liver associated with PBC. In some embodiments, an FXR agonist disclosed herein is used in the treatment of primary biliary cholangitis (PBC) in a mammal. In some examples, an FXR agonist reduces PBC in the mammal by at least 5%, at least 10%, at least 15%, at least 20%, at least 30%, at least 40%, at least 50%, or more. In some cases, PBC is reduced by about 5% to about 50%, by about 5% to about 25%, by about 10% to about 20%, or by about 10% to about 30%. In some instances, the level of PBC is relative to the level of PBC in a mammal not treated with the FXR agonist. In some embodiments, an additional therapeutic agent is administered to the mammal. In some embodiments, the additional therapeutic agent is an anti-inflammatory agent, metabolic agent, or anti-fibrotic agent.

### Primary Sclerosing Cholangitis (PSC)

PSC is a chronic and progressive cholestatic liver disease. PSC is characterized by progressive inflammation, fibrosis, and stricture formation in liver ducts. Common symptoms include pruritus and jaundice. The disease is strongly associated with inflammatory bowel disease (IBD); about 5% of patients with ulcerative colitis will have PSC. Up to 70% of patients with PSC also have IBD, most commonly ulcerative colitis. In some embodiments, an FXR agonist disclosed herein is used in the treatment of primary sclerosing cholangitis (PSC). In some examples, an FXR agonist reduces PSC in the mammal by at least 5%, at least 10%, at least 15%, at least 20%, at least 30%, at least 40%, at least 50%, or more. In some cases, PSC is reduced by about 5% to about 50%, by about 5% to about 25%, by about 10% to about 20%, or by about 10% to about 30%. In some instances, the level of PSC is relative to the level of PSC in a mammal not treated with the FXR agonist. In some embodiments, an additional therapeutic agent is administered to the mammal. In some embodiments, the additional therapeutic agent is an anti-inflammatory agent, metabolic agent, or anti-fibrotic agent.

### Congenital or Neonatal Liver Diseases

Congenital or neonatal liver diseases include, but are not limited to, congenital hepatic fibrosis, biliary atresia, Alagille syndrome, Progressive familial intrahepatic cholestasis-1 (PFIC-1), PFIC-2, PFIC-3, alpha-1 antitrypsin deficiency, choledochal cyst, and Wilson's disease. In some embodiments, congenital or neonatal liver diseases are orphan liver diseases.

Congenital hepatic fibrosis is a rare, inherited disease that is associated with an abnormal development of the portal veins and bile ducts and periportal fibrosis that leads to portal hypertension. In some embodiments, an FXR agonist disclosed herein is used in the treatment of congenital hepatic fibrosis in a mammal. In some examples, an FXR agonist reduces congenital hepatic fibrosis in the mammal by at least 5%, at least 10%, at least 15%, at least 20%, at least 30%, at least 40%, at least 50%, or more. In some cases, congenital hepatic fibrosis is reduced by about 5% to about 50%, by about 5% to about 25%, by about 10% to about 20%, or by about 10% to about 30%. In some instances, the level of congenital hepatic fibrosis is relative to the level of congenital hepatic fibrosis in a mammal not treated with the FXR agonist. In some embodiments, an additional therapeutic agent is administered to the mammal. In some embodiments, the additional therapeutic agent is an anti-inflammatory agent, metabolic agent, or anti-fibrotic agent.

Biliary atresia, also known as extrahepatic ductopenia or progressive obliterative cholangiopathy, is a rare medical condition that occurs in infants where bile ducts develop abnormally before birth and consequently become inflamed and/or obstructed after birth. This obstruction leads to a buildup of bile acids and other compounds that can cause damage to the liver. The condition affects about one in 15,000 babies. Symptoms of biliary atresia include jaundice, dark urine, alcoholic stool, weight loss, and irritability. Children with the disease cannot properly digest fats and may suffer from a loss of vitamins or protein. Left untreated, the condition can lead to death. There are currently no medications for the treatment of biliary atresia, with surgery being required for treatment. Individuals with biliary atresia display elevated bile acid levels in the blood and plasma. Additionally, patients with biliary atresia also show reduced expression of FXR. Increased FXR activity is linked with reduced bile acid synthesis which can alleviate the buildup of bile acids in the liver associated with biliary atresia. In some embodiments, an FXR agonist disclosed herein is used in the treatment of biliary atresia in a mammal. In some examples, an FXR agonist reduces biliary atresia in the mammal by at least 5%, at least 10%, at least 15%, at least 20%, at least 30%, at least 40%, at least 50%, or more. In some cases, biliary atresia is reduced by about 5% to about 50%, by about 5% to about 25%, by about 10% to about 20%, or by about 10% to about 30%. In some instances, the level of biliary atresia is relative to the level of biliary atresia in a mammal not treated with the FXR agonist. In some embodiments, an additional therapeutic agent is administered to the mammal. In some embodiments, the additional therapeutic agent is an anti-inflammatory agent, metabolic agent, or anti-fibrotic agent.

Alagille syndrome is an autosomal dominant genetic disorder that leads to biliary hypoplasia, biliary paucity, or biliary atresia, among other resulting conditions. In Alagille syndrome, bile duct abnormalities result in reduced ability to transport bile acids out of the liver. This results in the buildup of bile acids in the liver, which can cause scarring that prevents the liver from working properly. Treatments of the symptoms of Alagille syndrome include administration of ursodeoxycholic acid, an FXR agonist shown to help the flow of bile out of the liver. In some embodiments, an FXR agonist disclosed herein is used in the treatment of Allagille syndrome in a mammal. In some examples, an FXR agonist reduces Alagille syndrome in the mammal by at least 5%, at least 10%, at least 15%, at least 20%, at least 30%, at least 40%, at least 50%, or more. In some cases, Alagille syndrome is reduced by about 5% to about 50%, by about 5% to about 25%, by about 10% to about 20%, or by about 10% to about 30%. In some instances, the level of Alagille syndrome is relative to the level of Allagille syndrome in a mammal not treated with the FXR agonist. In some embodiments, an additional therapeutic agent is administered to the mammal. In some embodiments, the additional therapeutic agent is an anti-inflammatory agent, metabolic agent, or anti-fibrotic agent.

Progressive familial intrahepatic cholestasis (PFIC) is a group of inherited conditions that causes progressive cholestasis in infants and young adults, which leads to cirrhosis and eventually a need for liver transplantation. There are three variations of PFIC: PFIC-1, PFIC-2, and PFIC-3. PFIC-1 is caused by mutations in ATP8B1, a gene that codes for FIC-1, which is responsible for phospholipid translocation across membranes. PFIC-2 is caused by mutations in ABCB11, a gene that encodes for the bile salt export pump (BSEP). PFIC-3 is caused by mutations in ABCB4, a gene that encodes for multidrug resistance protein 3 (MDR3), which is responsible for phosphatidylcholine translocation. Since PFIC is associated with a buildup of bile acids in the liver, FXR agonists have been explored as potential therapies for PFIC. Some success has been seen in animal models, but patients receiving the treatment have seen dyslipidemia frequency increase in response. In some embodiments, an FXR agonist disclosed herein is used in the treatment of PFIC or any of its variations in a mammal. In some examples, an FXR agonist reduces PFIC or any of its variations in the mammal by at least 5%, at least 10%, at least 15%, at least 20%, at least 30%, at least 40%, at least 50%, or more. In some cases, PFIC or any of its variations is reduced by about 5% to about 50%, by about 5% to about 25%, by about 10% to about 20%, or by about 10% to about 30%. In some instances, the level of PFIC or any of its variations is relative to the level of PFIC in a mammal not treated with the FXR agonist. In some embodiments, an additional therapeutic agent is administered to the mammal. In some embodiments, the additional therapeutic agent is an anti-inflammatory agent, metabolic agent, or anti-fibrotic agent.

Alpha-1 antitrypsin deficiency is an inherited condition causing a defective production of alpha-1 antitrypsin (A1AT) leading to accumulation of A1AT in the liver. A1AT deficiency leads to a number of diseases including, but not limited to cirrhosis, autoimmune hepatitis, chronic obstructive pulmonary disorder (COPD), asthma, or emphysema. In some embodiments, an FXR agonist disclosed herein is used in the treatment of A1AT deficiency in a mammal. In some examples, an FXR agonist reduces an A1AT deficiency in the mammal by at least 5%, at least 10%, at least 15%, at least 20%, at least 30%, at least 40%, at least 50%, or more. In some cases, A1AT deficiency is reduced by about 5% to about 50%, by about 5% to about 25%, by about 10% to about 20%, or by about 10% to about 30%. In some instances, the level of A1AT deficiency is relative to the level of A1AT deficiency in a mammal not treated with the FXR agonist. In some embodiments, an additional therapeutic agent is administered to the mammal. In some embodiments, the additional therapeutic agent is an anti-inflammatory agent, metabolic agent, or anti-fibrotic agent.

A choledochal cyst is a congenital condition involving cystic dilation of bile ducts that further develops into cholangitis. Choledochal cysts are classified into: type I, type II, type III or choledochocele, type IVa, type IVb, type V, and type VI. In some embodiments, an FXR agonist disclosed herein is used in the treatment of choledochal cysts in a mammal. In some examples, an FXR agonist reduces a choledochal cyst in the mammal by at least 5%, at least 10%, at least 15%, at least 20%, at least 30%, at least 40%, at least 50%, or more. In some cases, a choledochal cyst is reduced by about 5% to about 50%, by about 5% to about 25%, by about 10% to about 20%, or by about 10% to about 30%. In some embodiments, the additional therapeutic agent is an anti-inflammatory agent, metabolic agent, or anti-fibrotic agent.

Wilson's disease is an autosomal recessive condition in which copper is not excreted properly from the body. Symptoms of Wilson's disease typically affect the brain and liver. Complications of Wilson's disease include, but are not limited to hepatic encephalopathy, portal hypertension, chronic active hepatitis, acute liver failure, hemolytic anemia, and splenomegaly. In some embodiments, an FXR agonist disclosed herein is used in the treatment of Wilson's disease or a complication of Wilson's disease in a mammal. In some examples, an FXR agonist and an additional therapeutic agent reduce Wilson's disease or a complication of Wilson's disease in the mammal by at least 5%, at least 10%, at least 15%, at least 20%, at least 30%, at least 40%, at least 50%, or more. In some cases, Wilson's disease or a complication of Wilson's disease is reduced by about 5% to about 50%, by about 5% to about 25%, by about 10% to about 20%, or by about 10% to about 30%. In some embodiments, an additional therapeutic agent is administered to the mammal. In some embodiments, the additional therapeutic agent is an anti-inflammatory agent, metabolic agent, or anti-fibrotic agent.

### Autoimmune Hepatitis

Autoimmune hepatitis is a chronic, autoimmune disease characterized by chronic liver inflammation and necrosis, which leads to cirrhosis. In some embodiments, an FXR agonist disclosed herein is used in the treatment of autoimmune hepatitis in a mammal. In some examples, an FXR agonist reduces autoimmune hepatitis in the mammal by at least 5%, at least 10%, at least 15%, at least 20%, at least 30%, at least 40%, at least 50%, or more. In some cases, autoimmune hepatitis is reduced by about 5% to about 50%, by about 5% to about 25%, by about 10% to about 20%, or by about 10% to about 30%. In some instances, the level of autoimmune hepatitis is relative to the level of autoimmune hepatitis in a mammal not treated with the FXR agonist. In some embodiments, an additional therapeutic agent is administered to the mammal. In some embodiments, the additional therapeutic agent is an anti-inflammatory agent, metabolic agent, or anti-fibrotic agent.

### Additional Liver Diseases or Conditions

In some embodiments, an FXR agonist disclosed herein is used to treat, prevent, or slow down the progression of end stage liver disease in a mammal. In some examples, an FXR agonist reduces end stage liver symptoms in the mammal by at least 5%, at least 10%, at least 15%, at least 20%, at least 30%, at least 40%, at least 50%, or more. In some instances, the end stage liver symptoms are reduced by about 5% to about 50%, by about 5% to about 25%, by about 10% to about 20%, or by about 10% to about 30%. In some instances, the progression of end stage liver disease is relative to the progression of end stage liver disease in a mammal not treated with an FXR agonist. In some embodiments, an additional therapeutic agent is administered to the mammal. In some embodiments, the additional therapeutic agent is an anti-inflammatory agent, metabolic agent, or anti-fibrotic agent.

Hepatocellular carcinoma is the most common type of liver cancer, often occurring in people with chronic liver diseases such as hepatitis B or hepatitis C. In many cases, FXR expression and signaling is downregulated in hepatocellular carcinoma patients. Given the role FXR plays in controlling bile acid metabolism, suppression of inflammatory signaling and enhancement of tissue repair, it is hypothesized that FXR plays a key role in preventing hepatocarcinogenesis. Additionally, studies have shown that treatment of hepatocellular carcinoma cells with FXR agonists results in inhibited cell growth. In some embodiments, an FXR agonist disclosed herein is used to treat hepatocellular carcinoma in a mammal. In some examples, an FXR agonist reduces hepatocellular carcinoma symptoms in the mammal by at least 5%, at least 10%, at least 15%, at least 20%, at least 30%, at least 40%, at least 50%, or more. In some instances, the hepatocellular carcinoma symptoms are reduced by about 5% to about 50%, by about 5% to about 25%, by about 10% to about 20%, or by about 10% to about 30%. In some instances, the progression of hepatocellular carcinoma is relative to the progression of hepatocellular carcinoma in a mammal not treated with an FXR agonist. In some embodiments, an additional therapeutic agent is administered to the mammal. In some embodiments, the additional therapeutic agent is an anti-inflammatory agent, metabolic agent, or anti-fibrotic agent.

In some embodiments, an FXR agonist disclosed herein reduce liver enzymes in a mammal. In some examples, an FXR agonist reduces liver enzymes (e.g., serum ALT and/or AST levels) in the mammal by at least 5%, at least 10%, at least 15%, at least 20%, at least 30%, at least 40%, at least 50%, or more. In some instances, the level of liver enzymes is reduced by about 5% to about 50%, by about 5% to about 25%, by about 10% to about 20%, or by about 10% to about 30%. In some instances, the level of liver enzymes is relative to the level of liver enzymes in a mammal not treated with an FXR agonist. In some embodiments, an additional therapeutic agent is administered to the mammal. In some embodiments, the additional therapeutic agent is an anti-inflammatory agent, metabolic agent, or anti-fibrotic agent.

In some embodiments, an FXR agonist disclosed herein reduce liver triglycerides in a mammal. In some examples, an FXR agonist reduces liver triglycerides in the mammal by at least 5%, at least 10%, at least 15%, at least 20%, at least 30%, at least 40%, at least 50%, or more. In some instances, the level of liver triglycerides is reduced by about 5% to about 50%, by about 5% to about 25%, by about 10% to about 20%, or by about 10% to about 30%. In some instances, the level of liver triglycerides is relative to the level of liver triglycerides in a mammal not treated with an FXR agonist. In some embodiments, an additional therapeutic agent is administered to the mammal. In some embodiments, the additional therapeutic agent is an anti-inflammatory agent, metabolic agent, or anti-fibrotic agent.

### Cholangiocarcinoma

Cholangiocarcinoma is a type of cancer that forms in the bile ducts of an individual. While it is not clear what causes the genetic mutations that lead to this cancer, risk factors include primary sclerosing cholangitis, chronic liver disease, and other bile duct problems. Inflammation and cholestasis are key factors in the formation of cholangiocarcinoma. Cholangiocarcinoma is classified by its location in the liver. Intrahepatic cholangiocarcinoma, the least common form of the dieses, begins in the small bile ducts within the liver. Perihilar cholangiocarcinoma (also called a Klatskin tumor) begins in the hilum, the region where two major bile ducts join and leave the liver. Perihilar cholangiocarcinoma is the most common form of the disease. The other form of cholangiocarcinoma is called distal cholangiocarcinoma, which begins in the bile ducts outside the liver.

Bile acids can activate epidermal growth factor receptor (EGFR) and enhance cyclooxygenase 2 (COX-2) expression. COX-2 dysregulates the growth of cholangiocarcinoma, enhances apoptosis-resistance, and positively regulates pro-oncogenic signaling pathways such as hepatocyte growth factor, IL-6, and EGFR, thus indicating a potential link between bile acid levels and incidence and progression of cholangiocarcinoma.

FXR expression is down-regulated in cholangiocarcinoma cells compared to healthy cholangiocytes. Studies have shown that treatment of cultures of human intrahepatic cholangiocarcinoma cells with the FXR agonist obeticholic acid can boost expression of FXR in vitro. Cholangiocarcinoma cells treated with the FXR agonist displayed decreased proliferation and increased apoptosis.

In some embodiments, an FXR agonist disclosed herein is used in the treatment of cholangiocarcinoma in a mammal. In some embodiments, the cholangiocarcinoma is intrahepatic cholangiocarcinoma. In some embodiments, the cholangiocarcinoma is perihilar cholangiocarcinoma. In some embodiments, the cholangiocarcinoma is distal cholangiocarcinoma. In some embodiments, treatment with the FXR agonist decreases proliferation of cholangiocarcinoma cells by at least 10%, at least 20%, at least 30%, at least 40%, or at least 50%. In some embodiments, treatment with the FXR agonist increases apoptosis of cholangiocarcinoma cells by at least 10%, at least 20%, at least 30%, at least 40%, or at least 50%. In some embodiments, treatment with the FXR agonist increases FXR expression in cholangiocarcinoma cells by at least 10%, at least 20%, at least 30%, at least 40%, or at least 50%.

In one aspect, described herein is a method of treating or preventing a liver disease or condition in a mammal, comprising administering to the mammal an FXR agonist disclosed herein, alone or in combination with other therapeutic agents. In some embodiments, the liver disease or condition is a fibrotic liver disease, a metabolic liver disease, an orphan liver disease, or any combination thereof.

### Gastrointestinal Diseases

Disclosed herein, in certain embodiments, are methods of treating or preventing a gastrointestinal disease in a subject in need thereof, comprising administering to the subject a farnesoid X receptor (FXR) agonist. In some embodiments, the gastrointestinal disease is associated with a liver disease. In some embodiments, the gastrointestinal disease is associated with a fibrotic liver disease. In some embodiments, the gastrointestinal disease is associated with a metabolic liver disease. In some embodiments, the gastrointestinal disease is irritable bowel syndrome (IBS), irritable bowel syndrome with diarrhea (IBS-D), irritable bowel syndrome with constipation (IBS-C), mixed IBS (IBS-M), unsubtyped IBS (IBS-U), or bile acid diarrhea (BAD). In some embodiments, the gastrointestinal disease is bile acid malabsorption, graft vs. host disease, Crohn's disease, inflammatory bowel disease, necrotizing enterocolitis, gastritis, ulcerative colitis, gastroenteritis, radiation induced enteritis, pseudomembranous colitis, chemotherapy induced enteritis, gastro-esophageal reflux disease (GERD), peptic ulcer, non-ulcer dyspepsia (NUD), celiac disease, intestinal celiac disease, post-surgical inflammation, gastrointestinal carcinogenesis, or any combination thereof.

### Irritable Bowel Syndrome

Irritable bowel syndrome (IBS) is a combination of symptoms including abdominal pain and changes in bowel movement patterns that persists over an extended period of time, often years. The causes of IBS remain unclear; however, gut motility problems, food sensitivity, genetic factors, small intestinal bacterial overgrowth, and gut-brain axis problems are thought to have a potential role. In some instances, IBS is accompanied with diarrhea and is categorized as IBS with diarrhea (IBS-D). In some instances, IBS is accompanied with constipation and is categorized as IBS with constipation (IBS-C). In some instances, IBS is accompanied with an alternating pattern of diarrhea and constipation and is categorized as mixed IBS (IBS-M). In some instances, IBS is not accompanied with either diarrhea or constipation and is categorized as unsubtyped IBS (IBS-U). In some instances, IBS has four different variations: IBS-D, IBS-C, IBS-M, and IBS-U.

In some embodiments, the symptoms of IBS are mimicked by a different condition. In some embodiments, sugar maldigestion, celiac disease, gluten intolerance without celiac disease, pancreatic exocrine insufficiency, small bowel bacterial overgrowth, microscopic colitis, or bile acid malabsorption (BAM) mimic IBS-D. In some embodiments, anismus, pelvic floor dyssynergia or puborectalis spasm, or descending perineum syndrome mimic IBS-C. In some embodiments, certain conditions contribute to the symptoms of patients that have IBS. In some embodiments, certain conditions are the major contributors of the symptoms in patients that have IBS. In some embodiments, non-limiting examples of these conditions are: sugar maldigestion, celiac disease, gluten intolerance without celiac disease, pancreatic exocrine insufficiency, small bowel bacterial overgrowth, microscopic colitis, bile acid malabsorption (BAM), anismus, pelvic floor dyssynergia or puborectalis spasm, or descending perineum syndrome mimic IBS-C.

In some embodiments, an FXR agonist disclosed herein is used in combination with another therapeutic agent as disclosed herein in the treatment of IBS or any of its variations in a mammal. In some examples, an FXR agonist reduces symptoms caused by IBS or any of its variations in the mammal by at least 5%, at least 10%, at least 15%, at least 20%, at least 30%, at least 40%, at least 50%, or more. In some cases, IBS or any of its variations is reduced by about 5% to about 50%, by about 5% to about 25%, by about 10% to about 20%, or by about 10% to about 30%. In some embodiments, an additional therapeutic agent is administered to the mammal. In some embodiments, the additional therapeutic agent is an anti-inflammatory agent, metabolic agent, or anti-fibrotic agent.

### Bile Acid Malabsorption

Bile acid malabsorption (BAM), also known as bile acid diarrhea (BAD), bile acid-induced diarrhea, cholerheic or choleretic enteropathy, or bile salt malabsorption, is a condition in which the presence of bile acids in the colon causes diarrhea. BAM is caused by a number of conditions such as Crohn's disease, cholecystectomy, coeliac disease, radiotherapy, and pancreatic diseases. In some instances, BAM is idiopathic. In some instances, BAM is caused by medications such as metformin. In some embodiments, BAM is caused by an overproduction of bile acids. Bile acid synthesis is negatively regulated by the ileal hormone fibroblast growth factor 19 (FGF-19); low levels of FGF-19 lead to an increase in bile acids. FXR activation promotes the synthesis of FGF-19, consequently lowering the levels of bile acids.

In some embodiments, an FXR agonist disclosed herein is used in combination with another therapeutic agent as disclosed herein in the treatment of BAM in a mammal. In some embodiments, an FXR agonist disclosed herein is used in combination with another therapeutic agent as disclosed herein to decrease bile acid synthesis. In some embodiments, an FXR agonist disclosed herein to decrease bile acid levels. In some embodiments, an FXR agonist disclosed herein is used in combination with another therapeutic agent as disclosed herein to prevent BAM. In some examples, an FXR agonist disclosed herein is used in combination with another therapeutic agent as disclosed herein to reduce BAM symptoms in the mammal by at least 5%, at least 10%, at least 15%, at least 20%, at least 30%, at least 40%, at least 50%, or more. In some cases, BAM is reduced by about 5% to about 50%, by about 5% to about 25%, by about 10% to about 20%, or by about 10% to about 30%. In some embodiments, an additional therapeutic agent is administered to the mammal. In some embodiments, the additional therapeutic agent is an anti-inflammatory agent, metabolic agent, or anti-fibrotic agent.

Graft vs. Host Disease (GvHD)

Graft vs. host disease (GvHD) is a medical complication that arises after a transplant of tissue or cells from a histo-incompatible donor (i.e. a genetically or immunologically different donor). Immune cells in the donated tissue or cells (graft) recognize the recipient (the host) as foreign and initiate an immune attack. Non-limiting examples of transplanted tissue or cells that give rise to GvHD are blood products, stem cells such as bone marrow cells, and organs. There are different types of GvHD depending on where the symptoms manifest or develop; for example, skin GvHD, liver GvHD, eye GvHD, neuromuscular GvHD, genitourinary tract GvHD, and gastrointestinal (GI) tract GvHD. Symptoms of GI tract GvHD include difficulty swallowing, pain with swallowing, weight loss, nausea, vomiting, diarrhea, and/or abdominal cramping. GI tract GvHD results in sloughing of the mucosal membrane and severe intestinal inflammation. Inflammation of the biliary epithelium is amenable to be controlled by nuclear receptors such as the glucocorticoid receptor (GR), FXR, or the peroxisome proliferator-activated receptors (PPARs).

In some embodiments, an FXR agonist disclosed herein is used in combination with another therapeutic agent as disclosed herein in the treatment of GvHD or a complication of GvHD in a mammal. In some embodiments, an FXR agonist disclosed herein is used in combination with another therapeutic agent as disclosed herein in the treatment of GI tract GvHD or a complication of GI tract GvHD in a mammal. In some examples, an FXR agonist disclosed herein is used in combination with another therapeutic agent as disclosed herein to reduce GI tract GvHD or a complication of GI tract GvHD in the mammal by at least 5%, at least 10%, at least 15%, at least 20%, at least 30%, at least 40%, at least 50%, or more. In some cases, GI tract GvHD or a complication of GI tract GvHD is reduced by about 5% to about 50%, by about 5% to about 25%, by about 10% to about 20%, or by about 10% to about 30%. In some embodiments, an FXR agonist disclosed herein is used in combination with another therapeutic agent as disclosed herein to decrease intestinal inflammation caused by GI tract GvHD. In some embodiments, an FXR agonist disclosed herein reduce intestinal inflammation caused by GI tract GvHD reduced by about 5% to about 50%, by about 5% to about 25%, by about 10% to about 20%, or by about 10% to about 30%. In some embodiments, the additional therapeutic agent is an anti-inflammatory agent, metabolic agent, or anti-fibrotic agent.

Inflammatory bowel disease (IBD) is an autoimmune disease characterized by a set of inflammatory conditions that affect the colon and the small intestine. Ulcerative colitis and Crohn's disease are the main types of inflammatory bowel diseases. FXR activation is decreased in patients with IBD. Increasing FXR activity via the administration of FXR agonists disclosed herein, alone or in combination with the additional therapeutic agents disclosed herein prevents and/or decreases the symptoms of IBD. Increasing FXR activity via the administration of FXR agonists disclosed herein, alone or in combination with the additional therapeutic agents disclosed herein reduces intestinal inflammation in IBD patients.

In some embodiments, an FXR agonist disclosed herein is used in combination with another therapeutic agent as disclosed herein in the treatment of IBD in a mammal. In some embodiments, an FXR agonist disclosed herein is used in combination with another therapeutic agent as disclosed herein to decrease intestinal inflammation. In some embodiments, an FXR agonist disclosed herein is used in combination with another therapeutic agent as disclosed herein to prevent IBD. In some examples, an FXR agonist disclosed herein is used in combination with another therapeutic agent as disclosed herein to reduce IBD symptoms in the mammal by at least 5%, at least 10%, at least 15%, at least 20%, at least 30%, at least 40%, at least 50%, or more. In some cases, IBD is reduced by about 5% to about 50%, by about 5% to about 25%, by about 10% to about 20%, or by about 10% to about 30%. In some embodiments, the additional therapeutic agent is an anti-inflammatory agent, metabolic agent, or anti-fibrotic agent.

In one aspect, described herein is a method of treating or preventing a gastrointestinal disease or condition in a mammal, comprising administering to the mammal an FXR agonist disclosed herein in combination with a compound of Formula (I), Formula (II), Formula (IV), or Formula (VII), or a pharmaceutically acceptable salt or solvate thereof. In some embodiments, the gastrointestinal disease or condition is necrotizing enterocolitis, gastritis, ulcerative colitis, Crohn's disease, inflammatory bowel disease, irritable bowel syndrome, gastroenteritis, radiation induced enteritis, pseudomembranous colitis, chemotherapy induced enteritis, gastro-esophageal reflux disease (GERD), peptic ulcer, non-ulcer dyspepsia (NUD), celiac disease, intestinal celiac disease, post-surgical inflammation, gastric carcinogenesis, graft versus host disease, or any combination thereof. In some embodiments, the gastrointestinal disease or condition is inflammatory bowel disease.

### Gastrointestinal Cancers

FXR is predominantly expressed in tissues exposed to high levels of bile acids, such as the entire gastrointestinal tract, the liver, the bile duct and gallbladder. Recent observations note that a fat-rich diet is positively associated with colon cancer incidence. Consumption of high-fat diet has been correlated with elevated levels of bile acids in the colonic lumen as a consequence of increased fecal excretion of bile acids. Subjects who consume a western diet display elevated levels of fecal secondary bile acids, as do patients diagnosed with colonic carcinomas. Elevated secondary bile acid concentrations have detrimental effects on colonic epithelium architecture and function through multiple mechanisms, such as DNA oxidative damage, inflammation, NF-κB activation and enhanced cell proliferation. As a result, bile acids can be considered as tumor-promoting factors in colorectal cancer development.

In some embodiments, a FXR agonist (e.g. Compound 1, or a pharmaceutically acceptable salt thereof), is used in the treatment of a gastrointestinal cancer. In some embodiments, a FXR agonist (e.g. Compound 1, or a pharmaceutically acceptable salt thereof), is used in the treatment of a gastrointestinal cancer in combination with an additional therapeutic agent. In some embodiments, a FXR agonist (e.g. Compound 1, or a pharmaceutically acceptable salt thereof), slows or prevents the progression of a gastrointestinal cancer through activation of FXR.

In some embodiments, the gastrointestinal cancer is anal cancer, colon cancer, esophageal cancer, gallbladder cancer, biliary tract cancer, liver cancer, cholangiocarcinoma, pancreatic cancer, peritoneal cancer, rectal cancer, colorectal cancer, small intestine cancer, stomach (gastric) cancer, gastro-intestinal stromal tumour (GIST), neuroendocrine tumours (NETs), or small bowel cancer. In some embodiments, the gastrointestinal cancer is colorectal cancer.

### Kidney Diseases

Disclosed herein, in certain embodiments, are methods of treating a kidney disease in a subject in need thereof, comprising administering to the subject a farnesoid X receptor (FXR) agonist and an additional therapeutic agent. In some embodiments, the kidney disease is associated with a liver disease. In some embodiments, the kidney disease is associated with a fibrotic liver disease. In some embodiments, the kidney disease is associated with a metabolic liver disease. In some embodiments, the kidney disease is associated with a metabolic condition such as but not limited to diabetes, metabolic syndrome, NAFLD, insulin resistance, fatty acid metabolism disorder, and cholestasis. In some embodiments, the kidney disease is diabetic nephropathy, kidney disease associated with fibrosis, kidney disease not associated with fibrosis, renal fibrosis, or any combination thereof. In some embodiments, the kidney disease is associated with tubulointerstitial nephritis/nephropathy. In some embodiments, the kidney disease is associated with glomerulonephritis/nephropathy.

In some embodiments, a FXR agonist (e.g. Compound 1, or a pharmaceutically acceptable salt thereof), is used in the treatment of tubulointerstitial nephritis/nephropathy and/or glomerulonephritis/nephropathy. In some embodiments, a FXR agonist (e.g. Compound 1, or a pharmaceutically acceptable salt thereof), is used in the treatment of tubulointerstitial nephritis/nephropathy. In some embodiments, a FXR agonist (e.g. Compound 1, or a pharmaceutically acceptable salt thereof), is used in the treatment of glomerulonephritis/nephropathy.

In some embodiments, the tubulointerstitial nephritis/nephropathy is drug-induced tubulointerstitial nephritis/nephropathy, toxin-induced tubulointerstitial nephritis, radiation-induced tubulointerstitial nephritis, ischemia-induced tubulointerstitial nephritis, or idiopathic tubulointerstitial nephritis.

In some embodiments, the glomerulonephritis/nephropathy is an IgA nephropathy, focal segmental glomerulsclerosis, minimal change glomerulonephritis, drug-induced glomerulonephritis, infection-induced (post-strep) glomerulonephritis, vasculitis-induced glomerulonephritis, or glomerulonephritis secondary to systemic diseases, including but not limited to amyloidosis and systemic lupus erythematosus.

### Diabetic Nephropathy

In some embodiments, factors contributing to kidney disease include hyperlipidemia, hypertension, hyperglycemia, and proteinuria, all of which result in further damage to the kidneys and further stimulate the extracellular matrix deposition. Furthermore, dysregulation of glucose results in the stimulation of cytokine release and upregulation of extracellular matrix deposition. Regardless of the primary cause, insults to the kidneys may result in kidney fibrosis and the concomitant loss of kidney function.

Diabetic nephropathy is a kidney disease characterized by damage to the kidney's glomeruli. Diabetes contributes to an excessive production of reactive oxygen species, which leads to nephrotic syndrome and scarring of the glomeruli. As diabetic nephropathy progresses, the glomerular filtration barrier (GFB) is increasingly damaged and consequently, proteins in the blood leak through the barrier and accumulate in the Bowman's space.

In some embodiments, an FXR agonist disclosed herein is used in combination with another therapeutic agent as disclosed herein in the treatment of diabetic nephropathy in a mammal. In some examples, an FXR agonist and an additional therapeutic agent reduce diabetic nephropathy symptoms in the mammal by at least 5%, at least 10%, at least 15%, at least 20%, at least 30%, at least 40%, at least 50%, or more. In some cases, diabetic nephropathy is reduced by about 5% to about 50%, by about 5% to about 25%, by about 10% to about 20%, or by about 10% to about 30%. In some embodiments, the additional therapeutic agent is an anti-inflammatory agent, metabolic agent, or anti-fibrotic agent.

### Renal Fibrosis

Renal fibrosis is characterized by activation of fibroblasts and excessive deposition of extracellular matrix or connective tissue in the kidney, which is a hallmark of chronic kidney disease. FXR plays an important role in protecting against renal fibrosis. Activation of FXR suppresses renal fibrosis and decreases accumulation of extracellular matrix proteins in the kidney.

In some embodiments, a FXR agonist (e.g. Compound 1, or a pharmaceutically acceptable salt thereof), is used in the treatment of disease or condition associated with kidney fibrosis. Kidney fibrosis can result from various diseases and insults to the kidneys. Examples of such diseases and insults include chronic kidney disease, metabolic syndrome, vesicoureteral reflux, tubulointerstitial renal fibrosis, IgA nephropathy, diabetes (including diabetic nephropathy), Alport syndrome, HIV associated nephropathy, resultant glomerular nephritis (GN), including, but not limited to, focal segmental glomerulosclerosis and membranous glomerulonephritis, mesangiocapillary GN and resultant interstitial fibrosis and tubular atrophy (IFTA), including but not limited to, recovery post acute kidney injury (AKI), acute obstructive nephropathy and drug induced kidney fibrosis.

In some embodiments, an FXR agonist disclosed herein is used in combination with another therapeutic agent as disclosed herein in the treatment of renal fibrosis in a mammal. In some examples, an FXR agonist and an additional therapeutic agent reduce renal fibrosis symptoms in the mammal by at least 5%, at least 10%, at least 15%, at least 20%, at least 30%, at least 40%, at least 50%, or more. In some cases, renal fibrosis is reduced by about 5% to about 50%, by about 5% to about 25%, by about 10% to about 20%, or by about 10% to about 30%. In some embodiments, the additional therapeutic agent is an anti-inflammatory agent, metabolic agent, or anti-fibrotic agent.

In one aspect, described herein is a method of treating or preventing a kidney disease or condition in a mammal, comprising administering to the mammal an FXR agonist disclosed herein in combination with a compound of Formula (I), Formula (II), Formula (IV), or Formula (VII), or a pharmaceutically acceptable salt or solvate thereof. In some embodiments, the kidney disease or condition is diabetic nephropathy, kidney disease associated with fibrosis, kidney disease not associated with fibrosis, renal fibrosis, kidney disease associated with a metabolic disease, chronic kidney disease, polycystic kidney disease, acute kidney disease, or any combination thereof.

### Inflammation

Disclosed herein, in certain embodiments, are methods of treating or preventing inflammation in a subject in need thereof, comprising administering to the subject a farnesoid X receptor (FXR) agonist and an additional therapeutic agent. In some embodiments, the additional therapeutic agent is an anti-fibrotic therapeutic agent, anti-inflammatory agent, a metabolic therapeutic agent, an anti-inflammatory agent, or any of the other therapeutic agents described herein.

In some embodiments, the inflammation is liver inflammation. In some embodiments, the liver inflammation is acute hepatitis, chronic hepatitis, or fulminant hepatitis. In some embodiments, the liver inflammation is viral hepatitis, bacterial hepatitis, parasitic hepatitis, toxic- and drug-induced hepatitis, alcoholic hepatitis, autoimmune hepatitis, non-alcoholic steatohepatitis (NASH), neonatal hepatitis, or ischemic hepatitis. In some embodiments, the viral hepatitis is viral hepatitis is hepatitis A, hepatitis B, hepatitis C, hepatitis D, or hepatitis E. In some embodiments, the liver inflammation is accompanied by a fibrotic liver disease or a metabolic liver disease.

In some embodiments, an FXR agonist disclosed herein is used in combination with another therapeutic agent as disclosed herein in the treatment of inflammation or an inflammatory condition in a mammal. In some examples, an FXR agonist disclosed herein is used in combination with another therapeutic agent as disclosed herein to reduce symptoms of inflammation or an inflammatory condition in the mammal by at least 5%, at least 10%, at least 15%, at least 20%, at least 30%, at least 40%, at least 50%, or more. In some cases, inflammation or an inflammatory condition is reduced by about 5% to about 50%, by about 5% to about 25%, by about 10% to about 20%, or by about 10% to about 30%. In some embodiments, the additional therapeutic agent is an anti-inflammatory agent, metabolic agent, or anti-fibrotic agent.

In one aspect, described herein is a method of treating or preventing inflammation or an inflammatory condition in a mammal, comprising administering to the mammal an FXR agonist disclosed herein in combination with a compound of Formula (I), Formula (II), Formula (IV), or Formula (VII), or a pharmaceutically acceptable salt or solvate thereof. In some embodiments, inflammation or the inflammatory condition is liver inflammation, kidney inflammation, gastrointestinal inflammation, or any combination thereof.

### FXR Agonists

In some embodiments, the FXR agonists used in the disclosed methods to treat or prevent a liver disease, a gastrointestinal disease, a metabolic liver disease, or a fibrotic liver disease are the compounds described herein. In one aspect, the compounds described herein include pharmaceutically acceptable salts, prodrugs, active metabolites, and pharmaceutically acceptable solvates thereof. In some embodiments, the FXR agonist is a non-bile acid, bile acid analog, or other natural FXR ligand.

In some embodiments, the FXR agonist for use in any of the embodiments described herein is a compound described in International Application No. PCT/US2015/020582, filed on 13 Mar. 2015; application Ser. No. 15/263,048, filed on 12 Sep. 2016; International Application No. PCT/US2015/020552, filed on 13 Mar. 2015; application Ser. No. 15/263,033, filed on 12 Sep. 2016; International Application No. PCT/US2016/052268, filed on 16 Sep. 2016; International Application No. PCT/US2016/052274, filed on 16 Sep. 2016; International Application No. PCT/US2016/052275, filed on 16 Sep. 2016; International Application No. PCT/US2016/052270, filed on 16 Sep. 2016; International Application No. PCT/US2018/022488 filed on 14 Mar. 2018; International Application No. PCT/US2018/022489 filed on 14 Mar. 2018; International Application No. PCT/US2018/022497 filed on 14 Mar. 2018; International Application No. PCT/US2018/022513 filed on 14 Mar. 2018.

In some embodiments, the FXR agonist for use in any of the embodiments described herein is a compound that has the following structure of Compound 1:

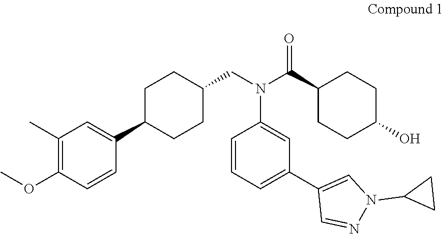 [SMILES: COc1ccc([C@H]2CC[C@H](CN(c3cccc(-c4cnn(C5CC5)c4)c3)C(=O)[C@H]3CC[C@H](O)CC3)CC2)cc1C]

or a pharmaceutically acceptable salt thereof.

In some embodiments, the FXR agonist is obeticholic acid, a chenodeoxycholic acid, or a non-bile acid FXR agonist. In some embodiments, the non-bile acid FXR agonist is NTX023-1 (Ardelyx), AKN-083 (Allergan), LJN-452 (Novartis), or EDP-305 (Enanta Pharmaceuticals). In some embodiments, the non-bile acid FXR agonist is a natural or synthetic non-steroidal agonist. In some embodiments, the synthetic non-steroidal agonist is 4-(2-(2-chloro-4-((5-cyclopropyl-3-(2,6-dichlorophenyl)isoxazol-4-yl)methoxy)phenyl)cyclopropyl)benzoic acid or 3-(2,6-dichlorophenyl)-4-(3′-carboxy-2-chlorostilben-4-yl)oxymethyl-5-isopropylisoxazole. In some embodiments, the FXR agonist is GS9674 (Gilead). In some embodiments, the FXR agonist is fexaramine.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

The term “acceptable” with respect to a formulation, composition or ingredient, as used herein, means having no persistent detrimental effect on the general health of the mammal being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that is used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that are employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein, or a pharmaceutically acceptable salt thereof, and a co-agent, are both administered to a patient simultaneously in the form of a single entity or dosage. The term “non-fixed combination” means that the active ingredients, e.g. a compound described herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, halting progression of the disease or condition, relieving a condition caused by the disease or condition, or stopping the symptoms of the disease or condition either prophylactically and/or therapeutically.

The terms “about” or “approximately” mean within an acceptable error range for the particular value as determined by one of ordinary skill in the art, which will depend in part on how the value is measured or determined, e.g., the limitations of the measurement system. Where particular values are described in the application and claims, unless otherwise stated the term “about” should be assumed to mean an acceptable error range for the particular value.

### Combination Therapies

In any of the embodiments described herein, a FXR agonist (e.g. Compound 1, or a pharmaceutically acceptable salt thereof), is used with any additional therapeutic agent. In any of the embodiments described herein, the FXR agonist is used with any additional therapeutic agent as described herein. For example, in some embodiments, the additional therapeutic agent is a small molecule, macromolecule, an oligonucleotide, a virus, bacteria, an anti-inflammatory agent, an immunomodulatory agent, an anti-cancer agent, weight loss, an agent to treat NASH, an agent to treat diabetes, an agent to treat insulin resistance, a statin, an insulin sensitizing agent, a vitamin, an anti-fungal agent, an antioxidant, a corticosteroid, an anti-tumor necrosis factor (TNF) agent, an antibiotic, a chemotherapeutic agent, a biologic agent, a radiotherapeutic agent, an anti-obesity agent, a nutraceutical, radiation therapy, or an agent to treat primary biliary cholangitis.

In some embodiments, an FXR agonist is administered together with a modulator of any one of the following target proteins: cannabinoid receptor 1, cannabinoid receptor 2, peroxisome proliferator-activated receptor (PPAR)-delta, PPAR gamma, PPAR alpha, PPAR alpha and PPAR delta (dual modulation), smoothened (SMO), Hedgehog signaling effectors such as Gli-1 and Gli-2, Yes-associated protein (YAP), transcriptional coactivator with PDZ-binding motif (TAZ), heat shock protein 47 (HSP47), collagen type 1 alpha 1 (COL1A1), transforming growth factor (TGF)-beta, alpha-5 beta-6 integrin, platelet-derived growth factor (PDGF), apical sodium-bile acid transporter (ASBT), C-C chemokine receptor type 2 (CCR2), C-C chemokine receptor type 5 (CCR5), dual C-C chemokine receptor type 2/C-C chemokine receptor type 5 (CCR 2/5), lysophosphatidic acid receptor (LPA)-1, autotaxin, apoptosis signal-regulating kinase 1 (ASK1), NADPH oxidase 1 (NOX1), NADPH oxidase 4 (NOX4), NADPH oxidase 2 (NOX2), NADPH oxidase 5 (NOX5), dual oxidase 1 (DUOX1), dual oxidase 2 (DUOX2), caspase, galectin 3, pentraxin-2, acetyl CoA carboxylase, glucagon-like peptide-1 (GLP-1), inducible nitric oxide synthase (iNOS), N-acetylcysteine, S-adenosyl-methionine, lysyl oxidase (LOXL2), antiogensin 2 receptor, bromodomain containing 4 (BRD4), eukaryotic translation initiation factor 4E (eIF4E), vascular endothelial growth factor (VEGF), fibroblast activation protein, vitamin D receptor, toll-like receptor 4 (TLR4), TIMP metallopeptidase inhibitor 1 (TIMP-1), C-X-C chemokine receptor type 3 (CXCR3), interleukin-13 (IL-13), IL-4, alpha v beta 3 integrin, fibroblast growth factor 19, fibroblast growth factor 21, ABCA1/SCD1, Thyroid hormone receptor (THR) β, diacylglycerol acyltransferase 1 (DGAT-1), diacylglycerol acyltransferase 2 (DGAT-2), discoidin domain receptor 1 (DDR1), discoidin domain receptor (DDR2), focal adhesion kinase (FAK), semicarbazide-sensitive amine oxidase (SSAO/VAP-1), 17b-HSD type 13, GPR84, protease activated receptor (PAR-2), or retinoic acid receptor-Related orphan receptor γt (RORγt).

In some embodiments, a FXR agonist (e.g. Compound 1, or a pharmaceutically acceptable salt thereof), is administered in combination with a modulator of any one of the following target proteins: cannabinoid receptor 1, cannabinoid receptor 2, peroxisome proliferator-activated receptor (PPAR)-delta, PPAR gamma, PPAR alpha, PPAR alpha and PPAR delta (dual modulation), heat shock protein 47 (HSP47), fibroblast growth factor 19, fibroblast growth factor 21, transforming growth factor (TGF)-beta, apical sodium-bile acid transporter (ASBT), ABCA1/SCD1, C-C chemokine receptor type 2 (CCR2), C-C chemokine receptor type 5 (CCR5), dual C-C chemokine receptor type 2/C-C chemokine receptor type 5 (CCR 2/5), lysophosphatidic acid receptor (LPA)-1, autotaxin, apoptosis signal-regulating kinase 1 (ASK1), caspase, acetyl CoA carboxylase (ACC), glucagon-like peptide-1 (GLP-1), N-acetylcysteine, S-adenosyl-methionine, lysyl oxidase (LOXL2), antiogensin 2 receptor, vascular endothelial growth factor (VEGF), fibroblast activation protein, Thyroid hormone receptor (THR) β, diacylglycerol acyltransferase 1 (DGAT-1), diacylglycerol acyltransferase 2 (DGAT-2), discoidin domain receptor 1 (DDR1), discoidin domain receptor (DDR2), focal adhesion kinase (FAK), semicarbazide-sensitive amine oxidase (SSAO/VAP-1), 17b-HSD type 13, GPR84, protease activated receptor (PAR-2), retinoic acid receptor-related orphan receptor γt (RORγt)

In any of the embodiments described herein, the additional therapeutic agent is an agent used to treat a metabolic disease or condition. In any of the embodiments described herein, the additional therapeutic agent is an agent used to treat a fibrotic disease or condition. In some embodiments, the additional therapeutic agent used to treat a fibrotic disease or condition is pirfenidone.

In some embodiments, the additional therapeutic agent that is administered in conjunction with an FXR agonist as part as a method of treating or preventing a liver disease, including but not limited to a fibrotic liver disease or a metabolic liver disease, in a subject in need thereof is an anti-fibrotic therapeutic agent, anti-inflammatory agent, or a metabolic therapeutic agent.

In some embodiments, a FXR agonist (e.g. Compound 1, or a pharmaceutically acceptable salt thereof), is administered in combination with a cannabinoid receptor 1 antagonist, a smoothened receptor (SMO) antagonist, a Yes-associated protein (YAP), a PDZ-binding motif (TAZ) antagonist, a heat shock protein 47 (HSP47) antagonist, a collagen type 1 alpha 1 (COL1a1) antagonist, a transforming growth factor-β (TGF-(3) antagonist, an alpha-5 beta-6 integrin antagonist, pirfenidone, a platelet-derived growth factor (PDGF) antagonist, a C-C chemokine receptor type 2 and 5 (CCR2/CCR5) antagonist, a lysophosphatidic acid receptor11 (LPA-1) antagonist, an autotaxin antagonist, an apoptosis signal-regulating kinase 1 (ASK1) antagonist, glucagon-like peptide-1 (GLP-1) agonist, peroxisome proliferator-activated receptor (PPAR)-delta agonist, PPAR gamma agonist, PPAR alpha agonist, PPAR alpha and PPAR delta dual agonist, acetyl CoA carboxylase (ACC) inhibitor, fibroblast growth factor 19 analogue, fibroblast growth factor 21 analogue, ABCA1/SCD1 modulator, thyroid hormone receptor (THR) β agonist, diacylglycerol acyltransferase 1 (DGAT-1) inhibitor, diacylglycerol acyltransferase 2 (DGAT-2) inhibitor, discoidin domain receptor 1 (DDR1) inhibitor, discoidin domain receptor (DDR2) inhibitor, focal adhesion kinase (FAK) inhibitors, semicarbazide-sensitive amine oxidase (SSAO/VAP-1) inhibitor, 17b-HSD type 13 inhibitor, GPR84 antagonist, protease activated receptor (PAR-2) antagonist, or retinoic acid receptor-related orphan receptor γt (RORγt) antagonist/inverse agonist. an NADPH oxidase 1 (NOX1) antagonist, NOX2 antagonist, a dual NOX1/NOX4 antagonist, a NOX5 antagonist, a DUOX1 antagonist, a DUOX2 antagonist, a NOX4 antagonist, a caspase antagonist, a galectin 3 antagonist, an inducible nitric oxide synthase (iNOS) antagonist, N-acetylcysteine, a Lysyl oxidase homolog 2 (LOXL2) antagonist, an angiotensin 2 receptor antagonist, a bromodomain-containing protein 4 (BRD4) inhibitor, a eukaryotic translation initiation factor-4E (eIF4E) antagonist, a cannabinoid receptor 2 agonist, a vascular endothelial growth factor (VEGF) agonist, a VEGF antagonist, a fibroblast activation protein antagonist, a vitamin D receptor antagonist, a toll-like receptor 4 (TLR4) antagonist, a tissue inhibitor of metalloproteinase-1 (TIMP-1) antagonist, ursodiol, or nonursodiol.

Combination with a Chemokine Receptor (CCR) Inhibitor

The recruitment of inflammatory monocytes and macrophages via chemokine receptor type 2 (CCR2) as well as of lymphocytes and hepatic stellate cells via chemokine receptor type 5 (CCR5) promote the progression of NASH to fibrosis.

Obesity-associated macrophage infiltration of adipose and hepatic tissue is mediated by chemokine receptor type 2 (CCR2), in which CCR2-positive, CD11b-positive, F4/80-positive macrophages contribute to chronic inflammation and insulin resistance.

Several studies have emphasized the importance of CCR2 and CCR5 in inflammation and fibrosis. In some embodiments, inhibitors of CCR2 and/or CCR5 improve insulin sensitivity and glucose tolerance compared with control subjects, reduce ALT concentrations and hepatic triglyceride content, improve insulin sensitivity, or combinations thereof.

In some embodiments, a FXR agonist (e.g. Compound 1, or a pharmaceutically acceptable salt thereof), is administered in combination with a CCR inhibitor. In some embodiments, the CCR inhibitor is a CCR2 inhibitor, a CCR5 inhibitor, or a dual inhibitor of CCR2 and CCR5.

In some embodiments, a FXR agonist (e.g. Compound 1, or a pharmaceutically acceptable salt thereof), is administered in combination with a CCR2 inhibitor, a CCR5 inhibitor, or a dual inhibitor of CCR2 and CCR5. In some embodiments, a FXR agonist (e.g. Compound 1, or a pharmaceutically acceptable salt thereof), is administered in combination with a CCR2 inhibitor. In some embodiments, the CCR2 inhibitor is CCX872. In some embodiments, a FXR agonist (e.g. Compound 1, or a pharmaceutically acceptable salt thereof), is administered in combination with a dual inhibitor of CCR2 and CCR5. In some embodiments, the dual inhibitor of CCR2 and CCR5 is cenicriviroc (Tobira Therapeutics).

Combination with a Caspase Inhibitor

Caspases are a family of related enzymes that play an important role as modulators of critical cellular functions, including functions that result in apoptosis and inflammation. Caspase activation and regulation is tightly controlled through a number of mechanisms. All caspases are expressed as enzymatically inactive forms known as pro-caspases, which can be activated following a variety of cellular insults or stimuli. Seven caspases are specifically involved in the process of apoptosis while three caspases specifically activate pro-inflammatory cytokines and are not directly involved in apoptosis.

Caspase mediated apoptosis is driven primarily by the activity of caspases 3 and 7 which, by virtue of their enzymatic activity, cleave a wide variety of cellular proteins and result in dismantling of the cell. Other apoptotic caspase family members are principally involved in sensing and transmitting signals from either outside or inside the cell. These signals converge to activate pro-caspases 3 and 7, enabling them to carry out the process of apoptosis.

While healthy individuals have normal levels of apoptosis, excessive levels of apoptosis associated with disease can overwhelm the body's normal clearance mechanisms. Reducing excessive levels of apoptosis reestablishes balance between apoptotic activity and normal clearance mechanisms and brings inflammation and other drivers of disease progression under control. As a result, targeting caspases that drive both apoptosis and inflammation in disease offers a unique and potentially powerful therapeutic approach for the treatment of both acute and chronic liver disease.

In some embodiments, a FXR agonist (e.g. Compound 1, or a pharmaceutically acceptable salt thereof), is administered in combination with a caspase inhibitor. In some embodiments, the caspase inhibitor is emricasan.

Combination with a Cannabinoid Receptor 1 (CB1) Modulator

CB1 is a guanine-nucleotide-binding protein (G-protein) coupled receptor located mainly in neuronal cells, but also in peripheral tissues. CB1 plays an important role in neurotransmission, embryonic development, and metabolism. CB1 is a principal mediator of insulin resistance and liver lipogenesis. CB1 has also been associated with hepatic fibrosis. In healthy subjects, the expression levels of CB1 in the liver are absent or low. In contrast, the expression of CB1 is upregulated in liver diseases. Activation of CB1 elicits pro-fibrogenic and pro-inflammatory effects in liver tissue. Blocking, inhibiting, decreasing, or dampening CB1's activity provides a method of treating or preventing a liver disease in a subject in need thereof.

In some embodiments, a FXR agonist (e.g. Compound 1, or a pharmaceutically acceptable salt thereof), is administered in combination with a CB1 modulator. In some embodiments, the CB1 modulator is CB1 antagonist, or CB1 inverse agonist.

In some embodiments, the CB1 modulator is nimacimab (Bird Rock Bio).

In some embodiments, the cannabinoid receptor 1 inverse agonist is JD5037 (Jenrin; (E)-N2-([(4S)-3-(4-chlorophenyl)-4-phenyl-4,5-dihydro-1H-pyrazol-1-yl]{[(4-chlorophenyl)sulfonyl]amino}methylene)-L-valinamide.

In some embodiments, the cannabinoid receptor 1 inverse agonist is TM38837 (7TM Pharma; 1-(2,4-dichlorophenyl)-4-ethyl-N-(1-piperidinyl)-5-(5-{[4-(trifluoromethyl)phenyl]ethynyl}-2-thienyl)-1H-pyrazole-3-carboxamide).

In some embodiments, the cannabinoid receptor 1 inverse agonist is MRI-1867 (Inversago). In some embodiments, an FXR agonist is administered to a subject in need thereof in combination with MRI-1867.

In some embodiments, the cannabinoid receptor 1 inverse agonist is AM6545 (MAK Scientific; 5-(4-[4-cyanobut-1-ynyl]phenyl)-1-(2,4-dichlorophenyl)-4-methyl-N-(1,1-dioxo-thiomorpholino)-1H-pyrazole-3-carboxamide).

In some embodiments, the CB1 modulator is a peripheral antagonist antibody to cannabinoid receptor 1 (CB1). In some embodiments, the peripheral antagonist antibody to cannabinoid receptor 1 (CB1) is nimacimab (Bird Rock Bio). In some embodiments, an FXR agonist is administered to a subject in need thereof in combination with nimacimab. In some embodiments, a therapeutically effective dose of nimacimab is administered to a subject in need thereof. In some embodiments, nimacimab is administered orally, transdermally, or via an intravenous, intramuscular, subcutaneous, or intraperitoneal injection.

Combination with a Cannabinoid Receptor 2 (CB2) Agonist

CB2 is a guanine-nucleotide-binding protein (G-protein) coupled receptor that is closely related to CB1 and is located in peripheral tissues of the immune system, brain, gastrointestinal tissues, and in mast cells. CB2 plays an important role in inflammation, pain, atherosclerosis, and osteoporosis. CB2 has also been associated with chronic and acute liver injury, including fibrogenesis associated to chronic liver diseases, ischaemia-reperfusion-induced liver injury, and hepatic encephalopathy associated to acute liver failure. Activation of CB2 limits progression of liver fibrosis by reducing accumulation of liver fibrogenic cells. Activating or increasing CB2's activity provides a method of treating or preventing a liver disease in a subject in need thereof.

In some embodiments, a method of treating or preventing a liver disease in a subject in need thereof, comprises administering to the subject a farnesoid X receptor (FXR) agonist and a cannabinoid receptor 2 agonist.

In some embodiments, the cannabinoid receptor 2 agonist is GW842166X (GlaxoSmithKline). In some embodiments, the cannabinoid receptor 2 agonist is [2-(2,4-dichloroanilino)-N-(tetrahydropyran-4-ylmethyl)-4-(trifluoromethyl)pyrimidine-5-carboxamide]. In some embodiments, an FXR agonist is administered to a subject in need thereof in combination with GW842166X. In some embodiments, an FXR agonist is administered to a subject in need thereof in combination with [2-(2,4-dichloroanilino)-N-(tetrahydropyran-4-ylmethyl)-4-(trifluoromethyl)pyrimidine-5-carboxamide]. In some embodiments, GW842166X is administered at a dose of about 175 mg per day. In some embodiments, a therapeutically effective dose of GW842166X is administered orally.

In some embodiments, the cannabinoid receptor 2 agonist is LY2828360 (Ely Lilly). In some embodiments, an FXR agonist is administered to a subject in need thereof in combination with LY2828360. In some embodiments, an FXR agonist is administered to a subject in need thereof in combination with [2-(2,4-dichloroanilino)-N-(tetrahydropyran-4-ylmethyl)-4-(trifluoromethyl)pyrimidine-5-carboxamide]. In some embodiments, LY2828360 is administered at a dose of about 100 mg per day. In some embodiments, LY2828360 is administered at a dose of about 85 mg per day. In some embodiments, a therapeutically effective dose of LY2828360 is administered orally.

Combination with a Hedgehog (Hh) Antagonist

The Hedgehog pathway plays a critical role for embryonic development and is also involved in adult tissue maintenance, regeneration, and renewal. Activation of the Hh pathway in adults includes proliferation of liver progenitor cells to promote liver regeneration, but also leads to vascular remodeling, inflammation, and liver fibrogenesis. Hh signaling is associated with liver disease such as NAFLD. Blocking, inhibiting, decreasing, or dampening the Hh signaling pathway provides a method of treating or preventing a liver disease in a subject in need thereof.

In some embodiments, a method of treating or preventing a liver disease in a subject in need thereof, comprises administering to the subject a farnesoid X receptor (FXR) agonist and a Hedgehog pathway antagonist. In some embodiments, a method of treating or preventing a liver disease in a subject in need thereof, comprises administering to the subject a farnesoid X receptor (FXR) agonist and a smoothened (SMO) antagonist. In some embodiments, a method of treating or preventing a liver disease in a subject in need thereof, comprises administering to the subject a farnesoid X receptor (FXR) agonist and a Gli-1 antagonist. In some embodiments, a method of treating or preventing a liver disease in a subject in need thereof, comprises administering to the subject a farnesoid X receptor (FXR) agonist and a Gli-2 antagonist.

In some embodiments, the Hedgehog pathway antagonist is vismodegib (Genentech). In some embodiments, an FXR agonist is administered to a subject in need thereof in combination with vismodegib. In some embodiments, vismodegib is administered orally at a dose of about 150 mg per day. In some embodiments, vismodegib is administered orally in the form of a capsule.

In some embodiments, the Hedgehog pathway antagonist is an RNA-mediated interference (RNAi) construct that targets the Hedgehog pathway. In some embodiments, the Hedgehog pathway antagonist is an RNA-mediated interference (RNAi) construct that targets the smoothened (SMO). In some embodiments, the Hedgehog pathway antagonist is an RNA-mediated interference (RNAi) construct that targets the Gli-1 or Gli-2.

In some embodiments, the Hedgehog pathway antagonist is an antisense oligonucleotide that targets the Hedgehog pathway. In some embodiments, the Hedgehog pathway antagonist is an antisense oligonucleotide that targets the smoothened (SMO). In some embodiments, the Hedgehog pathway antagonist is an antisense oligonucleotide that targets the Gli-1 or Gli-2.

Combination with an ASK-1 Inhibitor

Apoptosis signal regulating kinase 1 (ASK-1) is an essential component of the MAP kinase signal transduction pathway. ASK-1 activates downstream c-Jun N-terminal kinase (JNKs) and p38 MAP kinases, which induces production of inflammatory cytokines and cell apoptosis. In liver diseases such as NAFLD, activation of JNKs by ASK-1 induces TGF-beta-mediated apoptosis of hepatocytes. Thus, blocking, inhibiting, decreasing, or dampening ASK-1 provides a method of treating or preventing a liver disease in a subject in need thereof. In some embodiments, a FXR agonist (e.g. Compound 1, or a pharmaceutically acceptable salt thereof), is administered in combination with an ASK-1 inhibitor. In some embodiments, the ASK-1 inhibitor is selonsertib (Gilead), GS444217 (Gilead), or GS459679 (Gilead).

In some embodiments, the ASK-1 antagonist is selonsertib (Gilead; 5-(4-cyclopropyl-1H-imidazol-1-yl)-2-fluoro-N-[6-(4-isopropyl-4H-1,2,4-triazol-3-yl)-2-pyridinyl]-4-methylbenzamide). In some embodiments, selonsertib is administered orally at a dose of 2, 6, or 18 mg once per day.

Combination with a NOX1, NOX2, NOX3, NOX4, NOX5, DUOX1, or DUOX2 Antagonist

NADPH oxidase 1 (NOX1), NADPH oxidase 2 (NOX2), NADPH oxidase 3 (NOX3), NADPH oxidase 4 (NOX4), NADPH oxidase 5 (NOX5), dual oxidase 1 (DUOX1), and dual oxidase (DUOX2) catalyze the production of reactive oxygen species (ROS), which are associated with cell differentiation, signal transduction, and tumor cell growth. ROS produced by NOX1, NOX2, NOX3, NOX4, NOX5, DUOX1, and/or DUOX2 act as signaling molecules alone, act in conjunction with other signaling molecules, or react to other signaling molecules. In addition, ROS produced by NOX1, NOX2, NOX3, NOX4, NOX5, DUOX1, and/or DUOX2 is implicated in fibrosis in general, central nervous system disorders, pain, cardiovascular disease, diabetes, diabetes-induced nephropathy, and metabolic diseases. Furthermore, ROS produced by NOX1, NOX2, NOX3, NOX4, NOX5, DUOX1, and/or DUOX2 also play a key role in liver fibrosis and liver injury. Deficiency of NOX1, NOX2, NOX3, NOX4, NOX5, DUOX1, and/or DUOX2 decreases liver inflammation, fibrosis, and injury. Therefore, blocking, inhibiting, decreasing, or dampening the activity of NOX1, NOX2, NOX3, NOX4, NOX5, DUOX1, DUOX2, or any combination thereof provides a method treating or preventing a liver disease in a subject in need thereof.

In some embodiments, a method of treating or preventing a liver disease in a subject in need thereof, comprises administering to the subject a farnesoid X receptor (FXR) agonist and a NOX1 antagonist. In some embodiments, a method of treating or preventing a liver disease in a subject in need thereof, comprises administering to the subject a farnesoid X receptor (FXR) agonist and a NOX2 antagonist. In some embodiments, a method of treating or preventing a liver disease in a subject in need thereof, comprises administering to the subject a farnesoid X receptor (FXR) agonist and a NOX4 antagonist. In some embodiments, a method of treating or preventing a liver disease in a subject in need thereof, comprises administering to the subject a farnesoid X receptor (FXR) agonist and a NOX1 and NOX4 dual antagonist. In some embodiments, a method of treating or preventing a liver disease in a subject in need thereof, comprises administering to the subject a farnesoid X receptor (FXR) agonist and a NOX2 and NOX4 dual antagonist. In some embodiments, a method of treating or preventing a liver disease in a subject in need thereof, comprises administering to the subject a farnesoid X receptor (FXR) agonist and a NOX1 and NOX2 dual antagonist. In some embodiments, a method of treating or preventing a liver disease in a subject in need thereof, comprises administering to the subject a farnesoid X receptor (FXR) agonist and an antagonist of NOX1, NOX2, NOX3, NOX4, NOX5, DUOX1, DUOX2, or any combination thereof. In some embodiments, a method of treating or preventing a liver disease in a subject in need thereof, comprises administering to the subject a farnesoid X receptor (FXR) agonist and a NOX inhibitor that inhibits any combination of the NOX1-5 and DUOX1-2 isoforms.

In some embodiments, the NOX1 and NOX4 dual antagonist is GKT137831 (Genkyotex; 2-(2-chlorophenyl)-4-[3-(dimethylamino)phenyl]-5-methyl-1H-pyrazolo[4,3-c]pyridine-3,6(2H,5H)-dione).

In some embodiments, the NOX1 and NOX4 dual antagonist is GKT136901 (Genkyotex; 2-(2-chlorophenyl)-4-methyl-5-(pyridin-2-ylmethyl)-1H-pyrazolo-[4,3-c]pyridine-3,6(2H,5H)-dione).

In some embodiments, the NOX1 and NOX4 dual antagonist is GKT771 (Genkyotex).

In some embodiments, the NOX1 antagonist is ML171. In some embodiments, the NOX1 antagonist is 2-acetylphenothiazine

In some embodiments, the NOX inhibitor is VAS2870 (3-benzyl-7-(2-benzoxazolyl)thio-1,2,3-triazolo(4,5-d)pyrimidine).

Combination with a LOXL2 Antagonist

Lysyl oxidase homolog 2 (LOXL2) is an extracellular matrix enzyme that promotes fibrosis via the cross-linkage of collagen and elastin fibers. LOXL2 enhances accumulation and deposition of collagen in certain tissues. LOXL2 is not significantly expressed in normal liver tissues, however, increased expression levels of LOXL2 are found in fibrotic liver diseases. Upregulation of LOXL2 in hepatocytes contributes to liver damage and leads to liver fibrosis. Hence, blocking, inhibiting, decreasing, or dampening LOXL2 provides a method of treating or preventing a liver disease in a subject in need thereof. In some embodiments, a method of treating or preventing a liver disease in a subject in need thereof, comprises administering to the subject a farnesoid X receptor (FXR) agonist and a LOXL2 antagonist.

In some embodiments, the LOXL2 antagonist is an antibody. In some embodiments, an FXR agonist is administered to a subject in need thereof in combination with simtuzumab (Gilead). In some embodiments, simtuzumab is administered at a dose of about 2 mg/kg to about 15 mg/kg of mammal body weight. In some embodiments, simtuzumab is administered subcutaneously at a dose of about 75 mg to 125 mg once per week.

In some embodiments, an FXR agonist is administered to a subject in need thereof in combination with PAT-1251 (Pharmakea). In some embodiments, PAT-1251 is administered at a dose of about 1 mg/kg to about 75 mg/kg of mammal body weight. In some embodiments, PAT-1251 is administered orally at a dose of about 100-2000 mg daily. In some embodiments, PAT-1251 is administered orally at a dose of about 500-1000 mg daily.

Combination with a TGF-beta Antagonist

Transforming growth factor-beta (TGF-beta) is a multifunctional cytokine that plays an important role in tissue repair and wound healing. TGF-beta is found in all tissues and generally, TGF-beta stimulates the production of extracellular matrix proteins as well as inhibits the degradation of these proteins. The balance of these functions is required for maintaining tissue homeostasis. The disruption of TGF-beta's anti-inflammatory and immunosuppressive effects leads to a number of disease processes in the liver. TGF-beta contributes to all disease stages in chronic liver disease, from initial liver injury through inflammation and fibrosis to cirrhosis and hepatocellular carcinoma. TGF-beta is required for liver fibrogenesis to occur; the blunting of TGF-beta signaling reduces liver fibrosis. Thus, blocking, inhibiting, decreasing, or dampening TGF-beta provides a method of treating or preventing a liver disease in a subject in need thereof. In some embodiments, a method of treating or preventing a liver disease in a subject in need thereof, comprises administering to the subject a farnesoid X receptor (FXR) agonist and a TGF-beta antagonist. In some embodiments, a method of treating or preventing a liver disease in a subject in need thereof, comprises administering to the subject a farnesoid X receptor (FXR) agonist and a TGF-beta antagonist.

In some embodiments, the TGF-beta antagonist is pirfenidone. In some embodiments, the TGF-beta antagonist is 5-methyl-1-phenylpyridin-2(1H)-one. In some embodiments, an FXR agonist is administered to a subject in need thereof in combination with pirfenidone. In some embodiments, an FXR agonist is administered to a subject in need thereof in combination with 5-methyl-1-phenylpyridin-2(1H)-one. In some embodiments, pirfenidone is administered orally at a dose of about 250 mg to about 2500 mg per day. In some embodiments, pirfenidone is administered orally in the form of a capsule. In some embodiments, pirfenidone is administered orally with food at a dose of about 267 mg per capsule, three capsules per day, during the first week of treatment. In some embodiments, pirfenidone is administered orally with food at a dose of about 267 mg per capsule, two capsules three times per day to give a total of about 1602 mg per day, during the second week of treatment. In some embodiments, pirfenidone is administered orally with food at a dose of about 267 mg per capsule, three capsules three times per day to give a total of 2403 mg per day, after the first 15 day of treatment.

Combinations with Metabolic Therapeutic Agents

In some embodiments, a method of treating or preventing a liver disease in a subject in need thereof, comprises administering to the subject a farnesoid X receptor (FXR) agonist and an additional metabolic therapeutic agent. In some embodiments, a method of treating or preventing a fibrotic liver disease in a subject in need thereof, comprises administering to the subject a farnesoid X receptor (FXR) agonist and an additional metabolic therapeutic agent. In some embodiments, a method of treating or preventing a metabolic liver disease in a subject in need thereof, comprises administering to the subject a farnesoid X receptor (FXR) agonist and an additional metabolic therapeutic agent.

Combination with a PPAR Delta Agonist

Peroxisome proliferator-activated receptor delta (PPAR delta) is a nuclear hormone receptor that is involved in a variety of chronic diseases such as diabetes, obesity, atherosclerosis, and cancer. Specifically, PPAR delta is an important regulator of fatty acid metabolic pathways, glucose metabolism, and adipocyte proliferation, differentiation, and apoptosis. PPAR delta agonists modulate glucose metabolism, fatty acid metabolism, and alleviate insulin resistance. PPAR delta agonists inhibit formation of lipid deposits within hepatocytes and inhibit the development of liver steatosis. Therefore, activating or increasing PPAR delta provides a method of treating or preventing a liver disease in a subject in need thereof. In some embodiments, a method of treating or preventing a liver disease in a subject in need thereof, comprises administering to the subject a farnesoid X receptor (FXR) agonist and a PPAR delta agonist. In some embodiments, a method of treating or preventing a liver disease in a subject in need thereof, comprises administering to the subject a farnesoid X receptor (FXR) agonist and a PPAR delta agonist.

In some embodiments, the PPAR delta agonist is KD-3010 (Kalypsys). In some embodiments, an FXR agonist is administered to a subject in need thereof in combination with KD-3010. In some embodiments, KD-3010 is administered orally at a dose of about 5 mg to about 200 mg per day. In some embodiments, KD-3010 is administered orally in the form of a capsule. In some embodiments, KD-3010 is administered orally at a dose of about 10 mg once per day, about 20 mg once per day, about 30 mg once per day, about 40 mg once per day, about 60 mg once per day, or about 80 mg once per day.

In some embodiments, the PPAR delta agonist is KD-3020 (Kalypsys).

Combinations with a PPAR Alpha Agonist or a PPAR Delta/PPAR Alpha Agonist

PPAR alpha, also known as NR1C1 (nuclear receptor 1, group C, member 1), is a major regulator of lipid metabolism in the liver. PPAR alpha is activated during energy deprivation conditions and once activated, PPAR alpha promotes uptake and catabolism of fatty acids. PPAR alpha expression is reduced with high fat intake. PPAR alpha agonists decrease hepatic steatosis by increasing mitochondrial-beta oxidation and reducing lipogenesis. Administration of PPAR alpha agonists also result in body mass loss. Therefore, activating or increasing PPAR alpha provides a method of treating or preventing a liver disease in a subject in need thereof. In some embodiments, a method of treating a liver disease in a subject in need thereof, comprises administering to the subject a farnesoid X receptor (FXR) agonist and a PPAR alpha agonist. In some embodiments, a method of treating a liver disease in a subject in need thereof, comprises administering to the subject a farnesoid X receptor (FXR) agonist and a PPAR delta agonist. In some embodiments, a method of treating a liver disease in a subject in need thereof, comprises administering to the subject a farnesoid X receptor (FXR) agonist and a dual PPAR delta/PPAR alpha agonist.

In some embodiments, the PPAR alpha agonist is a fibrate. In some embodiments, the PPAR alpha agonist is fenofibrate. In some embodiments, an FXR agonist is administered to a subject in need thereof in combination with a fibrate. In some embodiments, an FXR agonist is administered to a subject in need thereof in combination with fenofibrate. In some embodiments, fenofibrate is administered orally at a dose of about 40 mg to about 200 mg per day. In some embodiments, fenofibrate is administered orally in the form of a capsule. In some embodiments, fenofibrate is administered orally at a dose of about 150 mg once per day. In some embodiments, fenofibrate is administered orally at a dose of about 120 mg once per day.

In some embodiments, the PPAR alpha agonist is a nutriceutical. In some embodiments, the PPAR alpha agonist is fish oil. In some embodiments, an FXR agonist is administered to a subject in need thereof in combination with fish oil. In some embodiments, fish oil comprises alpha-linoleic acid, eicosapentaenoic acid (EPA), and docosahexaenoic acid (DHA). In some embodiments, fish oil is administered orally at a dose of about 100 mg to about 5,000 mg per day. In some embodiments, fish oil is administered orally in the form of a capsule. In some embodiments, fish oil is administered orally at a dose of about 2,000 mg once per day. In some embodiments, fish oil is administered orally at a dose of about 4,000 mg once per day.

In some embodiments, the PPAR delta/PPAR alpha dual agonist is elafibranor (Genfit). In some embodiments, an FXR agonist is administered to a subject in need thereof in combination with elafibranor. In some embodiments, elafibranor is administered orally at a dose of about 70 mg to about 130 mg per day. In some embodiments, elafibranor is administered orally in the form of a capsule. In some embodiments, elafibranor is administered orally at a dose of about 80 mg once per day, or about 120 mg once per day.

Combinations with an Inhibitor of Acetyl-CoA Carboxylase (ACC)

Acetyl-CoA carboxylase (ACC) is a biotin-dependent enzyme that catalyzes the irreversible carboxylation of acetyl-CoA to produce malonyl-CoA. ACC catalyzes the rate-limiting step in de novo lipogenesis (DNL). Increased DNL contributes to the pathogenesis of NASH. ACC inhibition improves steatosis, liver inflammation, and liver fibrosis.

In some embodiments, a FXR agonist (e.g. Compound 1, or a pharmaceutically acceptable salt thereof), is administered in combination with an ACC inhibitor. In some embodiments, the ACC inhibitor is GS-0976. In some embodiments, GS-0976 is administered orally at a dose of about 5 to 20 mg once per day. In some embodiments, GS-0976 is administered orally at a dose of 5 mg once per day. In some embodiments, GS-0976 is administered orally at a dose of 20 mg once per day.

Combinations with a GLP1 Agonist

Insulin resistance (IR) in both liver and adipose tissue is believed to be a key driver in the pathogenesis of NASH. Subjects with NASH have severe adipose IR, alongside increased hepatic IR, and de novo lipogenesis (DNL). Collectively these contribute to excess lipid accumulation in the liver and the overspill of non-esterified fatty acids (NEFA) and release of triglyceride-derived toxic metabolites from adipose tissue lipolysis, form the primary lipotoxic insult in the pathogenesis of NASH. In addition to driving intrinsic hepatic IR and inflammation, hepatic lipotoxicity is thought to further fuel the circulating pro-inflammatory environment and IR status in NASH, which in turn contributes to the cycle of worsening adipose dysfunction and lipolysis.

Glucagon-like peptide-1 (GLP-1) agonists have been shown to improve glycemic control, contribute to weight loss, improve insulin sensitivity, improve liver enzymes and reduce hepatic glucose production. Improvements in hepatic steatosis following GLP-1 therapy has been observed, which in some cases was accompanied by reductions in oxidative stress and fibrosis.

In some embodiments, a FXR agonist (e.g. Compound 1, or a pharmaceutically acceptable salt thereof), is administered in combination with a GLP1 agonist. In some embodiments, the GLP1 agonist is liraglutide (Novo), Semaglutide, exenatide (AstraZeneca), dulaglutide (Eli Lilly), lixisenatide (Sanofi), or albiglutide (GSK).

Combination with a DGAT Inhibitor

NASH is characterized by excessive triglycerides (TG) in the liver with concurrent inflammation and cellular damage. Diacylglycerol acyltransferase (DGAT) catalyzes the final step in TG synthesis from diacylglycerol and Acyl-CoA. The reaction catalyzed by DGAT is considered the terminal and only committed step in triglyceride synthesis and to be essential for intestinal absorption (i.e. DGAT1) and adipose tissue formation (i.e. DGAT2). There are two isoforms, DGAT1 and DGAT2, with distinct protein sequences and potentially different physiological functions.

Dietary triglycerides cannot be absorbed directly in the gastrointestinal tract and are broken down into free fatty acids and monoglycerol in the intestine by pancreatic lipase. Once absorbed, the free fatty acids and glycerol are reassembled into triglycerides at the site of absorption, called an enterocyte, and packaged into chylomicron particles to be transported in the lymphatic system to be used throughout the body. DGAT-1 is one of two enzymes that catalyze the steps of triglyceride biosynthesis from mono- or diacylglycerol and fatty acids, and is mainly distributed in the intestine, liver and adipose tissue

Inhibition of the enzyme has shown to reduce fat storage in animal models and clinical trials, leading to reduction of body weight.

In some embodiments, a FXR agonist (e.g. Compound 1, or a pharmaceutically acceptable salt thereof), is administered in combination with a DGAT1 inhibitor or DGAT2 inhibitor. In some embodiments, a FXR agonist (e.g. Compound 1, or a pharmaceutically acceptable salt thereof), is administered in combination with a DGAT1 inhibitor. In some embodiments, the DGAT1 inhibitor is GSK3008356. In some embodiments, a FXR agonist (e.g. Compound 1, or a pharmaceutically acceptable salt thereof), is administered in combination with a DGAT2 inhibitor. In some embodiments, the DGAT2 inhibitor is PF-0685571
Combination with a Bile Acid Pathway Modulator

Bile acids bind to receptors in the colon that promote the release of intestinal hormones, such as glucagon-like peptide1 (GLP1). In the liver, bile acids bind to other receptors that regulate bile acid production from cholesterol in a negative feedback loop. Under normal conditions, bile acids bind to these receptors and inhibit the synthesis of new bile acids. As bile acid levels are lowered, the liver must produce needed bile acids from cholesterol, which requires increased uptake of cholesterol and a resulting decrease of cholesterol in the liver. A decrease of cholesterol accumulation in the liver reduces liver damage in liver diseases such as, but not limited to NASH and NAFLD.

After completing digestion, bile acids are reclaimed in the distal part of the small intestine, known as the terminal ileum, by ileal bile acid transporters (IBAT; also called ASBT or apical sodium-bile acid transporter). IBAT initiates the transport of bile acids, which flow through the portal vein, back to the liver in a process known as enterohepatic circulation.

In some embodiments, a FXR agonist (e.g. Compound 1, or a pharmaceutically acceptable salt thereof), is administered in combination with an IBAT inhibitor. In some embodiments, the IBAT inhibitor is volixibat (also known as SHP626), maralixibat (Shire), elobixibat (Albireo), or A4350 (Albireo). In some embodiments, the IBAT inhibitor is volixibat.

Combination with a Fibroblast Growth Factor Receptor Modulator

In some embodiments, a FXR agonist (e.g. Compound 1, or a pharmaceutically acceptable salt thereof), is administered in combination with a modulator of the fibroblast growth factor (FGF) 19 receptor or fibroblast growth factor (FGF) 21 receptor. In some embodiments, a FXR agonist (e.g. Compound 1, or a pharmaceutically acceptable salt thereof), is administered in combination with a FGF19 variant, or a FGF21 variant.

The human hormone FGF19 is a primary regulator of bile acid synthesis in the liver and a key signaling molecule in metabolic processes involved in body weight maintenance, including glucose homeostasis and triglyceride regulation. FGF19 binds to the FGF19 receptor resulting in reduction of liver fat content, improvement in liver steatosis, inflammation and fibrosis and improves liver function by targeting multiple pathogenic pathways of nonalcoholic steatohepatitis (NASH).

In some embodiments, a FXR agonist (e.g. Compound 1, or a pharmaceutically acceptable salt thereof), is administered in combination with a variant of human FGF19. In some embodiments, the variant of human FGF19 is an engineered variant of the human hormone FGF19. In some embodiments, the variant of human FGF19 is NGM282 (NGM/Merck).

Fibroblast growth factor 21 (FGF21) is a key regulator of metabolism expressed in numerous tissues, including the liver. Many different metabolically active tissues express FGF21, but most of the hormone is produced by the liver. Levels of FGF21 are regulated by metabolic stressors such as obesity, lack of physical exercise and metabolic diseases such as type 2 diabetes. Conditions where elevated circulating FGF21 levels are found include obesity, type 2 diabetes, cardiovascular disease, non-alcoholic fatty liver disease (NAFLD), and non-alcoholic steatohepatitis (NASH). These elevations may represent a compensatory response to protect the body from adverse metabolic conditions.

In some embodiments, a FXR agonist (e.g. Compound 1, or a pharmaceutically acceptable salt thereof), is administered in combination with a variant of human FGF21. In some embodiments, a FXR agonist (e.g. Compound 1, or a pharmaceutically acceptable salt thereof), is administered in combination with a PEGylated fibroblast growth factor (FGF) 21. In some embodiments, the PEGylated fibroblast growth factor (FGF) 21 is BMS-986036 (Bristol-Myers-Squibb).

Combination with a Stearoyl-CoA desaturase (SCD1) Inhibitor and/or ATP-Binding Cassette Transporter A1 Modulator

In some embodiments, a FXR agonist (e.g. Compound 1, or a pharmaceutically acceptable salt thereof), is administered in combination with a stearoyl-CoA desaturase (SCD1) inhibitor or an adenosine triphosphate binding cassette transporter A1 (ABCA1) modulator.

Stearoyl-CoA desaturase (SCD1) catalyzes the rate-limiting step in the biosynthesis of monounsaturated fatty acids and is considered a potent mechanism in liver steatosis and fibrosis. Mice with a targeted disruption of the SCD1 isoform have reduced body adiposity, increased energy expenditure, and up-regulated expression of several genes encoding enzymes and transcription factors enhancing fatty acid oxidation and reducing fibrogenesis in liver including AMPK and SIRT. SCD1 inhibition decreases the synthesis and increases b oxidation of fatty acids, resulting in decreased hepatic storage of triglycerides and fatty acid esters. This process reduces liver fat and improves insulin resistance in animals.

In some embodiments, a FXR agonist (e.g. Compound 1, or a pharmaceutically acceptable salt thereof), is administered in combination with a stearoyl-CoA desaturase (SCD1) inhibitor. In some embodiments, the SCD1 inhibitor is a liver targeted SCD 1 inhibitor. In some embodiments, the SCD1 inhibitor is arachidyl amido cholanoic acid (Aramchol), a fatty acid bile acid conjugate. Aramchol is a liver targeted SCD 1 modulator with a dual mode of action on liver fibrosis, down regulation of steatosis and a direct effect on hepatic Stellate Cells (HSCs), the human collagen producing cells. Aramchol has shown down regulation of liver steatosis and fibrosis in multiple animal models demonstrating its effect on the three key pathologies of NASH: steatosis, inflammation and fibrosis.

The adenosine triphosphate-binding cassette transporter A1 (ABCA1) is a pan-cellular cholesterol export pump, and stimulating the activity of this transporter in animal studies has shown an anti-atherogenic effect. A decrease in ABCA1 protein expression and function may contribute to an increase in lipid storage in hepatocytes, a detrimental effect for liver diseases, such as NASH and NAFLD.

In some embodiments, a FXR agonist (e.g. Compound 1, or a pharmaceutically acceptable salt thereof), is administered in combination with an adenosine triphosphate binding cassette transporter A1 (ABCA1) modulator. In some embodiments, the ABCA1 modulator is arachidyl amido cholanoic acid. Arachidyl amido cholanoic acid activates cholesterol efflux by stimulating the adenosine triphosphate—binding cassette transporter A1, a pan-cellular cholesterol export pump.

Combination with a Thyroid Hormone Beta Agonist

Thyroid hormone regulation of lipid metabolism affects a wide range of interrelated health parameters, from levels of cholesterol and triglycerides in the blood to the pathological buildup of fat in the liver. In some embodiments, selective thyroid hormone receptor beta (THR-β) activation in the liver improves dysregulation of lipid metabolism, results in a reduction of liver fat, lowering of multiple atherogenic lipids including LDL-cholesterol and triglycerides, and resolution of NASH.

In some embodiments, a FXR agonist (e.g. Compound 1, or a pharmaceutically acceptable salt thereof), is administered in combination with a thyroid hormone beta agonist. In some embodiments, the thyroid hormone beta agonist is MGL-3196 (Madrigal Pharmaceuticals), MGL-3745 (Madrigal Pharmaceuticals) or VK2809 (Viking Therapeutics).

### Other Combinations

In some embodiments, a FXR agonist (e.g. Compound 1, or a pharmaceutically acceptable salt thereof), is administered in combination with a glucose lowering agent, an insulin secretion stimulator, an insulin sensitizer, a lipid lowering agent, a compound that increases sympathetic nervous system activity, ethyl eicosapentaenoate, obeticholic acid, or a TGR5 agonist.

In some embodiments, a FXR agonist (e.g. Compound 1, or a pharmaceutically acceptable salt thereof), is administered in combination with a statin, an insulin sensitizing drug, an insulin secretagogue, an alpha-glucosidase inhibitor, a GLP agonist, a DPP-4 inhibitor (such as sitagliptin, vildagliptin, saxagliptin, linagliptin, anagliptin, teneligliptin, alogliptin, gemigliptin, or dutogliptin), a catecholamine (such as epinephrine, norepinephrine, or dopamine), peroxisome proliferator-activated receptor (PPAR)-gamma agonist (e.g., a thiazolidinedione (TZD) [such as ioglitazone, rosiglitazone, rivoglitazone, or troglitazone], aleglitazar, farglitazar, muraglitazar, or tesaglitazar), or a combination thereof. In some cases, the statin is a HMG-CoA reductase inhibitor. In other instances, additional therapeutic agents include fish oil, fibrate, vitamins such as niacin, retinoic acid (e.g., 9 cis-retinoic acid), nicotinamide ribonucleoside or its analogs thereof, or combinations thereof. In some instances, nicotinamide ribonucleoside or its analogs thereof, which promote NAD⁺ production, a substrate for many enzymatic reactions including p450s which is a target for FXR (e.g., see Yang et al., J. Med. Chem. 50:6458-61, 2007).

In some embodiments, a FXR agonist is administered in combination with an additional therapeutic agent such as a statin, an insulin sensitizing drug, an insulin secretagogue, an alpha-glucosidase inhibitor, a GLP agonist, a DPP-4 inhibitor (such as sitagliptin, vildagliptin, saxagliptin, linagliptin, anagliptin, teneligliptin, alogliptin, gemigliptin, or dutogliptin), a catecholamine (such as epinephrine, norepinephrine, or dopamine), peroxisome proliferator-activated receptor (PPAR)-gamma agonist (e.g., a thiazolidinedione (TZD) [such as ioglitazone, rosiglitazone, rivoglitazone, or troglitazone], aleglitazar, farglitazar, muraglitazar, or tesaglitazar), or combinations thereof, for the treatment of diabetes or diabetes related disorder or conditions. In some embodiments, a FXR agonist is administered in combination with an additional therapeutic agent such as fish oil, fibrate, vitamins such as niacin, retinoic acid (e.g., 9 cis-retinoic acid), nicotinamide ribonucleoside or its analogs thereof, or combinations thereof, for the treatment of diabetes or diabetes related disorder or conditions.

In some embodiments, a FXR agonist is administered in combination with a statin such as a HMG-CoA reductase inhibitor, fish oil, fibrate, niacin, or a combination thereof, for the treatment of dyslipidemia.

In additional embodiments, a FXR agonist is administered in combination with a vitamin such as retinoic acid for the treatment of diabetes and diabetes related disorder or condition such as lowering elevated body weight and/or lowering elevated blood glucose from food intake.

In some embodiments, the farnesoid X receptor agonist is administered with at least one additional therapy. In some embodiments, the at least one additional therapy is a glucose-lowering agent. In some embodiments, the at least one additional therapy is an anti-obesity agent. In some embodiments, the at least one additional therapy is selected from among a peroxisome proliferator activated receptor (PPAR) agonist (gamma, dual, or pan), a dipeptidyl peptidase (IV) inhibitor, a glucagon-like peptide-1 (GLP-I) analog, insulin or an insulin analog, an insulin secretagogue, a sodium glucose co-transporter 2 (SGLT2) inhibitor, a glucophage, a human amylin analog, a biguanide, an alpha-glucosidase inhibitor, a meglitinide, a thiazolidinedione, and sulfonylurea. In some embodiments, the at least one additional therapy is metformin, sitagliptin, saxagliptin, repaglinide, nateglinide, exenatide, liraglutide, insulin lispro, insulin aspart, insulin glargine, insulin detemir, insulin isophane, and glucagon-like peptide 1, or any combination thereof. In some embodiments, the at least one additional therapy is a lipid-lowering agent. In certain embodiments, the at least one additional therapy is administered at the same time as the farnesoid X receptor agonist. In certain embodiments, the at least one additional therapy is administered less frequently than the farnesoid X receptor agonist. In certain embodiments, the at least one additional therapy is administered more frequently than the farnesoid X receptor agonist. In certain embodiments, the at least one additional therapy is administered prior to administration of the farnesoid X receptor agonist. In certain embodiments, the at least one additional therapy is administered after administration of the farnesoid X receptor agonist.

Combination with Bariatric Surgery

Current best treatments of NAFLD and NASH include weight reduction with the current options being life style modifications, with or without pharmaceuticals, and bariatric surgery. Bariatric surgery is an effective treatment option for individuals who are severely obese (body mass index ≤35 kg/m2), and provides for long-term weight loss and resolution of obesity-associated diseases in most patients. Regression and/or histologic improvement of NASH have been documented after bariatric surgery.

In some embodiments, a FXR agonist (e.g. Compound 1, or a pharmaceutically acceptable salt thereof), is administered in combination with bariatric surgery.

Bariatric surgery techniques can be done using the laparoscopic approach. One technique is adjustable gastric banding (AGB), in which an inflatable and adjustable silicone band is placed around the upper stomach, close to the gastroesophageal junction, to create a 30-mL proximal gastric pouch. After surgery, a series of stepwise adjustments to constrict the band stoma are made in the outpatient office.

Another technique in bariatric surgery is Roux-en-Y gastric bypass (RYGB).This is a proximal gastric bypass. A small 30- to 50-mL proximal gastric pouch is created by dividing it from the larger stomach using staplers. The gastric pouch is then connected to the proximal jejunum in a Roux-en-Y fashion, using a variety of equally effective laparoscopic anastomotic techniques.

Another technique is sleeve gastrectomy (SG) in which a left lateral portion of the gastric antrum, body, and fundus is separated from the medial portion. The “larger excess stomach” is removed from the abdominal cavity, leaving the smaller, left curvature-based, narrow stomach, preserving the pylorus and usual connection with the duodenum.

Still another technique is biliopancreatic diversion without (BPD) or with duodenal-switch (BPD-DS). With this technique, a partial gastrectomy (BPD) or sleeve gastrectomy (BPD-DS) is created, and the small bowel is divided in two sections of similar length (alimentary and biliopancreatic limb).The alimentary limb is connected to the first portion of the duodenum (BPD-DS) or the stomach (BPD). The biliopancreatic limb is anastomosed to the distal small intestine.

Still another technique is vertical banded gastroplasty (VBG) that combines stomach stapling and gastric banding, which is not adjustable, to create a small gastric pouch. After an incision into the stomach is made, the sides of the incision are stapled, creating a hole in the stomach for the band to loop through. Above the created hole, the stomach is stapled.

In some embodiments, a FXR agonist (e.g. Compound 1, or a pharmaceutically acceptable salt thereof), is administered in combination with bariatric surgery. In some embodiments, the bariatric surgery techniques is gastric banding, gastric bypass, sleeve gastrectomy, biliopancreatic diversion without or with duodenal-switch, or vertical banded gastroplasty. In some embodiments, the bariatric surgery techniques is adjustable gastric banding (AGB), Roux-en-Y gastric bypass (RYGB), sleeve gastrectomy (SG), biliopancreatic diversion without (BPD) or with duodenal-switch (BPD-DS), or vertical banded gastroplasty (VBG).

In some embodiments, bariatric surgery is restrictive surgery, malabsorptive surgery, or a combination of both restrictive and malabsorptive surgery. In some embodiments, restrictive bariatric surgeries include, but are not limited to vertical banded gastroplasty, adjustable gastric band, sleeve gastrectomy, intragastric balloon (gastric balloon), or gastric plication. In some embodiments, malabsorptive bariatric surgeries include, but are not limited to biliopancreatic diversion, jejunoileal bypass, or endoluminal sleeve. In some embodiments, the combination of both malabsorptive and restrictive bariatric surgeries include, but are not limited to gastric bypass surgery, sleeve gastrectomy with duodenal switch, or implantable gastric stimulation.

Combination with Vitamins

In some embodiments, a FXR agonist (e.g. Compound 1, or a pharmaceutically acceptable salt thereof), is administered in combination with a vitamin. In some embodiments, the vitamin is administered parenterally or enterally. In some embodiments, the vitamin is tocopherol, alpha-tocopherol, vitamin E, gamma-tocopherol, tocotrienol, beta-tocopherol, or delta-tocopherol.

Combination with Bacteria

Microbial products have been shown to contribute to the development or maintenance of liver steatosis and inflammation, which contribute significantly to the development of NASH and NAFLD. The microbiome is influenced by a number of factors that contribute to the development of inflammation and liver steatosis. Gut microbiota are thought to play a role in the pathogenesis of NASH for several reasons. First, gut microbiota are known to have a large effect on the digestion and absorption of nutrients. Second, gut microbiota participate in the development and homeostasis of the overall immunity of the host. Therefore, certain microbiota influence the development of liver inflammation. The links between gut microbiota and the host immune system include, but are not limited to toll-like receptors (TLRs) and short-chain fatty acids. In some embodiments, the innate immune system influences the metabolic syndrome and obesity. Third, gut microbiota influence the production of gut hormones, such as glucagon-like peptide 1, and, subsequently, have an effect on the overall metabolism of the host. The liver appears as the first point of contact for (and produces the initial immunological response to) bacteria and microbial components, as well as other endogenous and exogenous toxins present in the portal blood. Given the capacity of the liver to regulate metabolism in a form that affects the entire organism, to distribute numerous substances to the gut through bile and the entero-hepatic circulation, and to regulate numerous hormonal and immunological responses, the potential for the liver to influence gut function can be quickly appreciated. Interactions between the gut, the diet and the liver are, naturally, bidirectional; hormones, inflammatory mediators, and the products of digestion and absorption all unequivocally influence liver function.

In some embodiments, the microbiome is influenced by a number of factors that contribute to the development of inflammation and liver steatosis; non-limiting examples of these factors include short-chain fatty acids (SCFAs) and lipopolysaccharide (LPS).

Altered gut microbiota causes obesity; this relationship is attributed to short-chain fatty acids (SCFAs). The amount of SCFAs in the gut of obese subjects is elevated compared to the SCFAs levels in the gut of healthy subjects. Obese subjects have increased levels of gut bacteria that have a greater capacity to harvest energy (e.g. Bacteroidetes/Firmicutes ratio); in other words, these bacteria have are able to produce higher amounts of SCFAs. Altered gut microbiota and fatty liver diseases have recently been associated. It has been shown SFCAs affect the liver through different mechanisms: altered gut microbiota lead to greater calorie intake and elevated SCFAs enhance nutritional intestinal absorption. Both mechanisms contribute to the development of obesity, which is linked to liver disease. Increased production of alcohol by gut microbiota is another mechanism by which altered gut microbiota affects the liver. For example, pediatric NASH patients exhibited elevated serum alcohol concentration than those of healthy controls and non-NASH obese patients. Alcohol produced by gut microbes contributes to the development of NASH by mechanisms similar to those of alcoholic steatohepatitis.

Yet another mechanism by which altered gut microbiome is associated with NAFLD and NASH is through elevated microbial cell components such as lipopolysaccharide (LPS) (i.e. endotoxins), which is found in gram negative bacteria. NASH patients have increased levels of gram negative bacteria in their gut microbiota. NAFLD and NASH patients also exhibit elevated serum endotoxin levels. In addition, in vivo murine studies have shown elevated serum LPS levels lead to metabolic syndrome.

In some embodiments, the additional therapeutic agent administered in combination with an FXR agonist described herein is a probiotic. In some embodiments, the probiotic has an anti-fibrotic, metabolic, or anti-inflammatory effect. In some embodiments, the probiotic alters the metabolism of lipids. In some embodiments, a method of treating or preventing a liver disease in a subject in need thereof, comprises administering to the subject a farnesoid X receptor (FXR) agonist and a probiotic. In some embodiments, the probiotic is a microbe, a spore, a virus, a phage, or any combinations thereof. In some embodiments, the probiotic comprises Streptococcus, Bifidobacterium, Lactobacillus, or any combinations thereof. In some embodiments, the probiotic decreases alcohol production in a subject. In some embodiments, the probiotic decreases alcohol dehydrogenase activity. In some embodiments, the probiotic decreases production of LPS. In some embodiments, the probiotic decreases the presence of gram negative bacteria in the gut. In some embodiments, the probiotic regulates production of SCFAs. In some embodiments, the probiotic decreases production of SCFAs.

### Combinations Suitable for Gastrointestinal Diseases or Conditions

In some embodiments, a FXR agonist (e.g. Compound 1, or a pharmaceutically acceptable salt thereof), is administered in combination with anti-inflammatory agents, monoclonal antibodies, or combinations thereof.

In some embodiments, a FXR agonist (e.g. Compound 1, or a pharmaceutically acceptable salt thereof), is administered in combination with 5-aminosalicylate acid agent, a corticosteroid, an immunomodulator, a TNF alpha inhibitor, an integrin inhibitor, endothelial adhesion molecule (MAdCAM) inhibitor, a JAK kinase inhibitor, an IL-12/23 inhibitor, or a S1P1 selective agonist.

5-Aminosalicylate acid agents include, but are not limited to, sulfasalazine, mesalamine, olsalazine.

Corticosteroids include, but are not limited to, prednisone, budesonide, prednisolone, methylprednisolone.

Immunomodulators include, but are not limited to, azathioprine, 6-mercaptopurine, cyclosporine.

TNF alpha inhibitors include, but are not limited to, adalimumab, infliximab, golimumab.

Integrin inhibitors include, but are not limited to, natalizumab, vedolizumab, etrolizumab

Endothelial adhesion molecules (MAdCAM) inhibitors include, but are not limited to, PF-00547659.

JAK kinase inhibitors include, but are not limited to, tofacitinib, baricitinib, filgotinib, and upadacitinib.

IL-12/23 inhibitors include, but are not limited to, ustekinumab

S1P1 selective agonists include, but are not limited to, ozanimod, etrasimod

In some instances, a FXR agonist is administered in combination with an additional therapeutic agent such as an antibiotic, a corticosteroid, or an additional anti-inflammatory or immuno-modulatory therapy, for the treatment of inflammation related intestinal conditions. In some cases, a FXR agonist is administered in combination with metronidazole, vancomycin, fidaxomicin, corticosteroid, or combinations thereof, for the treatment of inflammation related intestinal conditions. In some embodiments, an FXR agonist is administered in combination with pentoxifylline, an anti-inflammatory and vasodilator medication.

Inflammation is sometimes associated with pseudomembranous colitis. In some instances, pseudomembranous colitis is associated with bacterial overgrowth (such as C. dificile overgrowth). In some embodiments, a FXR agonist is administered in combination with an antibiotic such as metronidazole, vancomycin, fidaxomicin, or a combination thereof, for the treatment of inflammation associated with bacterial overgrowth (e.g., pseudomembranous colitis). In some embodiments, an FXR agonist is administered in combination with solithromycin, a ketolide antibiotic (Cempra).

In some embodiments, a FXR agonist (e.g. Compound 1, or a pharmaceutically acceptable salt thereof), is administered in combination with an opioid agonist, bile acid sequestrant, anticholinergic, tricyclic antidepressant, 5-HT3 antagonist, mixed opioid receptor agonist/antagonist, antimicrobial, neurokinin antagonist, or combination thereof.

In some embodiments, the opioid agonist is loperamide.

In some embodiments, the bile acid sequestrant is cholestyramine, colestipol, or colesevelam.

In some embodiments, the anticholinergic is dicyclomine.

In some embodiments, the tricyclic antidepressant is imipramine, desipramine, or nortriptyline.

In some embodiments, the 5-HT3 antagonist is alosetron, or ramosetron.

In some embodiments, the mixed opioid receptor agonist/antagonist is eluxadoline, or ORP-101.

In some embodiments, the antimicrobial is rifaximin.

In some embodiments, the neurokinin antagonist is ibodutant.

### Pharmaceutical Compositions

Described herein, in certain embodiments, are pharmaceutical compositions comprising a farnesoid X receptor (FXR) agonist and an additional therapeutic agent. Described herein, in certain embodiments, are pharmaceutical compositions comprising a farnesoid X receptor (FXR) agonist and an additional anti-fibrotic therapeutic agent. Disclosed herein in certain embodiments, are pharmaceutical compositions comprising an FXR agonist and an additional metabolic therapeutic agent. In some embodiments, the additional therapeutic agent is any therapeutic agent described herein.

Pharmaceutical compositions are formulated in a conventional manner using one or more pharmaceutically acceptable inactive ingredients that facilitate processing of the active compounds into preparations that is used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

In some embodiments, the pharmaceutical compositions described herein are administered parenterally, enterally, or topically. In some embodiments, the components of the pharmaceutical compositions described herein are administered either alone or in combination with pharmaceutically acceptable carriers, excipients or diluents, in a pharmaceutical composition. Administration of the components and compositions described herein is effected by any method that enables delivery of the compounds to the site of action. These methods include, though are not limited to delivery via enteral routes (including oral, gastric or duodenal feeding tube, rectal suppository and rectal enema), parenteral routes (injection or infusion, including intra-arterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route depends upon for example the condition and disorder of the recipient. By way of example only, the pharmaceutical compositions described herein are administered locally to the area in need of treatment, by for example, local infusion during surgery, topical application such as creams or ointments, injection, catheter, or implant. In some embodiments, the administration is by direct injection at the site of a diseased tissue or organ. In some embodiments, the pharmaceutical compositions described herein are administered orally.

In some embodiments, the pharmaceutical compositions described herein are in a form of a powder, a granule, a micro-pellet, a tablet, a capsule, a suspension, a liquid, a nanoparticle, a microparticle, a liposome, a gel, a dispersion, a solution, an emulsion, an ointment, or a lotion.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which used orally include tablets, push-fit capsules made of gelatin, as well as soft, sealed capsules made of gelatin and a plasticizer, such as glycerol or sorbitol. Tablets are made by compression or molding, optionally with one or more accessory ingredients. Compressed tablets are prepared by compressing in a suitable machine the active ingredient in a free-flowing form such as a powder or granules, optionally mixed with binders, inert diluents, or lubricating, surface active or dispersing agents. Molded tablets are made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions is used, which optionally contain gum arabic, talc, polyvinyl pyrrolidone, carbopol gel, polyethylene glycol, and/or titanium dioxide, lacquer solutions, and suitable organic solvents or solvent mixtures. In some embodiments, dyestuffs or pigments are added to the tablets or Dragee coatings for identification or to characterize different combinations of active compound doses.

In some embodiments, pharmaceutical compositions are formulated for parenteral administration by injection, e.g., by bolus injection or continuous infusion. In some embodiments, formulations for injection are presented in unit dosage form, e.g., in ampoules or in multi-dose containers, with an added preservative. In some embodiments, the compositions take such forms as suspensions, solutions or emulsions in oily or aqueous vehicles, and contain formulatory agents such as suspending, stabilizing and/or dispersing agents. In some embodiments, the compositions are presented in unit-dose or multi-dose containers, for example sealed ampoules and vials, and are stored in powder form or in a freeze-dried (lyophilized) condition requiring only the addition of the sterile liquid carrier, for example, saline or sterile pyrogen-free water, immediately prior to use. In some embodiments, extemporaneous injection solutions and suspensions are prepared from sterile powders, granules and tablets of the kind previously described.

In some embodiments, pharmaceutical compositions for parenteral administration include aqueous and non-aqueous (oily) sterile injection solutions of the active compounds contain antioxidants, buffers, bacteriostats and solutes which render the formulation isotonic with the blood of the intended recipient; and aqueous and non-aqueous sterile suspensions which include suspending agents and thickening agents. Suitable lipophilic solvents or vehicles include fatty oils such as sesame oil, or synthetic fatty acid esters, such as ethyl oleate or triglycerides, or liposomes. In some embodiments, aqueous injection suspensions contain substances which increase the viscosity of the suspension, such as sodium carboxymethyl cellulose, sorbitol, or dextran. In some embodiments, optionally, the suspension contains suitable stabilizers or agents which increase the solubility of the compounds to allow for the preparation of highly concentrated solutions.

In some embodiments, pharmaceutical compositions are also formulated as a depot preparation. In some embodiments, such long acting formulations are administered by implantation (for example subcutaneously or intramuscularly) or by intramuscular injection. Thus, for example, the compounds are formulated with suitable polymeric or hydrophobic materials (for example, as an emulsion in an acceptable oil) or ion exchange resins, or as sparingly soluble derivatives, for example, as a sparingly soluble salt.

In some embodiments, for buccal or sublingual administration, the compositions are in the form of tablets, lozenges, pastilles, or gels formulated in conventional manner. In some embodiments, such compositions comprise the active ingredient in a flavored basis such as sucrose and acacia or tragacanth.

In some embodiments, pharmaceutical compositions are formulated in rectal compositions such as suppositories or retention enemas, e.g., containing conventional suppository bases such as cocoa butter, polyethylene glycol, or other glycerides.

In some embodiments, pharmaceutical compositions are administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Pharmaceutical compositions suitable for topical administration include liquid or semi-liquid preparations suitable for penetration through the skin to the site of inflammation such as gels, liniments, lotions, creams, ointments or pastes, and drops suitable for administration to the eye, ear or nose. In some embodiments, the active ingredient comprises, for topical administration, from 0.001% to 10% w/w, for instance from 1% to 2% by weight of the formulation.

Pharmaceutical compositions for administration by inhalation are conveniently delivered from an insufflator, nebulizer pressurized packs or other convenient means of delivering an aerosol spray. In some embodiments, pressurized packs comprise a suitable propellant such as dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Alternatively, in some embodiments, for administration by inhalation or insufflation, pharmaceutical preparations take the form of a dry powder composition, for example a powder mix of the compound and a suitable powder base such as lactose or starch. In some embodiments, the powder composition is presented in unit dosage form, in for example, capsules, cartridges, gelatin or blister packs from which the powder is administered with the aid of an inhalator or insufflator.

It should be understood that in addition to the ingredients particularly mentioned above, the compounds and compositions described herein, in some embodiments, include other agents conventional in the art having regard to the type of formulation in question. For example, in some embodiments, the compounds and compositions described herein suitable for oral administration include flavoring agents.

### Methods of Dosing and Treatment Regimens

In one embodiment, the FXR agonists, anti-fibrotic therapeutic agents, anti-inflammatory agents or metabolic therapeutic agents described herein, or pharmaceutically acceptable salts thereof, are used in the preparation of medicaments for the treatment of diseases or conditions in a mammal that would benefit from administration of a FXR agonist. Methods for treating any of the diseases or conditions described herein in a mammal in need of such treatment, involve administration of pharmaceutical compositions that include the combination of at least one FXR agonist and an anti-fibrotic therapeutic agent or the combination of at least one FXR agonist and an anti-inflammatory agent or the combination of at least one FXR agonist and a metabolic therapeutic agent as described herein or pharmaceutically acceptable salts, active metabolites, prodrugs, or pharmaceutically acceptable solvates thereof.

Disclosed herein, are methods of administering a FXR agonist in combination with an additional therapeutic agent. In some embodiments, the additional therapeutic agent comprises a therapeutic agent for treatment of diabetes or diabetes related disorder or conditions, alcoholic or non-alcoholic liver disease, inflammation related intestinal conditions, or cell proliferative disorders.

In certain embodiments, the compositions containing the combination therapies described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

### Treatment Based on Biomarker Detection

In some embodiments, the administration of pharmaceutical compositions that include at least one FXR agonist is based on the patient's circulating or tissue-based FGF-19 levels. In some embodiments, the administration of pharmaceutical compositions that include the combination of at least one FXR agonist and an additional therapeutic agent is based on the patient's serum C4 (7α-hydroxy-4-cholesten-3-one) levels. In some embodiments, the administration of pharmaceutical compositions that include the combination of at least one FXR agonist and an additional therapeutic agent is based on the patient's serum bile acid levels. In some embodiments, the administration of pharmaceutical compositions that include the combination of at least one FXR agonist and an additional therapeutic agent is based on the patient's stool bile acid levels. In some embodiments, the additional therapeutic agent is an anti-fibrotic therapeutic agent, anti-inflammatory agent, a metabolic therapeutic agent, an anti-inflammatory agent, or any of the other therapeutic agents described herein. In some embodiments, the compositions containing the combination therapies described herein are administered to patients with abnormal FGF-19, C4 (7α-hydroxy-4-cholesten-3-one), or bile acid levels. In some embodiments, the compositions containing the combination therapies described herein are administered to patients with abnormal FGF-19, C4 (7α-hydroxy-4-cholesten-3-one), or bile acid levels for the treatment of any of the diseases or conditions described herein.

In prophylactic applications, compositions containing the combination therapies described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, which previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments, an FXR agonist and an additional therapeutic agent described herein are administered at a dose lower than the dose at which either the FXR agonist or the additional therapeutic agent are normally administered as monotherapy agents. In certain embodiments, an FXR agonist and an additional therapeutic agent described herein are administered at a dose lower than the dose at which either the FXR agonist or the additional therapeutic agent are normally administered to demonstrate efficacy. In certain embodiments, an FXR agonist is administered at a dose lower than the dose at which it is normally administered as a monotherapy agent, when administered in combination with an additional therapeutic agent described herein. In certain embodiments, an FXR agonist is administered at a dose lower than the dose at which it is normally administered to demonstrate efficacy, when administered in combination with an additional therapeutic agent described herein. In certain embodiments, an additional therapeutic agent is administered at a dose lower than the dose at which it is normally administered as a monotherapy agent, when administered in combination with an FXR agonist. In certain embodiments, an additional therapeutic agent is administered at a dose lower than the dose at which it is normally administered to demonstrate efficacy, when administered in combination with an FXR agonist.

In certain embodiments, wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained.

In certain embodiments, however, the patient requires intermittent treatment on a long-term basis upon any recurrence of symptoms. The amount of a given agent that corresponds to such an amount varies depending upon factors such as the particular compound, disease condition and its severity, the identity (e.g., weight, sex) of the mammal in need of treatment, but nevertheless is determined according to the particular circumstances surrounding the case, including, e.g., the specific agent being administered, the route of administration, the condition being treated, and the mammal being treated.

In general, however, doses employed for adult human treatment are typically in the range of about 0.01 mg to about 5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four, or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual mammal, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In certain instances, it is appropriate to administer at least one compound described herein, or a pharmaceutically acceptable salt thereof, in combination with one or more other therapeutic agents.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, a compound described herein, or a pharmaceutically acceptable salt thereof, is co-administered with a second therapeutic agent, wherein the compound described herein, or a pharmaceutically acceptable salt thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is, in some embodiments, additive of the two therapeutic agents or the patient experiences a synergistic benefit.

In certain embodiments, different dosages of the compounds disclosed herein will be utilized in formulating pharmaceutical composition and/or in treatment regimens when the compounds disclosed herein are administered in combination with one or more additional agent, such as an additional drug, an adjuvant or the like. Dosages of drugs and other agents for use in combination treatment regimens are optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt thereof, is initiated prior to, during, or after treatment with a second agent described herein, and continues until any time during treatment with the second agent or after termination of treatment with the second agent. It also includes treatments in which a compound described herein, or a pharmaceutically acceptable salt thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g. the disease, disorder or condition from which the mammal suffers; the age, weight, sex, diet, and medical condition of the mammal). Thus, in some instances, the dosage regimen actually employed varies and, in some embodiments, deviates from the dosage regimens set forth herein.

For combination therapies described herein, dosages of the co-administered compounds vary depending on the type of co-therapeutic agent employed, on the specific therapeutic agent employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to mammals with a propensity to develop conditions or diseases in order to prevent the occurrence of the disease or condition. In another embodiment, the compounds and compositions are administered to a mammal during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each mammal. For example, in specific embodiments, a compound described herein or a formulation containing the compound is administered for at least 2 weeks, about 1 month to about 5 years.

### EXAMPLES

The following examples are provided for illustrative purposes only and not to limit the scope of the claims provided herein.

### Example 1

### NASH Activity Study (STZ Model)

NASH is induced in male C57BL/6 by a single subcutaneous injection of 200 ug STZ 2 days after birth followed by feeding high fat diet (HFD) ad libitum after 4 weeks of age. While continuing HFD, a combination of an FXR agonist disclosed herein is dosed for 4-8 weeks to determine the effects on NASH. Fasting glucose is measured throughout the study with a hand held glucose meter. Serum alanine aminotransferase (ALT), aspartate aminotransferase (AST) and triglyceride (TG) are measured by a clinical chemistry analyzer. The contents of TG in the liver tissue are measured using the Triglyceride E-test kit (Wako, Tokyo, Japan). Histological analysis of liver sections is performed on tissue embedded in Tissue-TEK Optimal Cutting Temperature (O.C.T.) compound, snap frozen in liquid nitrogen, and stored at −80° C. The sections are cut (5 μm), air dried, and fixed in acetone. For hematoxylin and eosin (H&E) staining, liver sections are prefixed by Bouin's solution and then stained with hematoxylin and eosin solution. The degree of (zone-3) liver fibrosis is assessed with Sirius red staining.

### Example 2

### NASH Activity Study (AMLN model)

NASH is induced in male C57BL/6 mice by diet-induction with AMLN diet (DIO-NASH) (D09100301, Research Diet, USA) (40% fat (18% trans-fat), 40% carbohydrates (20% fructose) and 2% cholesterol). The animals are kept on the diet for 29 weeks. After 26 weeks of diet induction, liver biopsies are performed for base line histological assessment of disease progression (hepatosteatosis and fibrosis), stratified and randomized into treatment groups according to liver fibrosis stage, steatosis score, and body weight. Three weeks after biopsy the mice are stratified into treatment groups and dosed daily by oral gavage with a combination of an FXR agonist disclosed herein for 8 weeks. At the end of the study, liver biopsies are performed to assess hepatic steatosis and fibrosis by examining tissue sections stained with H&E and Sirius Red, respectively. Total collagen content in the liver is measured by colorimetric determination of hydroxyproline residues by acid hydrolysis of collagen. Triglycerides and total cholesterol content in liver homogenates are measured in single determinations using auto-analyzer Cobas C-111 with commercial kit (Roche Diagnostics, Germany) according to manufacturer's instructions.

### Example 3

### Intrahepatic Cholestasis Model

Experimental intrahepatic cholestasis induced by 17a-ethynylestradiol (EE2) treatment in rodents is a widely used in vivo model to examine the mechanisms involved in estrogen-induced cholestasis. Intrahepatic cholestasis is induced in adult male mice by subcutaneous injection of 10 mg/kg 17a-ethynylestradiol (EE2) daily for 5 days. Testing of a combination of an FXR agonist disclosed herein are performed by administration during EE2 induction of cholestasis. Cholestatic effects are quantitated by assessing liver/body weight ratio and measuring serum total bile acids and alkaline phosphatase levels are measured using reagents and controls from Diagnostic Chemicals Ltd. and the Cobas Mira plus CC analyzer (Roche Diagnostics). For histology and mitosis measurements, liver samples from each mouse are fixed in 10% neutral buffered formalin. Slides are stained with hematoxylin and eosin using standard protocols and examined microscopically for structural changes. Hepatocyte proliferation is evaluated by immunohistochemical staining for Ki67.

### Example 4

### Rat ANIT Model

A combination of an FXR agonist and an additional therapeutic agent described herein is evaluated in a chronic treatment model of cholestasis over a range of doses from 0.01 to 10 mg/kg. This model is used to evaluate the combination therapies described herein, for the treatment of cholestatic liver disorders such as bile acid malabsorption (e.g., primary or secondary bile acid diarrhea), bile reflux gastritis, collagenous colitis, lymphocytic colitis, diversion colitis, indeterminate colitis, Alagille syndrome, biliary atresia, ductopenic liver transplant rejection, bone marrow or stem cell transplant associated graft versus host disease, cystic fibrosis liver disease, and parenteral nutrition-associated liver disease.

Rats are treated with alpha-naphthylisothiocyanate (ANIT) (0.1% w/w) in food for 3 days prior to treatment with a compound described herein, at doses from 0.01 to 10 mg/kg (“Veh”). A noncholestatic control group is fed standard chow diet without ANIT, and serves as the noncholestatic control animals (“Control”). After 14 days of oral dosing, rat serum is analyzed for levels of analytes. LLQ, lower limit of quantitation. Mean±SEM; n=5. Levels of hepatobiliary injury indicators are measured in rat serum, such as elevated levels of circulating aspartate aminotransferase (AST), alanine aminotransferase (ALT), bilirubin and bile acids. ANIT exposure induces profound cholestasis and hepatocellular damage. A combination of an FXR agonist and an additional therapeutic agent described herein that improves many of these indicators is useful in the treatment of the aforementioned diseases or conditions.

### Example 5

### Mouse Chronic DSS Colitis Model

A chronic Dextran Sodium Sulfate (DSS)-induced mouse model is used to test the therapeutic potential of combination therapies described herein against inflammatory bowel disease (IBD). Chronic colitis is induced by feeding mice 2% DSS in drinking water for 5 days and regular drinking water for 5 days, then this feeding cycle is repeated two more times with a total of three cycles. Colitis develops approximately after the first cycle of DSS feeding, which is monitored by loss of body weight, stool consistency, and rectal bleeding. Combinations of an FXR agonist and an additional therapeutic agent described herein are tested by administering to mice at the same time of starting 2% DSS water feeding. Alternatively, testing of combination therapies is performed post the first feeding cycle of 2% DSS water and regular water. During the period of administering the combination therapies described herein to mice, the therapeutic effects are monitored by observations on body weights, stool consistency, and rectal bleeding. After euthanasia, the disease development and effects of the combination therapies described herein are further quantified by measuring colon weight and length, colon histology by H&E staining for inflammation and structural changes in mucosa, and protein and RNA expression of genes related to the disease.

### Example 6

### Adoptive T-cell Transfer Colitis Mouse Model

Adoptive T-cell transfer colitis model is accepted as a relevant mouse model for human inflammatory bowel disease (IBD). To induce colitis in this model, the CD4 T-lymphocyte population is isolated from the spleens of donor mice. Subsequently, a subpopulation of CD4+CD45RB high T-cells is purified by cell sorting using flow cytometry. The purified CD4+CD45RB high T-cells are injected into the peritoneal cavity of the recipient severe combined immunodeficiency (SCID) mice. Colitis develops approximately three to six weeks after T-cell transfer, which is monitored by loss of body weight, inconsistent stool or bloody diarrhea. Testing of combinations of an FXR agonist and an additional therapeutic agent described herein is initiated at the same time of injecting purified CD4+CD45RBhigh T-cells to the recipient SCID mice. Alternatively, the combinations of an FXR agonist and an additional therapeutic agent described herein are administered two or three weeks post T-cell transfer, when colitis has already developed in the model. During the period of administering the combinations of an FXR agonist and an additional therapeutic agent described herein to mice, the therapeutic effects may be monitored by observations on body weights, stool consistency, or rectal bleeding. After euthanasia, the disease development and effects of the compounds are further quantified by measuring colon weight and length, and colon histology by H&E staining for inflammation and structural changes in mucosa, and protein and RNA expression of genes related to the disease.

Results: CD4+CD45RB^{hi} T-cell transfer led to a 12% (p<0.05) reduction in body weight from baseline, which was reversed by (1R,4R)-4-hydroxy-N-(((1R,4R)-4-(4-methoxy-3-methylphenyl)cyclohexyl)methyl)-N-(3-(2-methoxythiazol-5-yl)phenyl)cyclohexane-1-carboxamide (Compound 2) (10 mg/kg), anti-IL-12p40 and CsA. A marker of colitis, colon W/L, was increased 2.8 fold (p<0.05) in the vehicle group, relative to control mice without T-cell transfer. As compared to vehicle, Compound 2 treated mice showed 41% and 38% reduction in colon W/L at 10 mg/kg and 30 mg/kg respectively (p<0.01). Treatment with anti-IL-12/23 and CsA showed 52% and 34% improvement in colon W/L, respectively. The vehicle treated mice averaged histopathology scores of 4, 3 and 2 for inflammation, hyperplasia and gland loss, respectively, with little or no erosion, and an average histopathology sum score of 10. Compound 2 at 10 and 30 mg/kg significantly reduced the sum score by 71% (p<0.01) and 74% (p<0.01), respectively, comparable to 78% reduction by anti-IL-12/23 (p<0.01). Both Compound 2 and anti-IL-12/23 showed similar improvement across all histopathology endpoints. While CsA improved the colon W/L it failed to show significant improvement in histopathology parameters.

Compound 2, a non-bile acid FXR agonist, is efficacious in reducing colitis in the adoptive T-cell transfer model with efficacy superior to CsA and comparable to anti-IL-12/23 treatment. Compound 2 represents a novel class of oral agents that may offer an alternative treatment for IBD.

### Example 7

### CCl₄ Fibrosis Model

Fibrosis is induced in BALB/c male mice by bi-weekly administration of CCl₄ administered by intraperitoneal injection. CCl₄ is formulated 1:1 in oil and is injected IP at 1 ml/kg. After 2-4 weeks of fibrosis induction, the combinations of an FXR agonist and an additional therapeutic agent described herein are administered daily by oral gavage for 2-6 weeks of treatment while continuing CCl₄ administration. At study termination, livers are formalin fixed and stained with Sirius Red stain for histopathological evaluation of fibrosis. Total collagen content is measured by colorimetric determination of hydroxyproline residues by acid hydrolysis of collagen. Serum alanine aminotransferase (ALT) and aspartate aminotransferase (AST) are measured by a clinical chemistry analyzer.

### Example 8

### PK/PD and Safety Assessment of Compound 1 in Healthy Subjects

Purpose: The purposes of this study was to assess the safety and tolerability of single and multiple oral doses of Compound 1, to characterize the pharmacokinetics (PK) of single and multiple oral doses of Compound 1, to characterize the pharmacodynamics (PD) of single and multiple oral doses of Compound 1, and to identify the recommended multiple oral dose level(s) of Compound 1 for future studies in patients. The study was a first-in-human, 2-part, single-center Phase 1, randomized, double-blind, placebo-controlled study in healthy male subjects.

Inclusion Criteria: Healthy male subjects aged 18 to 65 years with a BMI from 18.0 to 30.0 kg/m² and a weight greater than 60kg.

Subjects: Part A—56 healthy male subjects. Part B—60 healthy male subjects.

Study Drug: Compound 1, administered as an oral tablet.

Placebo: Same oral tablet as study drug, but without Compound 1.

### Variables

Safety: Adverse events, clinical laboratory, vital signs, 12-lead electrocardiogram, physical examinations.

PK: Plasma Compound 1 concentrations, Plasma PK parameters.

PD: Plasma levels of 7-alpha-hydroxy-4-cholesten-3-one (C4), plasma levels of fibroblast growth factor 19 (FGF19), and levels of total bile acids (for Part B only).

### Study Design

Part A—Single Ascending Dose [SAD]

Seven groups of eight healthy male subjects were administered single doses of Compound 1 at various oral dose levels ranging from 20-400 mg (20, 30, 50, 100, 15, 200, and 400 mg doses). Two subjects from each group were given placebo, and the remaining six subjects from each group received Compound 1.
Part B—Multiple Ascending Dose [MAD]

6 groups of 10 healthy male subjects each to investigate the safety, tolerability, PK, and PD of multiple oral doses of Compound 1. Subjects in all groups received ascending multiple oral doses of Compound 1 or matching placebo once daily on Days 1 to 14. Dosing was conducted under fasted conditions on each dosing day. Patients in each group received multiple oral doses of 20, 40, 50, 80, 100, and 150 mg of Compound 1 once daily on days 1 to 14 of the study. Eight members of each group received Compound 1, and two members of each group received placebo.

### Results

### Pharmacokinetics

### MAD Part

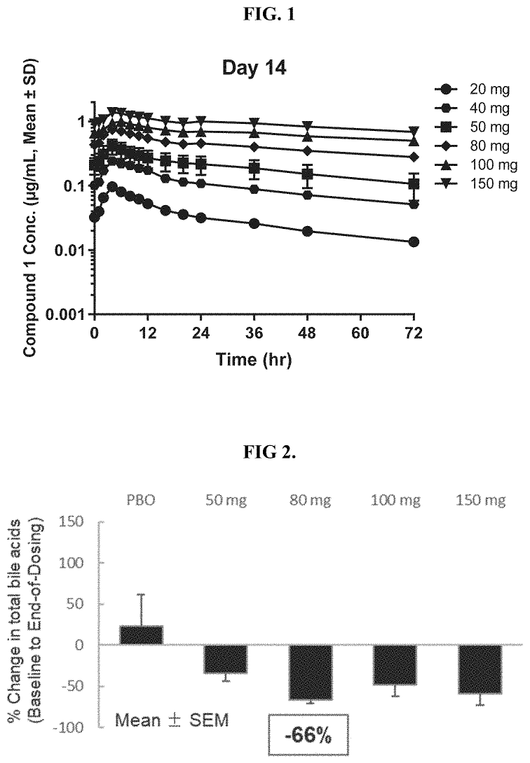

Following 14 days of oral dose administration of Compound 1 once daily in the range of 20 mg to 150 mg, an increase in plasma drug exposure with dose was observed as shown in FIG. 1. Compound 1 was determined to have high levels in the plasma at 24 hours following dosing, indicating a sustained PK profile.

### Pharmacodynamics

### MAD Part

### Bile Acid

FIG. 2 shows the resulting average change in bile acid levels for the placebo and 50 mg-150 mg/day cohorts at day 14. Each of the 50-150 mg cohorts receiving Compound 1 showed a decrease in bile acid levels.

### C4

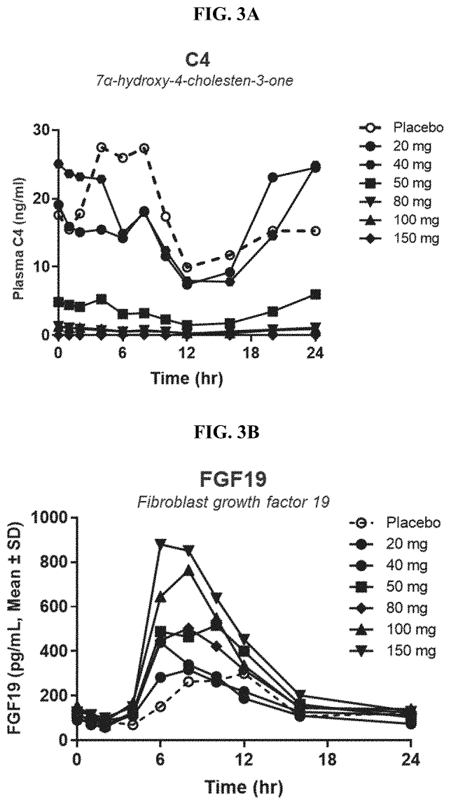

Arithmetic mean plots of absolute C4 plasma levels on Day 14 of the MAD part are presented in FIG. 3A. Following multiple doses of 20 mg to 150 mg of Compound 1 on Day 14, the mean C4 levels for most dose levels of Compound 1 were lower than for placebo treatment. The decrease in C4 levels was notable at 50 mg and above. The mean C4 levels remained decreased at 24 hours post dose on Day 14.

### FGF19

Following multiple doses of 20 mg to 150 mg of Compound 1, the mean FGF19 levels increased as shown in FIG. 3B. The maximum increase in mean FGF19 levels was attained at approximately 6 to 10 hours post dose.

### Example 9

### Clinical Trial for Non-Alcoholic Steatohepatitis (NASH)

A non-limiting example of a non-alcoholic steatohepatitis (NASH) clinical trial in humans is described below.

Purpose: The purposes of this study was to evaluate the safety and tolerability of Compound 1 monotherapy in subjects with NASH, to characterize the pharmacokinetics (PK) of Compound 1, to characterize the pharmacodynamics (PD) of Compound 1 dose and response, to estimate the activity of Compound 1 using Magnetic Resonance Imaging-Proton Density Fat Fraction (MRI-PDFF) and NASH fibrosis biomarkers.

Study Design: This is a two-part study in subjects with NASH, consisting of (Part 1) an open-label, uncontrolled, single-center assessment of 50 mg of Compound 1 for 4 weeks. Followed by (Part 2) a double-blind, placebo-controlled, multi-center assessment of 50-80 mg of study drug (Compound 1 or placebo) for 12 weeks.

Part 1: An open-label, uncontrolled, single-center study assessing 50 mg of Compound 1 treatment for 4 weeks; 10 subjects were enrolled.

Part 2: A double-blind, placebo-controlled, multi-center assessment of 50-80 mg of Compound 1 treatment for 12 weeks; approximately 55 subjects will be randomized to either Compound 1 or placebo in a 2:1 ratio. The first 31 subjects will be randomized to either 80 mg of Compound 1 or placebo. The remaining 24 subjects will be randomized to either 50 mg of Compound 1 or placebo. No dose adjustments will be allowed for individual subjects during the study.
Inclusion Criteria: (1) Male and female subjects 18 to 75 years old; (2) Diagnosis of NASH based on biopsy or imaging; (3) ≥10% liver fat content measured by MRI-PDFF during screening; (4) No investigational agent within 30 days (or 5 drug elimination half-lives) prior to first dose of study drug; (5) Subjects on GLP1 agonists, SGLT-2 inhibitors, or allowable statins must be on stable doses for at least three months prior to first dose of study drug; (6) Subjects may be on vitamin E at doses <800 IU/day, if the dose has been stable dose for at least 3 months prior to first dose of study drug.
Exclusion Criteria: (1) History or presence of any other liver disease (e.g., alcoholic liver disease, viral hepatitis, etc.) or history of liver transplant; (2) Presence of cirrhosis on any liver biopsy (stage 4 fibrosis); (3) Excessive consumption of alcohol; (4) Weight loss >10% in the 6 months prior to screening or >5% during screening; (5) Concomitant use of drugs that are strong or moderate CYP3A4 inhibitors, or CYP3A4 substrates with a narrow therapeutic index.
Efficacy Assessment: % liver fat content measured by MRI-PDFF and NASH biomarkers.
Safety Assessments: Safety assessments will include collection of adverse events, vital signs and physical examination, 12-lead ECG, laboratory assessments and verification of concomitant treatments. Laboratory tests and procedures may be done more frequently if clinically indicated.

Pharmacokinetic Assessments: Blood samples will be taken according to the PK sampling schedule. PK parameters (Cmax, tmax, t½, Ctrough, CLss/F, Cavg(0-24 h), AUC0-tau, AUC0-t, AUC0-inf) will be estimated by non-compartmental analysis.

Pharmacodynamic (PD) Assessments: Blood samples will be taken according to the PD (C4, FGF19, bile acid) sampling schedule.

Biomarker Assessments: Fibrosis measured by: Enhanced Liver Fibrosis (ELF) score derived from measuring hyaluronic acid, procollagen III amino terminal peptide (PIIINP), tissue inhibitor of metalloproteinase 1 (TIMP-1) as a biomarker for fibrosis; Pro-peptide of Type III Collagen (Pro-C3) as a biomarker for fibrosis; NAFLD Fibrosis Score (NFS) to identify advanced fibrosis (age, hyperglycemia, body mass index, platelet count, albumin, and AST/ALT ratio); FIB-4 Score to stage the level of fibrosis (age, AST, ALT, and the platelet count).

Bile Acid Composition: Serum bile acids (total and a panel of 15 bile acids measured by LC-MS); specific ratios and methods of analysis.

Primary Endpoints: Incidence, severity, and outcome of adverse events (AEs), serious adverse events (SAEs), and laboratory abnormalities.

Exploratory Endpoints: Activity estimated by liver fat quantification using MRI-PDFF.

Length of Study: Subjects enrolled in Part 1 participated in the study for approximately 12 weeks, which included a 4-week screening period, 4 weeks (28 days) for the treatment period, and 4 weeks for follow-up. Subjects enrolled in Part 2 will participate in the study for approximately 20 weeks, which includes an approximate 4-week screening period, 12 weeks (84 days) for the treatment period, and 4 weeks for follow-up.
Study Treatment (Part 1): Subjects enrolled in Part 1 received Compound 1 50 mg for 28 consecutive days.
Study Treatment (Part 2): Subjects enrolled in Part 2 will receive Compound 1 (80 mg or 50 mg) or matching placebo for 84 consecutive days.

### Results—Study Part 1

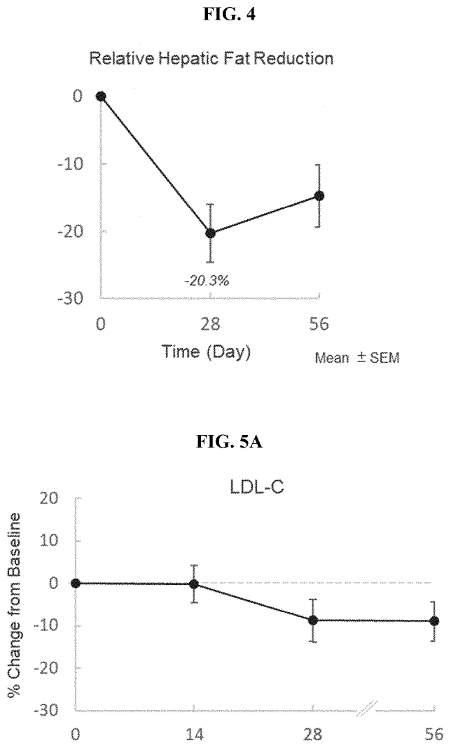

The 10 patients with NASH who enrolled in the study received 50 mg doses of Compound 1 daily for a period of 28 days as described above. Hepatic fat levels in each patient were assessed prior to treatment with Compound 1, after 28 days of treatment with Compound 1, then again 28 days after cessation of treatment with Compound 1. The resulting average changes in liver fat from baseline for the group is shown in FIG. 4, and indicates that patients displayed an average of 20.3% reduction in fatty liver deposits from baseline after 28 days of treatment with Compound 1.

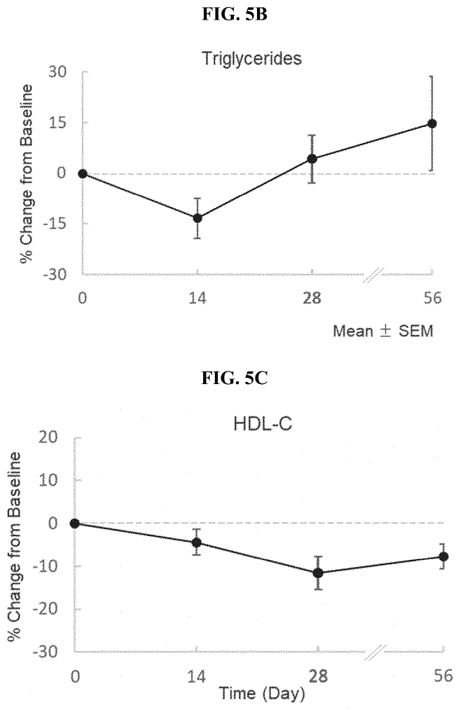

Serum lipid levels of the subjects were measured at start of treatment, 14 days after initiation of treatment, at the 28 day endpoint, and again 28 days after cessation of treatment. The resulting average changes from baseline for LDL-C, triglycerides, and HDL-C are shown in FIG. 5A, FIG. 5B, and FIG. 5C respectively.

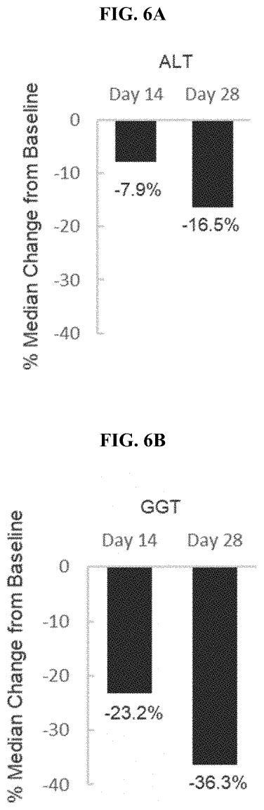

Levels of alanine aminotransferase (ALT) and gamma-glutamyl transferase (GGT) were assessed in subjects prior to administration of Compound 1, after 14 days of dosing of Compound 1, and after 28 days of dosing of Compound 1. The resulting percent changes from baseline for these biomarkers can be seen in FIG. 6A, FIG. 6B. ALT showed a decrease from baseline of 7.9% at day 14, and a further drop to 16.5% below baseline at day 28. GGT levels decreased 23.2% at day 13, and a 36.3% decrease from baseline by day 28.

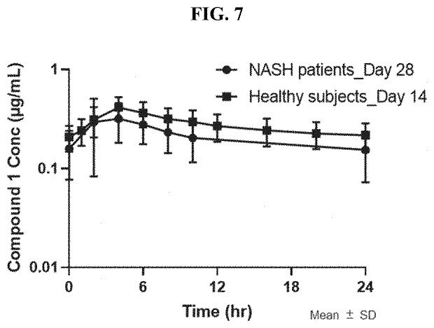

FIG. 7 shows plasma levels of Compound 1 during the treatment of both healthy patients from Example 8 and patients from Study Part 1 of Example 9. Blood plasma levels of Compound 1 were similar for both NASH and healthy patients throughout 24 hours after dosing with Compound 1. Compound 1 levels remained nearly constant throughout this 24 hour period both in healthy and NASH patients, suggesting Compound 1 has a sustained PK profile.

### Example 10

### Clinical Trial for Irritable Bowel Syndrome

A non-limiting example of an irritable bowel syndrome clinical trial in humans is described below.

Purpose: The purposes of this study are to characterize the safety, pharmacodynamics, and activity of Compound 1, or a pharmaceutically acceptable salt thereof, in subjects with diarrhea-predominant irritable bowel syndrome (IBS-D) with bile acid malabsorption (BAM).

Primary Objective: To evaluate the effect of Compound 1, or a pharmaceutically acceptable salt thereof, and placebo on a composite endpoint for the number and form of stools using the Bristol Stool Form Scale (BSFS).

Secondary Objectives: To characterize the safety and tolerability, characterize the effect on total fecal BA and the proportion of primary BA (% chenodeoxycholic acid [CDCA], % cholic acid [CA]) in stool, characterize the effect on fecal fat content, characterize the effect on each component of bowel function scoring (e.g., number of bowel movements, consistency, ease of passage, sense of completeness of evacuation), characterize the effect on IBS Global Symptom Score, characterize the effect on worst abdominal pain (WAP), characterize the effect on the proportion of patients that receive a rescue medication, characterize the effect on fasting serum C4 and FGF-19 levels, characterize the effect on colonic transit time (geometric center at 24 and 48 hours) at selected study sites, of Compound 1, or a pharmaceutically acceptable salt thereof, and placebo.

Inclusion Criteria: Male and female subjects 18 to 75 years old that meet Rome III criteria for IBS-D. Evidence of BAM as determined by one or more of the following criteria: Currently on bile acid sequestrant therapy with improvement in symptoms; Increased total fecal BA (fecal BA must be >2337 μmoles/48 hr) based on measurement within the last 60 days; Fasting serum C4 level of at least 52 ng/mL during Screening; Female subjects of child bearing potential must have a negative serum pregnancy test during Screening, agree to not become pregnant during the study, and agree to use birth control throughout the study and for up to 3 months after the last dose of Compound 1, or a pharmaceutically acceptable salt thereof. Male subjects of child-conceiving potential must agree to use birth control (double barrier method) during the study and for up to 1 month after the last dose of Compound 1, or a pharmaceutically acceptable salt thereof.

Exclusion Criteria: Presence of any other medical condition known to cause diarrhea or constipation (e.g., bowel surgery, ulcerative colitis, Crohn's disease, IBS with constipation, etc.); renal disease (e.g., serum creatinine of 2.5 mg/dL or greater); liver disease (e.g., aspartate transaminase >2.5 ULN and/or alanine transaminase >2.5 ULN); use of an investigational new drug within 30 days (or 5 drug elimination half-life's) prior to Screening; active, serious medical disease with likely life expectancy less than 2 years; Active substance abuse or alcoholism in the year prior to Screening; Pregnancy, planned pregnancy, potential for pregnancy (e.g., unwillingness to use effective birth control during the study), or breast feeding; any other medical condition or social circumstance, which in the opinion of the investigator would impede compliance with or hinder completion of the study.

Study Treatment: Each subject will receive a single oral daily dose of study drug (placebo or 10-300 mg Compound 1, or a pharmaceutically acceptable salt thereof) on Days 1 to 28; Placebo or Compound 1, or a pharmaceutically acceptable salt thereof, should be taken with at least 4 ounces of water in the morning, as close as possible to the same time each day. To minimize the potential effect of food on the absorption of Compound 1, or a pharmaceutically acceptable salt thereof, it should be taken 1 hour before or 2 hours after eating. If the morning dose of placebo or Compound 1, or a pharmaceutically acceptable salt thereof, is missed, it may be taken later in the same day (up to 12 hours from planned dose time); however, if a daily dose is missed entirely, it should not be made up on the next day (it should be documented as a missed dose).

Rescue Medication: Loperamide 2 mg twice daily as needed may be administered for uncontrolled diarrhea during the Treatment Period defined at least three stools with BSFS of 6 or greater in a day.

Efficacy Assessment: Composite endpoint for the number and form of stools: Number of stools x form of stools (BSFS 1-7)=Composite Score/Day; Compare Composite Score over a given week (7 days) from Screening through Treatment Period. Total BA and the proportion of primary BA (% CDCA, % CA) in stool (random spot stool collection): Compare the mean total BA in stool for screening to Week-4; Compare the mean CDCA and CA combined percentage in stool for screening to Week-4. Fecal fat content (random spot stool collection). Colonic transit time: Compare the mean geometric center at 24 and 48 hours for screening to Week-4 for sites capable of conducting this analysis. Bowel function: Compare the total scores for each component of the diary (number of bowel movements, consistency, ease of passage, and sense of completeness of evacuation) over a given week (7 days) from Screening through Treatment Period. IBS Global Symptom Score: Subjects asked, “How would you rate your IBS symptoms overall over the last 7 days?”; Compare the mean IBS Global Symptom Score (0=none, 1=mild, 2=moderate, 3=severe, and 4=very severe) for each week (7 days) from Screening through Treatment Period. Worst Abdominal Pain (WAP): The daily diary will include a WAP pain scale where 0=no pain and 10=worst pain imaginable; Compare the mean weekly WAP score for each week (7 days) from Screening through Treatment Period. Use of rescue medication: Compare the proportion of subjects receiving rescue medication during the treatment period.

Biomarkers: Fasting serum C4 and FGF-19 levels: exploratory analyses will be performed to evaluate the relationship between treatment and the level of each biomarker. In addition, the relationship between each biomarker and efficacy endpoints will be explored. Colonic transit, if available.

Primary Endpoints: Mean composite score over a week for number and form of stools using BSFS from Screening (7 days prior to randomization) to Week 4.

Secondary Endpoints: Mean composite score over a week for number and form of stools using BSFS from Screening (7 days prior to randomization) to Week 1, Week 2, and Week 3; Mean composite score for two (2) highest values in a week for number and form of stools using BSFS from Screening (7 days prior to randomization) to Week 1, Week 2, Week 3, and Week 4; Mean composite score over a week for number of stools from Screening (7 days prior to randomization) to Week 1, Week 2, Week 3, and Week 4; Mean composite score over a week for form of stools using BSFS from Screening (7 days prior to randomization) to Week 1, Week 2, Week 3, and Week 4; Mean weekly WAP score from Screening (7 days prior to randomization) to Week 1, Week 2, Week 3, and Week 4; Mean weekly IBS Global Symptom Score from Screening (7 days prior to randomization) to Week 1, Week 2, Week 3, and Week 4; Mean total fecal BA and primary BA (% chenodeoxycholic acid [CDCA], % cholic acid [CA]) in stool from Screening to Week 4; Mean total fecal fat content in stool from Screening to Week 4; Correlation between fasting serum C4 and FGF-19 levels to each assessment of efficacy; Mean total colonic transit time (geometric center at 24 and 48 hours) at Screening and Week 4—performed at selected study sites.

### Example 11

### Clinical Trial for Ulcerative Colitis

A non-limiting example of an ulcerative colitis clinical trial in humans is described below.

Purpose: The purposes of this study are to characterize the safety, pharmacodynamics, and activity of Compound 1, or a pharmaceutically acceptable salt thereof, in subjects with moderate to severe ulcerative colitis.

Primary Objectives: To evaluate the impact of Compound 1, or a pharmaceutically acceptable salt thereof, in UC by comparing mean changes in the UC-100 Score as compared to placebo at Week 12.

Secondary Objectives: To evaluate the change in 3-component Mayo Score (score range 0-9 based on stool frequency, rectal bleeding, and findings on endoscopy), evaluate the effect of Compound 1, or a pharmaceutically acceptable salt thereof, and placebo on the Ulcerative Colitis Endoscopic Index of Severity (UCEIS), evaluate the effect of Compound 1, or a pharmaceutically acceptable salt thereof, and placebo on the Robarts Histologic Index (RHI), evaluate the change in total Mayo Score, evaluate changes in components of the Mayo Score (stool frequency, rectal bleeding, endoscopic score), assess clinical response (decrease in the Mayo score from baseline of 30% or more and 3 or more points, along with either a rectal bleeding subscore of 0 or 1 or a decrease in the rectal bleeding subscore of 1 point or more), assess clinical remission (Mayo score of 2 or fewer points, along with not having more than point in any individual subscore), evaluate the change in histologic index, evaluate the need for rescue medications, evaluate the effect on the Inflammatory Bowel Disease Questionnaire (IBDQ), assess changes in fecal calprotectin levels and serum C-reactive protein levels, the effect on fasting serum C4 and FGF-19 levels, between Compound 1, or a pharmaceutically acceptable salt thereof, and placebo at Week 12.

Study Treatment: Each subject will receive a single oral daily dose of study drug (placebo or Compound 1, or a pharmaceutically acceptable salt thereof) on Days 1 to 84. Allowed Concomitant Medications: If the subject is on a stable dose of corticosteroid (maximum of prednisone 30 mg/day or entocort 6 mg/day) for at least 2 weeks prior to the screening endoscopy, the corticosteroid may be continued during the screening, treatment, and follow-up periods if there are no dose adjustments. If the subject is on a stable dose of oral aminosalicylates, azathioprine, 6-mercaptopurine, or methotrexate for at least 3 weeks prior to the screening endoscopy, the medication may be continued during the screening, treatment, and follow-up periods if there are no dose adjustments. Prohibited Medications: Subjects must stop use of anti-tumor necrosis factor (TNF) therapy, ustekinumab, or vedolizumab ≥8 weeks before first dosing. Subjects must stop any investigational medication, medication for UC (except the allowed concomitant medications), or medication that affects bowel function at least 8 weeks prior to the screening endoscopy (i.e., washout period). These medications also may not be administered during the screening, treatment, and follow-up periods to avoid confounding the analysis of the data.

Inclusion Criteria: Male and female subjects 18 to 75 years old with a diagnosis of UC for at least 3 months prior to screening. Moderate to severe active UC defined by Mayo Score of 6 to 12 inclusive (range of 0-12) and an endoscopy score of at least 2 (range 0-3) with at least 15 cm of involved tissue during Screening. Central reading of endoscopy score must be done. Subjects who have previously received anti-tumor necrosis factor (TNF) therapy, ustekinumab, or vedolizumab must have discontinued therapy ≥8 weeks before first dosing (i.e. baseline). Either currently receiving treatment with, or have a history of failure to respond to, or tolerate, at least 1 of the following therapies: oral 5-aminosalicylate, oral corticosteroids, methotrexate, 6-mercaptopurine and azathioprine. Female subjects of child bearing potential must have a negative serum pregnancy test during screening, agree to not become pregnant during the study, and agree to use a form(s) of birth control throughout the study and for up to 3 months after the last dose of Compound 1, or a pharmaceutically acceptable salt thereof. Male subjects of child-conceiving potential must agree to use birth control (double barrier method) during the study and for up to 1 month after the last dose of Compound 1, or a pharmaceutically acceptable salt thereof.

Exclusion Criteria: Diagnosis of Crohn's disease or indeterminate colitis or the presence or history of a fistula consistent with Crohn's disease or microscopic colitis or radiation colitis or ischemic colitis; Presence of severe extensive colitis likely to require surgical intervention within 12 weeks of screening; Confirmed or suspected infection of the intestinal tract. Subject may be re-screened once the infection clears; Renal disease (e.g., serum creatinine of 2.5 mg/dL or greater); Liver disease (e.g., aspartate transaminase >2.5 ULN and/or alanine transaminase >2.5 ULN); Active, serious medical disease with likely life expectancy less than 2 years; Active substance abuse or alcoholism in the year prior to screening; Pregnancy, planned pregnancy, potential for pregnancy (e.g., unwillingness to use effective birth control during the study), or breast feeding.

Efficacy Assessment: UC-100 Score: Composite score based on endoscopy, histology, and stool frequency; Ulcerative Colitis Endoscopic Index of Severity (UCEIS): Endoscopic scoring of 3 domains: Vascular pattern (score 1-3), bleeding (score 1-4), and erosions and ulcers (score 1-4). The mean change in score from baseline to Week 12 will be compared between treatment groups. Robarts Histologic Index (RHI): Histologic scoring of 4 domains: chronic inflammatory infiltrate (score 0-3), lamina propria neutrophils (score 0-3), neutrophils in epithelium (score 0-3), and erosion or ulceration (score 0-3). The mean change in score from baseline to Week 12 will be compared between treatment groups. Mayo Score (MS): Total MS (score 1-12)—4 domains include stool frequency (score 0-3), rectal bleeding (score 0-3), endoscopy (score 0-3), and physicians global assessment (score 0-3); Partial MS (score 0-9)—does not include endoscopy score; Endoscopic MS (score 0-3)—endoscopic evaluation of the mucosa; The mean change in each score from baseline to Week 12 will be compared between treatment groups; Proportion of subjects with a clinical response: Defined as a reduction in total MS of ≥3 and ≥30% from baseline, with a decrease from baseline in the rectal bleeding subscore of ≥1 or an absolute rectal bleeding subscore of ≤1. The proportions will be compared between treatment groups at Week 12. Proportion of subjects with a clinical remission: Defined as MS of ≤2 and with no individual subscore >1. The proportions will be compared between treatment groups at Week 12. Proportion of subjects with an endoscopic response: Defined as MS endoscopy subscore ≤1. The proportions will be compared between treatment groups at Week 12. Proportion of subjects with a histologic remission at Week 12. Use of rescue medications: The proportion of subjects requiring each rescue medication during the treatment period will be compared. Short Inflammatory Bowel Disease Questionnaire (IBDQ) score: 10 question IBDQ. The mean change in score from baseline to Week 12 will be compared between treatment groups.

Biomarkers: Fasting serum C4 and FGF-19 levels; Exploratory analyses will be performed to evaluate the relationship between treatment and the level of each biomarker. In addition, the relationship between each biomarker and efficacy endpoints will be explored.

Primary Endpoint: Mean change in UC-100 at Week 12

Secondary Endpoints: Mean change in 3-component Mayo Score (score range 0-9 based on stool frequency, rectal bleeding, and findings on endoscopy) at Week 12; To evaluate the effect of Compound 1, or a pharmaceutically acceptable salt thereof, and placebo on the Ulcerative Colitis Endoscopic Index of Severity (UCEIS) at Week 12]; To evaluate the effect of Compound 1, or a pharmaceutically acceptable salt thereof, and placebo on the Robarts Histologic Index (RHI) at Week 12; Mean change in total Mayo Score at Week 12; Mean change in endoscopic Mayo Score at Week 12; Mean change in stool frequency and rectal bleeding subscores of Mayo Score at Week 12; Proportion of patients with clinical response as determined by total Mayo Score at Week 12; Proportion of patients with clinical remission as determined by total Mayo Score at Week 12; Mean change in histologic index at Week 12; Proportion of subjects with a histologic remission at Week 12; Proportion of subjects requiring each rescue medication during the treatment period; Mean change in Inflammatory Bowel Disease Questionnaire (IBDQ) score at Week 12; Mean change in fasting serum C4 and FGF-19 levels from baseline to Week 12; Mean change in fecal calprotectin levels from baseline to Week 12; Mean change in serum C-reactive protein levels from baseline to Week 12.

### Example 12

### In Vitro FXR Assay (TK)

### Seeding

CV-1 cells were seeded at a density of 2,000,000 cells in a T175 flask with DMEM+10% charcoal double-stripped FBS and incubated at 37° C. in 5% CO₂ for 18 h (O/N).

### Transfection

After 18 h of incubation, the medium in the T175 flask was changed with fresh DMEM+10% charcoal super-stripped serum. In a polypropylene tube, 2500 μL OptiMEM (Life Technologies, Cat #31985-062) was combined with expression plasmids for hFXR, hRXR, TK-ECRE-luc and pCMX-YFP. The tube was then briefly vortexed and incubated at room temperature for 5 minutes. Transfection reagent (X-tremeGENE HP from Roche, Cat #06 366 236 001) was added to the OptiMEM/plasmid mixture vortexed and incubated at room temperature for 20 minutes. Following incubation, the transfection reagent/DNA mixture complex was added to cells in the T175 flask and the cells were incubated at 37° C. in 5% CO₂ for 18 h (O/N).

### Test Compounds

Compounds were serially diluted in DMSO and added to transfected CV-1 cells. The cells were then incubated for 18 hrs. The next day cells were lysed and examined for luminescence.

Representative data for exemplary compounds disclosed herein is presented in the following table.

| Compound No | TK hFXR: EC₅₀ (uM) |
|---|---|
| Compound 1 | ≤0.25 uM |

### Example 13-A

### Parenteral Pharmaceutical Composition

To prepare a parenteral pharmaceutical composition suitable for administration by injection (subcutaneous, intravenous), 1-1000 mg of a compound described herein, or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Example 13-B

### Oral Solution

To prepare a pharmaceutical composition for oral delivery, a sufficient amount of a compound described herein, or a pharmaceutically acceptable salt thereof, is added to water (with optional solubilizer(s), optional buffer(s) and taste masking excipients) to provide a 20 mg/mL solution.

### Example 13-C

### Oral Tablet

A tablet is prepared by mixing 20-50% by weight of a compound described herein, or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example 13-D

### Oral Capsule

To prepare a pharmaceutical composition for oral delivery, 10-500 mg of a compound described herein, or a pharmaceutically acceptable salt thereof, is mixed with starch or other suitable powder blend. The mixture is incorporated into an oral dosage unit such as a hard gelatin capsule, which is suitable for oral administration.

In another embodiment, 10-500 mg of a compound described herein, or a pharmaceutically acceptable salt thereof, is placed into Size 4 capsule, or size 1 capsule (hypromellose or hard gelatin) and the capsule is closed.

### Example 14

### Efficacy Studies for Treatment of Cholangiocarcinoma and Hepatocellular Carcinoma (Patient Derived Xenograft Models)

Tumor tissue derived from patients with cholangiocarcinoma or hepatocellular carcinoma is engrafted into immunodeficient mice to develop tumors that retain histological/pathological architecture of the patient tumor and primary driver mutations and gene expression. The growth of these patient-derived xenografts (PDX) is monitored to examine the effect of test articles on tumor growth. Mice are inoculated subcutaneously, in the right flank, with a 2-3 mm diameter piece of freshly excised tumor from mice bearing the established primary human tumor tissue. The tumor is allowed to establish and when the mean tumor size reaches approximately 150 mm³, mice are randomized into treatment groups and treated daily oral dosing with vehicle control or the experimental compound. Tumor volumes are measured twice a week in two dimensions using an electronic caliper and volume is determined using the formula: V=(L×W×W)/2, where V is tumor volume, L is tumor length (the longest tumor dimension) and W is tumor width (the longest tumor dimension perpendicular to L). Mice are dosed for up to 4 weeks or until the tumor volume exceeds 3000 mm³ or the animal's body weight decreases greater than 20%.

The examples and embodiments described herein are for illustrative purposes only and various modifications or changes suggested to persons skilled in the art are to be included within the spirit and purview of this application and scope of the appended claims.

### Example 15

### Efficacy Studies for Treatment of Cholestasis and Primary Sclerosing Cholangitis (Mdr2^{-/-} Mouse Model)

Multidrug resistance 3 (MDR3) is responsible for transporting phospholipids into bile. Mutations on this transporter in humans can result in progressive familial intrahepatic cholestasis (PFIC3). Genetic knockout of the mouse homolog MDR2, similarly results in cholestasis and fibrosis in mice (Fickert 2004 Gastroenterology 127 261). This model can be used to assess the efficacy of FXR agonists to decrease cholestasis and liver injury (Baghdasaryan 2011 Hepatology 54 1313).

MDR2^{-/-} mice at 8 weeks of age exhibit elevated serum bile acids, liver enzymes and show evidence of hepatic fibrosis and inflammation. To examine therapeutic efficacy of FXR agonists, 8-week-old knockout mice can be dosed with compounds by oral gavage. Efficacy can be monitored by examining effects on serum bile acids, liver enzymes (ALT, ALP) and bilirubin. Additional efficacy points can include liver histopathology analysis and scoring of inflammation, bile duct hyperplasia and liver fibrosis.

What is claimed is:

1. A method of treating or preventing a liver disease or condition, a lipid disease or disorder, a metabolic inflammation-mediated disease or disorder, or a combination thereof comprising administering to a subject in need thereof a compound that has the following structure of Compound 1: 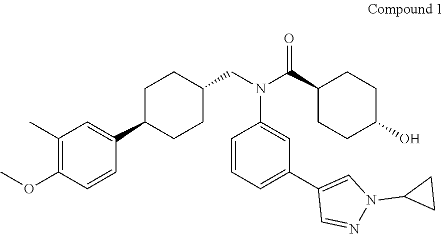 [SMILES: COc1ccc([C@H]2CC[C@H](CN(c3cccc(-c4cnn(C5CC5)c4)c3)C(=O)[C@H]3CC[C@H](O)CC3)CC2)cc1C]
or a pharmaceutically acceptable salt or solvate thereof.

2. The method of claim 1, wherein the liver disease or condition is steatohepatitis, cholangitis, fatty liver disease, cholestasis, cirrhosis, fibrotic liver disease, liver inflammation, primary biliary cholangitis, biliary atresia, Alagille syndrome, IFALD (intestinal failure associated liver disease), parental nutrition associated liver disease (PNALD), hepatitis, hepatocellular carcinoma, cholangiocarcinoma, or combinations thereof.

3. The method of claim 2, wherein the steatohepatitis is nonalcoholic steatohepatitis (NASH), alcoholic steatohepatitis (ASH), or HIV-associated steatohepatitis.

4. The method of claim 1, wherein the liver disease or condition is nonalcoholic steatohepatitis (NASH).

5. The method of claim 4, wherein the liver disease or condition is NASH that is accompanied by liver fibrosis.

6. The method of claim 4, wherein the liver disease or condition is NASH without liver fibrosis.

7. The method of claim 2, wherein the cholangitis is primary biliary cholangitis (PBC) or primary sclerosing cholangitis (PSC).

8. The method of claim 2, wherein the fatty liver disease is non-alcoholic fatty liver disease (NAFLD) or alcohol-related fatty liver disease.

9. The method of claim 2, wherein the cholestasis is intrahepatic cholestasis or extrahepatic cholestasis.

10. The method of claim 2, wherein the cholestasis is intrahepatic cholestasis of pregnancy or progressive familial intrahepatic cholestasis (PFIC).

11. The method of claim 2, wherein the cirrhosis is HIV-associated cirrhosis.

12. The method of claim 1, wherein the metabolic inflammation-mediated disease or disorder is diabetes mellitus.

13. The method of claim 12, wherein the diabetes mellitus is diabetes mellitus type 2.

14. The method of claim 1, wherein the lipid disease or disorder is dyslipidemia.

15. The method according to claim 2, wherein the fibrotic liver disease is a fibrotic liver disease resulting from nonalcoholic steatohepatitis (NASH), alcoholic steatohepatitis (ASH), non-alcoholic fatty liver disease (NAFLD), primary biliary cholangitis (PBC), primary sclerosing cholangitis (PSC), hepatitis C virus (HCV), cirrhosis, Wilson's disease, HIV associated steatohepatitis, HIV associated cirrhosis, or congenital hepatic fibrosis.

16. The method of claim 2, wherein the liver inflammation is acute hepatitis, chronic hepatitis, fulminant hepatitis, viral hepatitis, bacterial hepatitis, parasitic hepatitis, toxic- and drug-induced hepatitis, alcoholic hepatitis, autoimmune hepatitis, non-alcoholic steatohepatitis (NASH), neonatal hepatitis, or ischemic hepatitis.

17. The method according to claim 2, wherein the hepatitis is autoimmune hepatitis.

18. The method according to claim 2, wherein the liver disease or condition is Alagille syndrome.

19. The method according to claim 2, wherein the liver disease or condition is biliary atresia.

20. The method according to claim 2, wherein the liver disease or condition is hepatocellular carcinoma.

21. The method according to claim 2, wherein the liver disease or condition is cholangiocarcinoma.

22. The method of any one of claims 1-21, wherein Compound 1, or a pharmaceutically acceptable salt or solvate thereof, is systemically administered to the subject.

23. The method of any one of claims 1-21, wherein Compound 1, or a pharmaceutically acceptable salt or solvate thereof, is administered to the subject orally, by injection or intraveneously.

24. The method of any one of claims 1-23, further comprising administering to the subject an additional therapeutic agent in addition to Compound 1, or a pharmaceutically acceptable salt or solvate thereof.

25. A method of treating or preventing a gastrointestinal disease or condition, comprising administering to a subject in need thereof a compound that has the following structure of Compound 1: 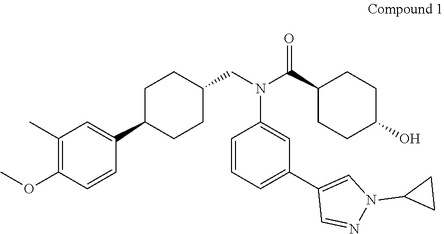 [SMILES: COc1ccc([C@H]2CC[C@H](CN(c3cccc(-c4cnn(C5CC5)c4)c3)C(=O)[C@H]3CC[C@H](O)CC3)CC2)cc1C]
or a pharmaceutically acceptable salt or solvate thereof.

26. The method of claim 25, wherein the gastrointestinal disease or condition is necrotizing enterocolitis, inflammatory bowel disease (IBD), irritable bowel syndrome (IBS), gastroenteritis, radiation induced enteritis, pseudomembranous colitis, enteritis, celiac disease, post-surgical inflammation of the intestines, graft versus host disease, bile acid reflux or colorectal cancer.

27. The method of claim 25, wherein the gastrointestinal disease or condition is inflammatory bowel disease (IBD).

28. The method of claim 27, wherein the inflammatory bowel disease (IBD) is Crohn's disease or ulcerative colitis.

29. The method of claim 26, wherein the irritable bowel syndrome (IBS) is irritable bowel syndrome with diarrhea (IBS-D), irritable bowel syndrome with constipation (IBS-C), mixed IBS (IBS-M), unsubtyped IBS (IBS-U), or bile acid diarrhea (BAD).

30. The method of claim 29, wherein the IBS-D is due to bile acid malabsorption.

31. The method of claim 25, wherein the gastrointestinal disease or condition is colitis.

32. The method of claim 31, wherein the colitis is ulcerative colitis, microscopic colitis, or pseudomembranous colitis.

33. The method of claim 26, wherein the enteritis is radiation-induced enteritis or chemotherapy-induced enteritis.

34. The method of claim 26, wherein the gastroenteritis is idiopathic gastroenteritis.

35. The method of claim 25, wherein the gastrointestinal disease or condition is bile acid reflux that is accompanied by gastro-esophageal reflux disease (GERD).

36. The method of claim 25, wherein the gastrointestinal disease or condition is bile acid reflux without GERD.

37. The method of any one of claims 25-36, wherein Compound 1, or a pharmaceutically acceptable salt or solvate thereof, is systemically administered to the subject.

38. The method of any one of claims 25-36, wherein Compound 1, or a pharmaceutically acceptable salt or solvate thereof, is non-systemically administered to the subject.

39. The method of any one of claims 25-38, wherein Compound 1, or a pharmaceutically acceptable salt or solvate thereof, is administered to the subject orally, by injection or intraveneously.

40. The method of any one of claims 25-38, further comprising administering to the subject an additional therapeutic agent in addition to Compound 1, or a pharmaceutically acceptable salt or solvate thereof.

41. A method of treating or preventing a renal disease or condition, comprising administering to a subject in need thereof a compound that has the following structure of Compound 1: 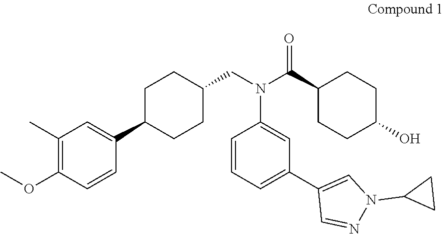 [SMILES: COc1ccc([C@H]2CC[C@H](CN(c3cccc(-c4cnn(C5CC5)c4)c3)C(=O)[C@H]3CC[C@H](O)CC3)CC2)cc1C]
or a pharmaceutically acceptable salt or solvate thereof

42. The method of claim 41, wherein the renal disease or condition is kidney fibrosis, acute kidney injury, chronic kidney injury, ischemic nephropathy, diabetic nephropathy, tubulointerstitial nephritis/nephropathy, glomerulonephritis/nephropathy, or combinations thereof.

43. The method of claim 41 or claim 42, wherein Compound 1, or a pharmaceutically acceptable salt or solvate thereof, is systemically administered to the subject.

44. The method of any one of claims 41-43, wherein Compound 1, or a pharmaceutically acceptable salt or solvate thereof, is administered to the subject orally, by injection or intraveneously.

45. The method of any one of claims 41-44, further comprising administering to the subject an additional therapeutic agent in addition to Compound 1, or a pharmaceutically acceptable salt or solvate thereof.

46. A method of treating or preventing cancer, comprising administering to a subject in need thereof a compound that has the following structure of Compound 1: 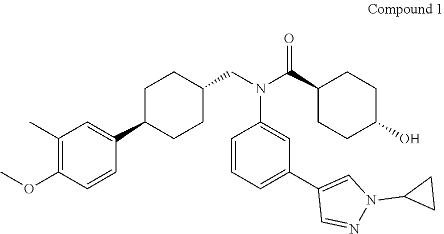 [SMILES: COc1ccc([C@H]2CC[C@H](CN(c3cccc(-c4cnn(C5CC5)c4)c3)C(=O)[C@H]3CC[C@H](O)CC3)CC2)cc1C]
or a pharmaceutically acceptable salt or solvate thereof.

47. The method of claim 46, wherein the cancer is prostate cancer, colorectal cancer, or hepatocellular carcinoma.

48. The method of claim 46 or claim 47, wherein Compound 1, or a pharmaceutically acceptable salt or solvate thereof, is systemically administered to the subject.

49. The method of any one of claims 46-48, wherein Compound 1, or a pharmaceutically acceptable salt or solvate thereof, is administered to the subject orally, by injection or intraveneously.

50. The method of any one of claims 46-49, further comprising administering to the subject an additional therapeutic agent in addition to Compound 1, or a pharmaceutically acceptable salt or solvate thereof.
